SEQUENCE LISTING (463 sequences)

SEQ ID NO: 1 - protein, 31 aa, Artificial Sequence
  source: /note=“Description of Artificial Sequence Synthetic peptide hybrid of human peptides”
YSEGTFTSDYSKLLEEEEAVRLFIEWLLAGG

SEQ ID NO: 2 - protein, 29 aa, Artificial Sequence
  source: /note=“Description of Artificial Sequence Synthetic peptide hybrid of human peptides”
YSEGTFTSDYSKLLEEEAVRLFIEWLLAG

SEQ ID NO: 3 - protein, 29 aa, Artificial Sequence
  source: /note=“Description of Artificial Sequence Synthetic peptide hybrid of human peptides”
YSEGTFTSDVSKLLEEEAVRLFIEWLLAG

SEQ ID NO: 4 - protein, 29 aa, Artificial Sequence
  source: /note=“Description of Artificial Sequence Synthetic peptide hybrid of human peptides”
YSEGTFTSDYSKLLDEEAVRLFIEWLLAG

SEQ ID NO: 5 - protein, 29 aa, Artificial Sequence
  source: /note=“Description of Artificial Sequence Synthetic peptide hybrid of human peptides”
YSEGTFTSDYSKLLEEEAVRLFIEWLVKG

SEQ ID NO: 6 - protein, 30 aa, Artificial Sequence
  source: /note=“Description of Artificial Sequence Synthetic peptide hybrid of human peptides”
YSEGTFTSDYSKLLEEEAVRLFIEWLLAGN

SEQ ID NO: 7 - protein, 35 aa, Artificial Sequence
  source: /note=“Description of Artificial Sequence Synthetic peptide hybrid of human peptides”
YSEGTFTSDYSKLLEEEAVRLFIEWLLAGGHNITQ

SEQ ID NO: 8 - protein, 30 aa, Artificial Sequence
  source: /note=“Description of Artificial Sequence Synthetic peptide hybrid of human peptides”
YSEGTFTSDYSKLLEEEAVRLFIEWLLAGG

SEQ ID NO: 9 - protein, 30 aa, Artificial Sequence
  source: /note=“Description of Artificial Sequence Synthetic peptide hybrid of human peptides”
YSEGTFTSDYSKLLEEEAVRLFIEWLLAGP

SEQ ID NO: 10 - protein, 38 aa, Artificial Sequence
  source: /note=“Description of Artificial Sequence Synthetic peptide hybrid of human peptides”
YSEGTFTSDYSKLLEEEAVRLFIEWLLAGPSSGAPPPS

SEQ ID NO: 11 - protein, 30 aa, Artificial Sequence
  source: /note=“Description of Artificial Sequence Synthetic peptide hybrid of human peptides”
YSEGTFTSDYSKLLEEEAVRLFIEWLLAQK

SEQ ID NO: 12 - protein, 30 aa, Artificial Sequence
  source: /note=“Description of Artificial Sequence Synthetic peptide hybrid of human peptides”
YSEGTFTSDYSKLLEEEAVRLFIEWLKNGG

SEQ ID NO: 13 - protein, 39 aa, Artificial Sequence
  source: /note=“Description of Artificial Sequence Synthetic peptide hybrid of human peptides”
YSEGTFTSDYSKLLEEEAVRLFIEWLKNGGPSSGAPPPS

SEQ ID NO: 14 - protein, 30 aa, Artificial Sequence
  source: /note=“Description of Artificial Sequence Synthetic peptide hybrid of human peptides”
YSEGTFTSDYSKLLEEEAVRLFIEWLKNGT

SEQ ID NO: 15 - protein, 30 aa, Artificial Sequence
  source: /note=“Description of Artificial Sequence Synthetic peptide hybrid of human peptides”
YSEGTFTSDYSKLLEEEAVRLFIEWLVKGP

SEQ ID NO: 16 - protein, 30 aa, Artificial Sequence
  source: /note=“Description of Artificial Sequence Synthetic peptide hybrid of human peptides”
YSQGTFTSDYSKLLEEEAVRLFIEWLVKGP

SEQ ID NO: 17 - protein, 30 aa, Artificial Sequence
  source: /note=“Description of Artificial Sequence Synthetic peptide hybrid of human peptides”
YSEGTFTSDLSKLLEEEAVRLFIEWLVKGP

SEQ ID NO: 18 - protein, 30 aa, Artificial Sequence
  source: /note=“Description of Artificial Sequence Synthetic peptide hybrid of human peptides”
YSEGTFTSDYSSYMEEEAVRLFIEWLVKGP

SEQ ID NO: 19 - protein, 30 aa, Artificial Sequence
  source: /note=“Description of Artificial Sequence Synthetic peptide hybrid of human peptides”
YSEGTFTSDYSIAMDEEAVRLFIEWLVKGP

SEQ ID NO: 20 - protein, 30 aa, Artificial Sequence
  source: /note=“Description of Artificial Sequence Synthetic peptide hybrid of human peptides”
YSEGTFTSDYSKLLEGQAAKLFIEWLVKGP

SEQ ID NO: 21 - protein, 30 aa, Artificial Sequence
  source: /note=“Description of Artificial Sequence Synthetic peptide hybrid of human peptides”
YSEGTFTSDYSKLLEKIHQQLFIEWLVKGP

SEQ ID NO: 22 - protein, 30 aa, Artificial Sequence
  source: /note=“Description of Artificial Sequence Synthetic peptide hybrid of human peptides”
YSEGTFTSDYSKLLEEEAQRLFIEWLVKGP

SEQ ID NO: 23 - protein, 30 aa, Artificial Sequence
  source: /note=“Description of Artificial Sequence Synthetic peptide hybrid of human peptides”
YSEGTFTSDYSKLLEEEAVREFIAWLVKGP

SEQ ID NO: 24 - protein, 30 aa, Artificial Sequence
  source: /note=“Description of Artificial Sequence Synthetic peptide hybrid of human peptides”
YSEGTFTSDYSKLLEEEAVRDFVNWLVKGP

SEQ ID NO: 25 - protein, 29 aa, Artificial Sequence
  source: /note=“Description of Artificial Sequence Synthetic peptide hybrid of human peptides”
SEGTFTSDYSKLLEEEAVRLFIEWLVKGP

SEQ ID NO: 26 - protein, 30 aa, Artificial Sequence
  source: /note=“Description of Artificial Sequence Synthetic peptide hybrid of human peptides”
YSEGTFTSDYSKLLDEEAVRLFIEWLVKGP

SEQ ID NO: 27 - protein, 30 aa, Artificial Sequence
  source: /note=“Description of Artificial Sequence Synthetic peptide hybrid of human peptides”
YSEGTFTSDYSKLLEKEAVRLFIEWLVKGP

SEQ ID NO: 28 - protein, 30 aa, Artificial Sequence
  source: /note=“Description of Artificial Sequence Synthetic peptide hybrid of human peptides”
YSEGTFTSDYSKLLEEEAVQLFIEWLVKGP

SEQ ID NO: 29 - protein, 30 aa, Artificial Sequence
  source: /note=“Description of Artificial Sequence Synthetic peptide hybrid of human peptides”
YSEGTFTSDYSKLLEEEAVRLFVEWLVKGP

SEQ ID NO: 30 - protein, 30 aa, Artificial Sequence
  source: /note=“Description of Artificial Sequence Synthetic peptide hybrid of human peptides”
YSEGTFTSDYSKLLEEEAVRLFIEWLLKGP

SEQ ID NO: 31 - protein, 30 aa, Artificial Sequence
  source: /note=“Description of Artificial Sequence Synthetic peptide hybrid of human peptides”
YSEGTFTSDYSKLLEEEAVRLFIEWLVKQP

SEQ ID NO: 32 - protein, 30 aa, Artificial Sequence
  source: /note=“Description of Artificial Sequence Synthetic peptide hybrid of human peptides”
YSEGTFTSDYSKLLEEEAVRLFIEWLVKGK

SEQ ID NO: 33 - protein, 29 aa, Artificial Sequence
  source: /note=“Description of Artificial Sequence Synthetic peptide hybrid of human peptides”
HGEGTFTSDVSSYMERQAIDEFIAWLLAG

SEQ ID NO: 34 - protein, 30 aa, Artificial Sequence
  source: /note=“Description of Artificial Sequence Synthetic peptide hybrid of human peptides”
HGEGTFTSDVSSYMERQAIDEFIAWLLKGG

SEQ ID NO: 35 - protein, 29 aa, Artificial Sequence
  source: /note=“Description of Artificial Sequence Synthetic peptide hybrid of human peptides”
YSEGTFTSDVSSYMERQAIDEFIAWLLAG

SEQ ID NO: 36 - protein, 30 aa, Artificial Sequence
  source: /note=“Description of Artificial Sequence Synthetic peptide hybrid of human peptides”
YSEGTFTSDVSSYMERQAIDEFIAWLLKGG

SEQ ID NO: 37 - protein, 30 aa, Artificial Sequence
  source: /note=“Description of Artificial Sequence Synthetic peptide hybrid of human peptides”
YSEGTFTSDYSSYMERQAIDEFIAWLLKGG

SEQ ID NO: 38 - protein, 30 aa, Artificial Sequence
  source: /note=“Description of Artificial Sequence Synthetic peptide hybrid of human peptides”
YSEGTFTSDVSKLLERQAIDEFIAWLLKGG

SEQ ID NO: 39 - protein, 30 aa, Artificial Sequence
  source: /note=“Description of Artificial Sequence Synthetic peptide hybrid of human peptides”
YSEGTFTSDVSIAMDRQAIDEFIAWLLKGG

SEQ ID NO: 40 - protein, 30 aa, Artificial Sequence
  source: /note=“Description of Artificial Sequence Synthetic peptide hybrid of human peptides”
YSEGTFTSDVSSYMEEEAIDEFIAWLLKGG

SEQ ID NO: 41 - protein, 30 aa, Artificial Sequence
  source: /note=“Description of Artificial Sequence Synthetic peptide hybrid of human peptides”
YSEGTFTSDVSSYMERQAIDEFVNDLLKGG

SEQ ID NO: 42 - protein, 30 aa, Artificial Sequence
  source: /note=“Description of Artificial Sequence Synthetic peptide hybrid of human peptides”
YSEGTFTSDVSSYMERQAIDEFIAWLLGGG

SEQ ID NO: 43 - protein, 30 aa, Artificial Sequence
  source: /note=“Description of Artificial Sequence Synthetic peptide hybrid of human peptides”
YSEGTFTSDVSSYMERQAIDEFIAWLLAQK

SEQ ID NO: 44 - protein, 29 aa, Artificial Sequence
  source: /note=“Description of Artificial Sequence Synthetic peptide hybrid of human peptides”
YSEGTFTSDVSSYMERQAISEFIAWLLAG

SEQ ID NO: 45 - protein, 30 aa, Artificial Sequence
  source: /note=“Description of Artificial Sequence Synthetic peptide hybrid of human peptides”
YSEGTFTSDVSSYMERQAISEFIAWLLKGP

SEQ ID NO: 46 - protein, 30 aa, Artificial Sequence
  source: /note=“Description of Artificial Sequence Synthetic peptide hybrid of human peptides”
HGEGTFTSDVSSYMEAQSIREFIAWLVKGP

SEQ ID NO: 47 - protein, 29 aa, Artificial Sequence
  source: /note=“Description of Artificial Sequence Synthetic peptide hybrid of human peptides”
HGEGTFTSDVSSYMEAQKIDEFIAWLVKG

SEQ ID NO: 48 - protein, 29 aa, Artificial Sequence
  source: /note=“Description of Artificial Sequence Synthetic peptide hybrid of human peptides”
HGEGTFTSDVSSYMESQLVREFIAWLVKG

SEQ ID NO: 49 - protein, 29 aa, Artificial Sequence
  source: /note=“Description of Artificial Sequence Synthetic peptide hybrid of human peptides”
HGEGTFTSDVSSYMESQLVDEFIAWLVKG

SEQ ID NO: 50 - protein, 29 aa, Artificial Sequence
  source: /note=“Description of Artificial Sequence Synthetic peptide hybrid of human peptides”
YSEGTFTSDVSSYLEGQAAKEFIAWLLAG

SEQ ID NO: 51 - protein, 30 aa, Artificial Sequence
  source: /note=“Description of Artificial Sequence Synthetic peptide hybrid of human peptides”
HGEGTFISDYSIAMDKIHQQDFVNWLLAQK

SEQ ID NO: 52 - protein, 29 aa, Artificial Sequence
  source: /note=“Description of Artificial Sequence Synthetic peptide hybrid of human peptides”
HGEGTFTSDVSSYLEGQAAKEFIAWLLAG

SEQ ID NO: 53 - protein, 29 aa, Artificial Sequence
  source: /note=“Description of Artificial Sequence Synthetic peptide hybrid of human peptides”
YSEGTFTSDVSSYLEEQAAKEFIAWLLAG

SEQ ID NO: 54 - protein, 30 aa, Artificial Sequence
  source: /note=“Description of Artificial Sequence Synthetic peptide hybrid of human peptides”
YSEGTFTSDVSSYLEGQAAKEFIAWLVAGG

SEQ ID NO: 55 - protein, 30 aa, Artificial Sequence
  source: /note=“Description of Artificial Sequence Synthetic peptide hybrid of human peptides”
HGEGTFTSDVSSYLEEEAAKEFIAWLVAGG

SEQ ID NO: 56 - protein, 39 aa, Artificial Sequence
  source: /note=“Description of Artificial Sequence Synthetic peptide hybrid of human peptides”
YSEGTFTSDLSKQMEEEAVRLFIEWLKNGGPSSGAPPPS

SEQ ID NO: 57 - protein, 30 aa, Artificial Sequence
  source: /note=“Description of Artificial Sequence Synthetic peptide hybrid of human peptides”
YSEGTFTSDVSSYLEEEAVREFIEWLLAGG

SEQ ID NO: 58 - protein, 30 aa, Artificial Sequence
  source: /note=“Description of Artificial Sequence Synthetic peptide hybrid of human peptides”
YSQGTFTSDYSKILEEEAVRLFIEWLVKGP

SEQ ID NO: 59 - protein, 29 aa, Artificial Sequence
  source: /note=“Description of Artificial Sequence Synthetic peptide hybrid of human peptides”
HGEGTFTSDVSSYLEGQAAKEFIAWLVKG

SEQ ID NO: 60 - protein, 29 aa, Artificial Sequence
  source: /note=“Description of Artificial Sequence Synthetic peptide hybrid of human peptides”
HGEGTFTSDVSSYLEEQAAKEFIAWLVKG

SEQ ID NO: 61 - protein, 30 aa, Artificial Sequence
  source: /note=“Description of Artificial Sequence Synthetic peptide hybrid of human peptides”
HGEGTFTSDVSSYLEEQAAKEFIAWLVKGG

SEQ ID NO: 62 - protein, 39 aa, Artificial Sequence
  source: /note=“Description of Artificial Sequence Synthetic peptide hybrid of human peptides”
HGEGTFTSDLSKQMEEEAVRLFIEWLKNGGPSSGAPPPS

SEQ ID NO: 63 - protein, 30 aa, Artificial Sequence
  source: /note=“Description of Artificial Sequence Synthetic peptide hybrid of human peptides”
YSEGTFISDYSIAMDKIHQQDFVNWLLAQK

SEQ ID NO: 64 - protein, 30 aa, Artificial Sequence; identical to SEQ ID NO: 63
  source: /note=“Description of Artificial Sequence Synthetic peptide hybrid of human peptides”

SEQ ID NO: 65 - protein, 42 aa, Artificial Sequence
  source: /note=“Description of Artificial Sequence Synthetic peptide hybrid of human peptides”
YAEGTFISDYSIAMDKIHQQDFVNWLLAQKGKKNDWKHNITQ

SEQ ID NO: 66 - protein, 22 aa, Artificial Sequence
  source: /note=“Description of Artificial Sequence Synthetic Linker peptide”
GGGGGSGGGGSGGGGSGGGGSA

SEQ ID NO: 67 - protein, 22 aa, Artificial Sequence
  source: /note=“Description of Artificial Sequence Synthetic Linker peptide ”
APPGGSGGGGSGGGGSGGGGSA

SEQ ID NO: 68 - protein, 23 aa, Artificial Sequence
  source: /note=“Description of Artificial Sequence Synthetic Linker peptide”
GTGGGGSGGGGSGGGGSGGGGSA

SEQ ID NO: 69 - protein, 22 aa, Artificial Sequence
  source: /note=“Description of Artificial Sequence Synthetic Linker peptide”
GGGGGSGGGGSGGGGSGGGGSA

SEQ ID NO: 70 - protein, 7 aa, Artificial Sequence
  source: /note=“Description of Artificial Sequence Synthetic Linker peptide”
GGGGGSA

SEQ ID NO: 71 - protein, 12 aa, Artificial Sequence
  source: /note=“Description of Artificial Sequence Synthetic Linker peptide”
GGGGGSGGGGSA

SEQ ID NO: 72 - protein, 17 aa, Artificial Sequence
  source: /note=“Description of Artificial Sequence Synthetic Linker peptide”
GGGGGSGGGGSGGGGSA

SEQ ID NO: 73 - protein, 22 aa, Artificial Sequence
  source: /note=“Description of Artificial Sequence Synthetic Linker peptide”
GKGGGSGGGGSGGGGSGGGGSA

SEQ ID NO: 74 - protein, 21 aa, Artificial Sequence
  source: /note=“Description of Artificial Sequence Synthetic Linker peptide”
GGSGSTASSGSGSATGGGGAA

SEQ ID NO: 75 - protein, 24 aa, Artificial Sequence
  source: /note=“Description of Artificial Sequence Synthetic Linker peptide”
AAAGGSGSTASSGSGSATGGGGAA

SEQ ID NO: 76 - protein, 24 aa, Artificial Sequence
  source: /note=“Description of Artificial Sequence Synthetic Linker peptide”
APPGGSGSTASSGSGSATGGGGAA

SEQ ID NO: 77 - protein, 17 aa, Artificial Sequence
  source: /note=“Description of Artificial Sequence Synthetic Hinge peptide”
ESKYGPPCPPCPAPEAA

SEQ ID NO: 78 - protein, 12 aa, Artificial Sequence
  source: /note=“Description of Artificial Sequence Synthetic Hinge peptide”
THTCPPCPAPEF

SEQ ID NO: 79 - protein, 7 aa, Artificial Sequence
  source: /note=“Description of Artificial Sequence Synthetic Hinge peptide”
THTCPPC

SEQ ID NO: 80 - protein, 9 aa, Artificial Sequence
  source: /note=“Description of Artificial Sequence Synthetic Hinge peptide”
CPPCPAPEF

SEQ ID NO: 81 - protein, 12 aa, Artificial Sequence
  source: /note=“Description of Artificial Sequence Synthetic Hinge peptide”
TYTCPPCPAPEF

SEQ ID NO: 82 - protein, 12 aa, Artificial Sequence
  source: /note=“Description of Artificial Sequence Synthetic Hinge peptide”
TSTCPPCPAPEF

SEQ ID NO: 83 - protein, 11 aa, Artificial Sequence
  source: /note=“Description of Artificial Sequence Synthetic Hinge peptide”
PPCPPCPAPEF

SEQ ID NO: 84 - protein, 16 aa, Artificial Sequence
  source: /note=“Description of Artificial Sequence Synthetic Hinge peptide”
ESKYGPPCPPCPAPEF

SEQ ID NO: 85 - protein, 4 aa, Artificial Sequence
  source: /note=“Description of Artificial Sequence Synthetic Hinge peptide”
APEF

SEQ ID NO: 86 - protein, 11 aa, Artificial Sequence
  source: /note=“Description of Artificial Sequence Synthetic Hinge peptide”
ESKYGPPCPPC

SEQ ID NO: 87 - protein, 13 aa, Artificial Sequence
  source: /note=“Description of Artificial Sequence Synthetic Hinge peptide”
THTCPPCPAPELL

SEQ ID NO: 88 - protein, 65 aa, Artificial Sequence
  source: /note=“Description of Artificial Sequence Synthetic Peptide-linker-hinge polypeptide”
YSEGTFTSDYSKLLEEEAVRLFIEWLLAGAAAGGSGSTASSGSGSATGGGGAATHTCPPCPAPEF

SEQ ID NO: 89 - protein, 65 aa, Artificial Sequence
  source: /note=“Description of Artificial Sequence Synthetic Peptide-linker-hinge polypeptide”
YSEGTFTSDYSKLLEEEAVRLFIEWLLAGAPPGGSGSTASSGSGSATGGGGAATHTCPPCPAPEF

SEQ ID NO: 90 - protein, 63 aa, Artificial Sequence
  source: /note=“Description of Artificial Sequence Synthetic Peptide-linker-hinge polypeptide”
YSEGTFTSDYSKLLEEEAVRLFIEWLLAGGGGGGSGGGGSGGGGSGGGGSATHTCPPCPAPEF

SEQ ID NO: 91 - protein, 63 aa, Artificial Sequence
  source: /note=“Description of Artificial Sequence Synthetic Peptide-linker-hinge polypeptide”
YSEGTFTSDYSKLLEEEAVRLFIEWLLAGAPPGGSGGGGSGGGGSGGGGSATHTCPPCPAPEF

SEQ ID NO: 92 - protein, 65 aa, Artificial Sequence; identical to SEQ ID NO: 88
  source: /note=“Description of Artificial Sequence Synthetic Peptide-linker-hinge polypeptide”

SEQ ID NO: 93 - protein, 65 aa, Artificial Sequence
  source: /note=“Description of Artificial Sequence Synthetic Peptide-linker-hinge polypeptide”
YSEGTFTSDVSKLLEEEAVRLFIEWLLAGAPPGGSGSTASSGSGSATGGGGAATHTCPPCPAPEF

SEQ ID NO: 94 - protein, 65 aa, Artificial Sequence
  source: /note=“Description of Artificial Sequence Synthetic Peptide-linker-hinge polypeptide”
YSEGTFTSDYSKLLDEEAVRLFIEWLLAGAPPGGSGSTASSGSGSATGGGGAATHTCPPCPAPEF

SEQ ID NO: 95 - protein, 65 aa, Artificial Sequence
  source: /note=“Description of Artificial Sequence Synthetic Peptide-linker-hinge polypeptide”
YSEGTFTSDYSKLLEEEAVRLFIEWLVKGAAAGGSGSTASSGSGSATGGGGAATHTCPPCPAPEF

SEQ ID NO: 96 - protein, 65 aa, Artificial Sequence
  source: /note=“Description of Artificial Sequence Synthetic Peptide-linker-hinge polypeptide”
YSEGTFTSDYSKLLEEEAVRLFIEWLLAGNGTGGGGSGGGGSGGGGSGGGGSATHTCPPCPAPEF

SEQ ID NO: 97 - protein, 69 aa, Artificial Sequence
  source: /note=“Description of Artificial Sequence Synthetic Peptide-linker-hinge polypeptide”
YSEGTFTSDYSKLLEEEAVRLFIEWLLAGGHNITQGGGGGSGGGGSGGGGSGGGGSATHTCPPCPAPEF

SEQ ID NO: 98 - protein, 66 aa, Artificial Sequence
  source: /note=“Description of Artificial Sequence Synthetic Peptide-linker-hinge polypeptide”
YSEGTFTSDYSKLLEEEAVRLFIEWLLAGGAAAGGSGSTASSGSGSATGGGGAATHTCPPCPAPEF

SEQ ID NO: 99 - protein, 66 aa, Artificial Sequence
  source: /note=“Description of Artificial Sequence Synthetic Peptide-linker-hinge polypeptide”
YSEGTFTSDYSKLLEEEAVRLFIEWLLAGPAAAGGSGSTASSGSGSATGGGGAATHTCPPCPAPEF

SEQ ID NO: 100 - protein, 74 aa, Artificial Sequence
  source: /note=“Description of Artificial Sequence Synthetic Peptide-linker-hinge polypeptide”
YSEGTFTSDYSKLLEEEAVRLFIEWLLAGPSSGAPPPSAAAGGSGSTASSGSGSATGGGGAATHTCPPCPAPEF

SEQ ID NO: 101 - protein, 66 aa, Artificial Sequence
  source: /note=“Description of Artificial Sequence Synthetic Peptide-linker-hinge polypeptide”
YSEGTFTSDYSKLLEEEAVRLFIEWLLAQKAAAGGSGSTASSGSGSATGGGGAATHTCPPCPAPEF

SEQ ID NO: 102 - protein, 66 aa, Artificial Sequence
  source: /note=“Description of Artificial Sequence Synthetic Peptide-linker-hinge polypeptide”
YSEGTFTSDYSKLLEEEAVRLFIEWLKNGGAAAGGSGSTASSGSGSATGGGGAATHTCPPCPAPEF

SEQ ID NO: 103 - protein, 75 aa, Artificial Sequence
  source: /note=“Description of Artificial Sequence Synthetic Peptide-linker-hinge polypeptide”
YSEGTFTSDYSKLLEEEAVRLFIEWLKNGGPSSGAPPPSAAAGGSGSTASSGSGSATGGGGAATHTCPPCPAPEF

SEQ ID NO: 104 - protein, 64 aa, Artificial Sequence
  source: /note=“Description of Artificial Sequence Synthetic Peptide-linker-hinge polypeptide”
YSEGTFTSDYSKLLEEEAVRLFIEWLKNGTGGGGGSGGGGSGGGGSGGGGSATHTCPPCPAPEF

SEQ ID NO: 105 - protein, 66 aa, Artificial Sequence
  source: /note=“Description of Artificial Sequence Synthetic Peptide-linker-hinge polypeptide”
YSEGTFTSDYSKLLEEEAVRLFIEWLVKGPAAAGGSGSTASSGSGSATGGGGAATHTCPPCPAPEF

SEQ ID NO: 106 - protein, 66 aa, Artificial Sequence
  source: /note=“Description of Artificial Sequence Synthetic Peptide-linker-hinge polypeptide”
YSEGTFTSDYSKLLEEEAVRLFIEWLVKGPAPPGGSGSTASSGSGSATGGGGAATHTCPPCPAPEF

SEQ ID NO: 107 - protein, 66 aa, Artificial Sequence
  source: /note=“Description of Artificial Sequence Synthetic Peptide-linker-hinge polypeptide”
YSQGTFTSDYSKLLEEEAVRLFIEWLVKGPAPPGGSGSTASSGSGSATGGGGAATHTCPPCPAPEF

SEQ ID NO: 108 - protein, 49 aa, Artificial Sequence
  source: /note=“Description of Artificial Sequence Synthetic Peptide-linker-hinge polypeptide”
YSEGTFTSDYSKLLEEEAVRLFIEWLVKGPGGGGGSATHTCPPCPAPEF

SEQ ID NO: 109 - protein, 54 aa, Artificial Sequence
  source: /note=“Description of Artificial Sequence Synthetic Peptide-linker-hinge polypeptide”
YSEGTFTSDYSKLLEEEAVRLFIEWLVKGPGGGGGSGGGGSATHTCPPCPAPEF

SEQ ID NO: 110 - protein, 59 aa, Artificial Sequence
  source: /note=“Description of Artificial Sequence Synthetic Peptide-linker-hinge polypeptide”
YSEGTFTSDYSKLLEEEAVRLFIEWLVKGPGGGGGSGGGGSGGGGSATHTCPPCPAPEF

SEQ ID NO: 111 - protein, 64 aa, Artificial Sequence
  source: /note=“Description of Artificial Sequence Synthetic Peptide-linker-hinge polypeptide”
YSEGTFTSDYSKLLEEEAVRLFIEWLVKGPGGGGGSGGGGSGGGGSGGGGSATHTCPPCPAPEF

SEQ ID NO: 112 - protein, 63 aa, Artificial Sequence
  source: /note=“Description of Artificial Sequence Synthetic Peptide-linker-hinge polypeptide”
YSEGTFTSDYSKLLEEEAVRLFIEWLVKGPAAAGGSGSTASSGSGSATGGGGAACPPCPAPEF

SEQ ID NO: 113 - protein, 66 aa, Artificial Sequence
  source: /note=“Description of Artificial Sequence Synthetic Peptide-linker-hinge polypeptide”
YSEGTFTSDLSKLLEEEAVRLFIEWLVKGPAAAGGSGSTASSGSGSATGGGGAATHTCPPCPAPEF

SEQ ID NO: 114 - protein, 66 aa, Artificial Sequence
  source: /note=“Description of Artificial Sequence Synthetic Peptide-linker-hinge polypeptide”
YSEGTFTSDYSSYMEEEAVRLFIEWLVKGPAAAGGSGSTASSGSGSATGGGGAATHTCPPCPAPEF

SEQ ID NO: 115 - protein, 66 aa, Artificial Sequence
  source: /note=“Description of Artificial Sequence Synthetic Peptide-linker-hinge polypeptide”
YSEGTFTSDYSIAMDEEAVRLFIEWLVKGPAAAGGSGSTASSGSGSATGGGGAATHTCPPCPAPEF

SEQ ID NO: 116 - protein, 66 aa, Artificial Sequence
  source: /note=“Description of Artificial Sequence Synthetic Peptide-linker-hinge polypeptide”
YSEGTFTSDYSKLLEGQAAKLFIEWLVKGPAAAGGSGSTASSGSGSATGGGGAATHTCPPCPAPEF

SEQ ID NO: 117 - protein, 66 aa, Artificial Sequence
  source: /note=“Description of Artificial Sequence Synthetic Peptide-linker-hinge polypeptide”
YSEGTFTSDYSKLLEKIHQQLFIEWLVKGPAAAGGSGSTASSGSGSATGGGGAATHTCPPCPAPEF

SEQ ID NO: 118 - protein, 66 aa, Artificial Sequence
  source: /note=“Description of Artificial Sequence Synthetic Peptide-linker-hinge polypeptide”
YSEGTFTSDYSKLLEEEAQRLFIEWLVKGPAAAGGSGSTASSGSGSATGGGGAATHTCPPCPAPEF

SEQ ID NO: 119 - protein, 66 aa, Artificial Sequence
  source: /note=“Description of Artificial Sequence Synthetic Peptide-linker-hinge polypeptide”
YSEGTFTSDYSKLLEEEAVREFIAWLVKGPAAAGGSGSTASSGSGSATGGGGAATHTCPPCPAPEF

SEQ ID NO: 120 - protein, 66 aa, Artificial Sequence
  source: /note=“Description of Artificial Sequence Synthetic Peptide-linker-hinge polypeptide”
YSEGTFTSDYSKLLEEEAVRDFVNWLVKGPAAAGGSGSTASSGSGSATGGGGAATHTCPPCPAPEF

SEQ ID NO: 121 - protein, 64 aa, Artificial Sequence
  source: /note=“Description of Artificial Sequence Synthetic Peptide-linker-hinge polypeptide”
YSEGTFTSDYSKLLEEEAVRLFIEWLVKGPGGGGGSGGGGSGGGGSAESKYGPPCPPCPAPEAA

SEQ ID NO: 122 - protein, 63 aa, Artificial Sequence
  source: /note=“Description of Artificial Sequence Synthetic Peptide-linker-hinge polypeptide”
SEGTFTSDYSKLLEEEAVRLFIEWLVKGPGGGGGSGGGGSGGGGSGGGGSATHTCPPCPAPEF

SEQ ID NO: 123 - protein, 64 aa, Artificial Sequence
  source: /note=“Description of Artificial Sequence Synthetic Peptide-linker-hinge polypeptide”
YSEGTFTSDYSKLLDEEAVRLFIEWLVKGPGGGGGSGGGGSGGGGSGGGGSATHTCPPCPAPEF

SEQ ID NO: 124 - protein, 64 aa, Artificial Sequence
  source: /note=“Description of Artificial Sequence Synthetic Peptide-linker-hinge polypeptide”
YSEGTFTSDYSKLLEKEAVRLFIEWLVKGPGGGGGSGGGGSGGGGSGGGGSATHTCPPCPAPEF

SEQ ID NO: 125 - protein, 64 aa, Artificial Sequence
  source: /note=“Description of Artificial Sequence Synthetic Peptide-linker-hinge polypeptide”
YSEGTFTSDYSKLLEEEAVQLFIEWLVKGPGGGGGSGGGGSGGGGSGGGGSATHTCPPCPAPEF

SEQ ID NO: 126 - protein, 64 aa, Artificial Sequence
  source: /note=“Description of Artificial Sequence Synthetic Peptide-linker-hinge polypeptide”
YSEGTFTSDYSKLLEEEAVRLFVEWLVKGPGGGGGSGGGGSGGGGSGGGGSATHTCPPCPAPEF

SEQ ID NO: 127 - protein, 64 aa, Artificial Sequence
  source: /note=“Description of Artificial Sequence Synthetic Peptide-linker-hinge polypeptide”
YSEGTFTSDYSKLLEEEAVRLFIEWLLKGPGGGGGSGGGGSGGGGSGGGGSATHTCPPCPAPEF

SEQ ID NO: 128 - protein, 64 aa, Artificial Sequence
  source: /note=“Description of Artificial Sequence Synthetic Peptide-linker-hinge polypeptide”
YSEGTFTSDYSKLLEEEAVRLFIEWLVKQPGGGGGSGGGGSGGGGSGGGGSATHTCPPCPAPEF

SEQ ID NO: 129 - protein, 64 aa, Artificial Sequence
  source: /note=“Description of Artificial Sequence Synthetic Peptide-linker-hinge polypeptide”
YSEGTFTSDYSKLLEEEAVRLFIEWLVKGKGGGGGSGGGGSGGGGSGGGGSATHTCPPCPAPEF

SEQ ID NO: 130 - protein, 64 aa, Artificial Sequence
  source: /note=“Description of Artificial Sequence Synthetic Peptide-linker-hinge polypeptide”
YSEGTFTSDYSKLLEEEAVRLFIEWLVKGPGKGGGSGGGGSGGGGSGGGGSATHTCPPCPAPEF

SEQ ID NO: 131 - protein, 65 aa, Artificial Sequence
  source: /note=“Description of Artificial Sequence Synthetic Peptide-linker-hinge polypeptide”
HGEGTFTSDVSSYMERQAIDEFIAWLLAGAAAGGSGSTASSGSGSATGGGGAATHTCPPCPAPEF

SEQ ID NO: 132 - protein, 66 aa, Artificial Sequence
  source: /note=“Description of Artificial Sequence Synthetic Peptide-linker-hinge polypeptide”
HGEGTFTSDVSSYMERQAIDEFIAWLLKGGAAAGGSGSTASSGSGSATGGGGAATHTCPPCPAPEF

SEQ ID NO: 133 - protein, 65 aa, Artificial Sequence
  source: /note=“Description of Artificial Sequence Synthetic Peptide-linker-hinge polypeptide”
YSEGTFTSDVSSYMERQAIDEFIAWLLAGAAAGGSGSTASSGSGSATGGGGAATHTCPPCPAPEF

SEQ ID NO: 134 - protein, 66 aa, Artificial Sequence
  source: /note=“Description of Artificial Sequence Synthetic Peptide-linker-hinge polypeptide”
YSEGTFTSDVSSYMERQAIDEFIAWLLKGGAAAGGSGSTASSGSGSATGGGGAATHTCPPCPAPEF

SEQ ID NO: 135 - protein, 66 aa, Artificial Sequence
  source: /note=“Description of Artificial Sequence Synthetic Peptide-linker-hinge polypeptide”
YSEGTFTSDYSSYMERQAIDEFIAWLLKGGAAAGGSGSTASSGSGSATGGGGAATHTCPPCPAPEF

SEQ ID NO: 136 - protein, 66 aa, Artificial Sequence
  source: /note=“Description of Artificial Sequence Synthetic Peptide-linker-hinge polypeptide”
YSEGTFTSDVSKLLERQAIDEFIAWLLKGGAAAGGSGSTASSGSGSATGGGGAATHTCPPCPAPEF

SEQ ID NO: 137 - protein, 66 aa, Artificial Sequence
  source: /note=“Description of Artificial Sequence Synthetic Peptide-linker-hinge polypeptide”
YSEGTFTSDVSIAMDRQAIDEFIAWLLKGGAAAGGSGSTASSGSGSATGGGGAATHTCPPCPAPEF

SEQ ID NO: 138 - protein, 66 aa, Artificial Sequence
  source: /note=“Description of Artificial Sequence Synthetic Peptide-linker-hinge polypeptide”
YSEGTFTSDVSSYMEEEAIDEFIAWLLKGGAAAGGSGSTASSGSGSATGGGGAATHTCPPCPAPEF

SEQ ID NO: 139 - protein, 66 aa, Artificial Sequence
  source: /note=“Description of Artificial Sequence Synthetic Peptide-linker-hinge polypeptide”
YSEGTFTSDVSSYMERQAIDEFVNDLLKGGAAAGGSGSTASSGSGSATGGGGAATHTCPPCPAPEF

SEQ ID NO: 140 - protein, 66 aa, Artificial Sequence
  source: /note=“Description of Artificial Sequence Synthetic Peptide-linker-hinge polypeptide”
YSEGTFTSDVSSYMERQAIDEFIAWLLGGGAAAGGSGSTASSGSGSATGGGGAATHTCPPCPAPEF

SEQ ID NO: 141 - protein, 66 aa, Artificial Sequence
  source: /note=“Description of Artificial Sequence Synthetic Peptide-linker-hinge polypeptide”
YSEGTFTSDVSSYMERQAIDEFIAWLLAQKAAAGGSGSTASSGSGSATGGGGAATHTCPPCPAPEF

SEQ ID NO: 142 - protein, 65 aa, Artificial Sequence
  source: /note=“Description of Artificial Sequence Synthetic Peptide-linker-hinge polypeptide”
YSEGTFTSDVSSYMERQAISEFIAWLLAGAAAGGSGSTASSGSGSATGGGGAATHTCPPCPAPEF

SEQ ID NO: 143 - protein, 66 aa, Artificial Sequence
  source: /note=“Description of Artificial Sequence Synthetic Peptide-linker-hinge polypeptide”
YSEGTFTSDVSSYMERQAISEFIAWLLKGPAAAGGSGSTASSGSGSATGGGGAATHTCPPCPAPEF

SEQ ID NO: 144 - protein, 66 aa, Artificial Sequence
  source: /note=“Description of Artificial Sequence Synthetic Peptide-linker-hinge polypeptide”
HGEGTFTSDVSSYMEAQSIREFIAWLVKGPAPPGGSGSTASSGSGSATGGGGAATHTCPPCPAPEF

SEQ ID NO: 145 - protein, 65 aa, Artificial Sequence
  source: /note=“Description of Artificial Sequence Synthetic Peptide-linker-hinge polypeptide”
HGEGTFTSDVSSYMEAQKIDEFIAWLVKGAPPGGSGSTASSGSGSATGGGGAATHTCPPCPAPEF

SEQ ID NO: 146 - protein, 65 aa, Artificial Sequence
  source: /note=“Description of Artificial Sequence Synthetic Peptide-linker-hinge polypeptide”
HGEGTFTSDVSSYMESQLVDEFIAWLVKGAAAGGSGSTASSGSGSATGGGGAATHTCPPCPAPEF

SEQ ID NO: 147 - protein, 65 aa, Artificial Sequence
  source: /note=“Description of Artificial Sequence Synthetic Peptide-linker-hinge polypeptide”
HGEGTFTSDVSSYMESQLVREFIAWLVKGAAAGGSGSTASSGSGSATGGGGAATHTCPPCPAPEF

SEQ ID NO: 148 - protein, 65 aa, Artificial Sequence
  source: /note=“Description of Artificial Sequence Synthetic Peptide-linker-hinge polypeptide”
YSEGTFTSDVSSYLEGQAAKEFIAWLLAGAAAGGSGSTASSGSGSATGGGGAATHTCPPCPAPEF

SEQ ID NO: 149 - protein, 66 aa, Artificial Sequence
  source: /note=“Description of Artificial Sequence Synthetic Peptide-linker-hinge polypeptide”
HGEGTFISDYSIAMDKIHQQDFVNWLLAQKAAAGGSGSTASSGSGSATGGGGAATHTCPPCPAPEF

SEQ ID NO: 150 - protein, 65 aa, Artificial Sequence
  source: /note=“Description of Artificial Sequence Synthetic Peptide-linker-hinge polypeptide”
HGEGTFTSDVSSYLEGQAAKEFIAWLLAGAAAGGSGSTASSGSGSATGGGGAATHTCPPCPAPEF

SEQ ID NO: 151 - protein, 65 aa, Artificial Sequence
  source: /note=“Description of Artificial Sequence Synthetic Peptide-linker-hinge polypeptide”
YSEGTFTSDVSSYLEEQAAKEFIAWLLAGAAAGGSGSTASSGSGSATGGGGAATHTCPPCPAPEF

SEQ ID NO: 152 - protein, 66 aa, Artificial Sequence
  source: /note=“Description of Artificial Sequence Synthetic Peptide-linker-hinge polypeptide”
YSEGTFTSDVSSYLEGQAAKEFIAWLVAGGAPPGGSGSTASSGSGSATGGGGAATHTCPPCPAPEF

SEQ ID NO: 153 - protein, 66 aa, Artificial Sequence
  source: /note=“Description of Artificial Sequence Synthetic Peptide-linker-hinge polypeptide”
HGEGTFTSDVSSYLEEEAAKEFIAWLVAGGAAAGGSGSTASSGSGSATGGGGAATHTCPPCPAPEF

SEQ ID NO: 154 - protein, 75 aa, Artificial Sequence
  source: /note=“Description of Artificial Sequence Synthetic Peptide-linker-hinge polypeptide”
YSEGTFTSDLSKQMEEEAVRLFIEWLKNGGPSSGAPPPSAAAGGSGSTASSGSGSATGGGGAATHTCPPCPAPEF

SEQ ID NO: 155 - protein, 66 aa, Artificial Sequence
  source: /note=“Description of Artificial Sequence Synthetic Peptide-linker-hinge polypeptide”
YSEGTFTSDVSSYLEEEAVREFIEWLLAGGAAAGGSGSTASSGSGSATGGGGAATHTCPPCPAPEF

SEQ ID NO: 156 - protein, 66 aa, Artificial Sequence
  source: /note=“Description of Artificial Sequence Synthetic Peptide-linker-hinge polypeptide”
YSQGTFTSDYSKILEEEAVRLFIEWLVKGPAAAGGSGSTASSGSGSATGGGGAATHTCPPCPAPEF

SEQ ID NO: 157 - protein, 65 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGAATHTCPPCPAPEF

SEQ ID NO: 158 - protein, 58 aa, Artificial Sequence
  source: /note=“Description of Artificial Sequence Synthetic Peptide-linker-hinge polypeptide”
HGEGTFTSDVSSYLEEQAAKEFIAWLVKGGGGGGSGGGGSGGGGSGGGGSATHTCPPC

SEQ ID NO: 159 - protein, 65 aa, Artificial Sequence
  source: /note=“Description of Artificial Sequence Synthetic Peptide-linker-hinge polypeptide”
HGEGTFTSDVSSYLEEQAAKEFIAWLVKGAAAGGSGSTASSGSGSATGGGGAATHTCPPCPAPEF

SEQ ID NO: 160 - protein, 63 aa, Artificial Sequence
  source: /note=“Description of Artificial Sequence Synthetic Peptide-linker-hinge polypeptide”
HGEGTFTSDVSSYLEGQAAKEFIAWLVKGGGGGGSGGGGSGGGGSGGGGSATHTCPPCPAPEF

SEQ ID NO: 161 - protein, 62 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGAACPPCPAPEF

SEQ ID NO: 162 - protein, 66 aa, Artificial Sequence
  source: /note=“Description of Artificial Sequence Synthetic Peptide-linker-hinge polypeptide”
HGEGTFTSDVSSYLEGQAAKEFIAWLVKGAPPGGGGGSGGGGSGGGGSGGGGSATHTCPPCPAPEF

SEQ ID NO: 163 - protein, 65 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGAATYTCPPCPAPEF

SEQ ID NO: 164 - protein, 65 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGAATSTCPPCPAPEF

SEQ ID NO: 165 - protein, 60 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGCPPCPAPEF

SEQ ID NO: 166 - protein, 62 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGPPCPPCPAPEF

SEQ ID NO: 167 - protein, 67 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGESKYGPPCPPCPAPEF

SEQ ID NO: 168 - protein, 55 aa, Artificial Sequence
  source: /note=“Description of Artificial Sequence Synthetic Peptide-linker-hinge polypeptide”
HGEGTFTSDVSSYLEGQAAKEFIAWLVKGAAAGGSGSTASSGSGSATGGGGAPEF

SEQ ID NO: 169 - protein, 66 aa, Artificial Sequence
  source: /note=“Description of Artificial Sequence Synthetic Peptide-linker-hinge polypeptide”
HGEGTFTSDVSSYLEGQAAKEFIAWLVKGGAAAGGSGSTASSGSGSATGGGGAATHTCPPCPAPEF

SEQ ID NO: 170 - protein, 65 aa, Artificial Sequence; identical to SEQ ID NO: 157
  source: /note=“Description of Artificial Sequence Synthetic Peptide-linker-hinge polypeptide”

SEQ ID NO: 171 - protein, 57 aa, Artificial Sequence
  source: /note=“Description of Artificial Sequence Synthetic Peptide-linker-hinge polypeptide”
HGEGTFTSDVSSYLEGQAAKEFIAWLVKGGGGGGSGGGGSGGGGSAESKYGPPCPPC

SEQ ID NO: 172 - protein, 75 aa, Artificial Sequence
  source: /note=“Description of Artificial Sequence Synthetic Peptide-linker-hinge polypeptide”
HGEGTFTSDLSKQMEEEAVRLFIEWLKNGGPSSGAPPPSAAAGGSGSTASSGSGSATGGGGAATHTCPPCPAPEF

SEQ ID NO: 173 - protein, 66 aa, Artificial Sequence
  source: /note=“Description of Artificial Sequence Synthetic Peptide-linker-hinge polypeptide”
YSEGTFISDYSIAMDKIHQQDFVNWLLAQKAAAGGSGSTASSGSGSATGGGGAATHTCPPCPAPEF

SEQ ID NO: 174 - protein, 67 aa, Artificial Sequence
  source: /note=“Description of Artificial Sequence Synthetic Peptide-linker-hinge polypeptide”
YSEGTFISDYSIAMDKIHQQDFVNWLLAQKAPPGGGGGSGGGGSGGGGSGGGGSATHTCPPCPAPEF

SEQ ID NO: 175 - protein, 79 aa, Artificial Sequence
  source: /note=“Description of Artificial Sequence Synthetic Peptide-linker-hinge polypeptide”
YAEGTFISDYSIAMDKIHQQDFVNWLLAQKGKKNDWKHNITQAAAGGSGSTASSGSGSATGGGGAATHTCPPCPAPELL

SEQ ID NO: 176 - protein, 30 aa, Homo sapiens
HAEGTFTSDVSSYLEGQAAKEFIAWLVKGR

SEQ ID NO: 177 - protein, 31 aa, Homo sapiens
HAEGTFTSDVSSYLEGQAAKEFIAWLVKGRG

SEQ ID NO: 178 - protein, 42 aa, Homo sapiens
YAEGTFISDYSIAMDKIHQQDFVNWLAQKGKKKNDWKHNITQ

SEQ ID NO: 179 - protein, 22 aa, Artificial Sequence
  source: /note=“Description of Artificial Sequence Synthetic Peptide linker”
GGGGGSGGGGSGGGGSGGGGSA

SEQ ID NO: 180 - protein, 18 aa, Artificial Sequence
  source: /note=“Description of Artificial Sequence Synthetic Peptide linker”
GGGGGGGGGGGGGGGGGA

SEQ ID NO: 181 - protein, 212 aa, Homo sapiens
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 212 aa, Homo sapiens
AGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 183 - protein, 213 aa, Homo sapiens
EGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 212 aa, Homo sapiens
GGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 213 aa, Homo sapiens
QGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 213 aa, Homo sapiens
EGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 30 aa, Artificial Sequence
  source: /note=“Description of Artificial Sequence Synthetic GLP-1 analog polypeptide”
HGEGTFTSDLSKQMEEEAVRLFIEWLKNGG

SEQ ID NO: 188 - protein, 30 aa, Homo sapiens
YAEGTFISDYSIAMDKIHQQDFVNWLLAQK

SEQ ID NO: 189 - protein, 30 aa, Artificial Sequence
  source: /note=“Description of Artificial Sequence Synthetic GLP-1 Analog polypeptide”
HGEGTFTSDVSSYLEEQAAKEFIAWLVKGR

SEQ ID NO: 190 - protein, 30 aa, Artificial Sequence
  source: /note=“Description of Artificial Sequence Synthetic polypeptide hybrid of human peptides”
HGEGTFTSDVSSYMERQAIDEFIAWLLAGG

SEQ ID NO: 191 - protein, 29 aa, Artificial Sequence
  source: /note=“Description of Artificial Sequence Synthetic peptide”
YSEGTFTSDYSKLLEEEAVRLFIEWLLAG

SEQ ID NO: 192 - protein, 30 aa, Artificial Sequence
  source: /note=“Description of Artificial Sequence Synthetic polypeptide”
YSEGTFTSDYSKLLEEEHVRLFIEWLVKGP

SEQ ID NO: 193 - protein, 30 aa, Artificial Sequence; identical to SEQ ID NO: 15
  source: /note=“Description of Artificial Sequence Synthetic polypeptide”

SEQ ID NO: 194 - protein, 30 aa, Artificial Sequence; identical to SEQ ID NO: 11
  source: /note=“Description of Artificial Sequence Synthetic polypeptide”

SEQ ID NO: 195 - protein, 30 aa, Artificial Sequence; identical to SEQ ID NO: 15
  source: /note=“Description of Artificial Sequence Synthetic polypeptide”

SEQ ID NO: 196 - protein, 30 aa, Artificial Sequence
  source: /note=“Description of Artificial Sequence Synthetic polypeptide”
YSEGTFTSDYSKLLEKEAVRLFIEWLLKGP

SEQ ID NO: 197 - protein, 30 aa, Artificial Sequence
  source: /note=“Description of Artificial Sequence Synthetic polypeptide”
YSEGTFTSDYSKLLEKEAQRLFIEWLLKGP

SEQ ID NO: 198 - protein, 30 aa, Artificial Sequence; identical to SEQ ID NO: 15
  source: /note=“Description of Artificial Sequence Synthetic polypeptide”

SEQ ID NO: 199 - protein, 39 aa, Artificial Sequence
  source: /note=“Description of Artificial Sequence Synthetic polypeptide”
YSEGTFTSDYSKLLEEEAVRLFIEWLVKGPPSSGAPPPS

SEQ ID NO: 200 - protein, 38 aa, Artificial Sequence
  source: /note=“Description of Artificial Sequence Synthetic polypeptide”
YSEGTFTSDYSKLLEEEAVRLFIEWLVKGPSSGAPPPS

SEQ ID NO: 201 - protein, 39 aa, Artificial Sequence
  source: /note=“Description of Artificial Sequence Synthetic polypeptide”
YSEGTFTSDYSKLLEEEAVRLFIEWLVKGGPSSGAPPPS

SEQ ID NO: 202 - protein, 30 aa, Artificial Sequence; identical to SEQ ID NO: 15
  source: /note=“Description of Artificial Sequence Synthetic polypeptide”

SEQ ID NO: 203 - protein, 30 aa, Artificial Sequence; identical to SEQ ID NO: 15
  source: /note=“Description of Artificial Sequence Synthetic polypeptide”

SEQ ID NO: 204 - protein, 30 aa, Artificial Sequence; identical to SEQ ID NO: 15
  source: /note=“Description of Artificial Sequence Synthetic polypeptide”

SEQ ID NO: 205 - protein, 30 aa, Artificial Sequence; identical to SEQ ID NO: 15
  source: /note=“Description of Artificial Sequence Synthetic polypeptide”

SEQ ID NO: 206 - protein, 30 aa, Artificial Sequence; identical to SEQ ID NO: 15
  source: /note=“Description of Artificial Sequence Synthetic polypeptide”

SEQ ID NO: 207 - protein, 30 aa, Artificial Sequence
  source: /note=“Description of Artificial Sequence Synthetic polypeptide”
YSEGTFTSDYSKLLEKEAERLFIEWLLKGP

SEQ ID NO: 208 - protein, 30 aa, Artificial Sequence; identical to SEQ ID NO: 15
  source: /note=“Description of Artificial Sequence Synthetic polypeptide”

SEQ ID NO: 209 - protein, 40 aa, Artificial Sequence
  source: /note=“Description of Artificial Sequence Synthetic polypeptide”
YSEGTFTSDYSKLLEEECVRLFIEWLVKGGPGGGAPPPGC

SEQ ID NO: 210 - protein, 40 aa, Artificial Sequence
  source: /note=“Description of Artificial Sequence Synthetic polypeptide”
YSEGTFTSDYSKLLEEEAVRCFIEWLVKGGPGGGAPPCGG

SEQ ID NO: 211 - protein, 30 aa, Artificial Sequence
  source: /note=“Description of Artificial Sequence Synthetic polypeptide”
YSEGTFTSDYSKLLEEEAVRCFIECLVKGP

SEQ ID NO: 212 - protein, 30 aa, Artificial Sequence
  source: /note=“Description of Artificial Sequence Synthetic polypeptide”
YSEGTFTSDYSKLLEEEAVRLFICWLVCGP

SEQ ID NO: 213 - protein, 39 aa, Artificial Sequence
  source: /note=“Description of Artificial Sequence Synthetic polypeptide”
YSEGTFTSDYSKLLEEEAVRLFIEWLLAGGPSSGAPPPS

SEQ ID NO: 214 - protein, 39 aa, Artificial Sequence
  source: /note=“Description of Artificial Sequence Synthetic polypeptide”
YSEGTFTSDYSKLLEEEAVRCFIEWLVKGGPSSGAPPCS

SEQ ID NO: 215 - protein, 29 aa, Artificial Sequence
  source: /note=“Description of Artificial Sequence Synthetic peptide”
YSEGTFTSDYSKLLEEEAVRLFIEWLLAG

SEQ ID NO: 216 - protein, 30 aa, Artificial Sequence
  source: /note=“Description of Artificial Sequence Synthetic polypeptide”
YSEGTFISDYSKLLEEEAVRDFVEWLKAGG

SEQ ID NO: 217 - protein, 29 aa, Artificial Sequence
  source: /note=“Description of Artificial Sequence Synthetic peptide”
YSEGTFTSDYSKLLEEEAVRLFIEWLLAG

SEQ ID NO: 218 - protein, 30 aa, Artificial Sequence; identical to SEQ ID NO: 15
  source: /note=“Description of Artificial Sequence Synthetic polypeptide”

SEQ ID NO: 219 - protein, 30 aa, Artificial Sequence; identical to SEQ ID NO: 15
  source: /note=“Description of Artificial Sequence Synthetic polypeptide”

SEQ ID NO: 220 - protein, 30 aa, Artificial Sequence; identical to SEQ ID NO: 15
  source: /note=“Description of Artificial Sequence Synthetic polypeptide”

SEQ ID NO: 221 - protein, 30 aa, Artificial Sequence; identical to SEQ ID NO: 15
  source: /note=“Description of Artificial Sequence Synthetic polypeptide”

SEQ ID NO: 222 - protein, 30 aa, Artificial Sequence; identical to SEQ ID NO: 8
  source: /note=“Description of Artificial Sequence Synthetic polypeptide”

SEQ ID NO: 223 - protein, 30 aa, Artificial Sequence; identical to SEQ ID NO: 8
  source: /note=“Description of Artificial Sequence Synthetic polypeptide”

SEQ ID NO: 224 - protein, 30 aa, Artificial Sequence; identical to SEQ ID NO: 8
  source: /note=“Description of Artificial Sequence Synthetic polypeptide”

SEQ ID NO: 225 - protein, 30 aa, Artificial Sequence
  source: /note=“Description of Artificial Sequence Synthetic polypeptide”
YSEGTFTSDYSKLLEKEAQRLFIEWLLAGG

SEQ ID NO: 226 - protein, 39 aa, Artificial Sequence; identical to SEQ ID NO: 62
  source: /note=“Description of Artificial Sequence Synthetic polypeptide”

SEQ ID NO: 227 - protein, 30 aa, Artificial Sequence
  source: /note=“Description of Artificial Sequence Synthetic polypeptide”
YSEGMHTSDFSKLLEEEAVRLFIEWLLAGG

SEQ ID NO: 228 - protein, 30 aa, Artificial Sequence
  source: /note=“Description of Artificial Sequence Synthetic polypeptide”
YSEGTHTSLFSKLLEEEAVRLFIEWLLAGG

SEQ ID NO: 229 - protein, 30 aa, Artificial Sequence
  source: /note=“Description of Artificial Sequence Synthetic polypeptide”
YSEGTFTSDFSKLLEEEAVRLFIEFLLAGG

SEQ ID NO: 230 - protein, 30 aa, Artificial Sequence
  source: /note=“Description of Artificial Sequence Synthetic polypeptide”
YSEGTFTSDFSKLLEEEAVRLFIEWLLAGG

SEQ ID NO: 231 - protein, 30 aa, Artificial Sequence
  source: /note=“Description of Artificial Sequence Synthetic polypeptide”
YSEGTFTSDYSKLLEEEAVRLFIEHLLAGG

SEQ ID NO: 232 - protein, 30 aa, Artificial Sequence
  source: /note=“Description of Artificial Sequence Synthetic polypeptide”
YSEGTFTSDYRILLEEEAVRLFIEWLLAGG

SEQ ID NO: 233 - protein, 30 aa, Artificial Sequence
  source: /note=“Description of Artificial Sequence Synthetic polypeptide”
YSEGTFTSDYRILKEEEAVRLFIEWLLAGG

SEQ ID NO: 234 - protein, 30 aa, Artificial Sequence
  source: /note=“Description of Artificial Sequence Synthetic polypeptide”
YSEGTFTSDYMMLKEEEAVRLFIEWLLAGG

SEQ ID NO: 235 - protein, 30 aa, Artificial Sequence
  source: /note=“Description of Artificial Sequence Synthetic polypeptide”
YSEGTFTSDYRMLKEEEAVRLFIEWLLAGG

SEQ ID NO: 236 - protein, 30 aa, Artificial Sequence
  source: /note=“Description of Artificial Sequence Synthetic polypeptide”
YSEGTFTSLYSKLKEEEAVRLFIEWLLAGG

SEQ ID NO: 237 - protein, 30 aa, Artificial Sequence
  source: /note=“Description of Artificial Sequence Synthetic polypeptide”
YSEGMHTSDLSKLLEEEAVRLFIEWLLAGG

SEQ ID NO: 238 - protein, 30 aa, Artificial Sequence
  source: /note=“Description of Artificial Sequence Synthetic polypeptide”
YSEGTFTSDYSIYLDEEAVRLFIEWLLAGG

SEQ ID NO: 239 - protein, 30 aa, Artificial Sequence
  source: /note=“Description of Artificial Sequence Synthetic polypeptide”
YSEGTFTSLYSKLLEEEAVRLFIEWLLAGG

SEQ ID NO: 240 - protein, 30 aa, Artificial Sequence
  source: /note=“Description of Artificial Sequence Synthetic polypeptide”
YSEGTHTSDFSKLLEEEAVRLFIEWLLAGG

SEQ ID NO: 241 - protein, 30 aa, Artificial Sequence
  source: /note=“Description of Artificial Sequence Synthetic polypeptide”
YSEGTFTSDYSKLLEEEAVRLFIEFLLAGG

SEQ ID NO: 242 - protein, 30 aa, Artificial Sequence
  source: /note=“Description of Artificial Sequence Synthetic polypeptide”
YSEGTFTSDYSKLLEEEAVRLFIEYLLAGG

SEQ ID NO: 243 - protein, 30 aa, Artificial Sequence
  source: /note=“Description of Artificial Sequence Synthetic polypeptide”
YSEGTFTSLYSKLLEEEAVRLFIEYLLAGG

SEQ ID NO: 244 - protein, 30 aa, Artificial Sequence
  source: /note=“Description of Artificial Sequence Synthetic polypeptide”
YSEGTFTSLYRILLEEEAVRLFIEWLLAGG

SEQ ID NO: 245 - protein, 30 aa, Artificial Sequence
  source: /note=“Description of Artificial Sequence Synthetic polypeptide”
YSEGTFTSDYSKLLEEEAVRLFIEWLLKGG

SEQ ID NO: 246 - protein, 30 aa, Artificial Sequence; identical to SEQ ID NO: 15
  source: /note=“Description of Artificial Sequence Synthetic polypeptide”

SEQ ID NO: 247 - protein, 30 aa, Artificial Sequence
  source: /note=“Description of Artificial Sequence Synthetic polypeptide”
YSEGTHTSDLSKLLEEEAVRLFIEWLLAGG

SEQ ID NO: 248 - protein, 30 aa, Artificial Sequence
  source: /note=“Description of Artificial Sequence Synthetic polypeptide”
YSEGTHTSDLRILLEEEAVRLFIEKLLAGG

SEQ ID NO: 249 - protein, 30 aa, Artificial Sequence
  source: /note=“Description of Artificial Sequence Synthetic polypeptide”
YSEGTFTSDYSKLLEEEAIRLFIEWLLAGG

SEQ ID NO: 250 - protein, 30 aa, Artificial Sequence
  source: /note=“Description of Artificial Sequence Synthetic polypeptide”
YSEGTFTSLYRIHKEEEAVRLFIEKLLAGG

SEQ ID NO: 251 - protein, 30 aa, Artificial Sequence
  source: /note=“Description of Artificial Sequence Synthetic polypeptide”
YGEGTFTSDYSKLLEEEAVRLFIEWLVKGP

SEQ ID NO: 252 - protein, 30 aa, Artificial Sequence
  source: /note=“Description of Artificial Sequence Synthetic polypeptide”
YSEGTFTSDYSKLLEEEANRLFIEWLLAGG

SEQ ID NO: 253 - protein, 30 aa, Artificial Sequence
  source: /note=“Description of Artificial Sequence Synthetic polypeptide”
YSEGTFTSDYSKLLEEEASRLFIEWLLAGG

SEQ ID NO: 254 - protein, 30 aa, Artificial Sequence
  source: /note=“Description of Artificial Sequence Synthetic polypeptide”
HSQGTFTSDYSKILEEEAVRLFIEWLLAGG

SEQ ID NO: 255 - protein, 30 aa, Artificial Sequence; identical to SEQ ID NO: 8
  source: /note=“Description of Artificial Sequence Synthetic polypeptide”

SEQ ID NO: 256 - protein, 30 aa, Artificial Sequence
  source: /note=“Description of Artificial Sequence Synthetic polypeptide”
YSEGTFTSDYSKLLEEEATRLFIEWLLAGG

SEQ ID NO: 257 - protein, 30 aa, Artificial Sequence
  source: /note=“Description of Artificial Sequence Synthetic polypeptide”
YSEGTFTSDVSSYLERQAIDEFIAWLLKGG

SEQ ID NO: 258 - protein, 30 aa, Artificial Sequence
  source: /note=“Description of Artificial Sequence Synthetic polypeptide”
YSEGTFTSDVSSYLEREAIDEFIAWLLKGG

SEQ ID NO: 259 - protein, 30 aa, Artificial Sequence
  source: /note=“Description of Artificial Sequence Synthetic polypeptide”
YSEGTFTSDVSSYLERQAIEEFIAWLLKGG

SEQ ID NO: 260 - protein, 30 aa, Artificial Sequence
  source: /note=“Description of Artificial Sequence Synthetic polypeptide”
YSEGTFTSDYSKLLERQAIDEFIAWLLKGG

SEQ ID NO: 261 - protein, 30 aa, Artificial Sequence
  source: /note=“Description of Artificial Sequence Synthetic polypeptide”
YSEGTFTSDVSSYMERQAIDEFIAWLLKQG

SEQ ID NO: 262 - protein, 30 aa, Artificial Sequence
  source: /note=“Description of Artificial Sequence Synthetic polypeptide”
YSEGTFISDVSSYLERQAIDEFIAWLLKGG

SEQ ID NO: 263 - protein, 30 aa, Artificial Sequence
  source: /note=“Description of Artificial Sequence Synthetic polypeptide”
YSEGTFTSDVSSYMERQAIDEFIAWLLKGK

SEQ ID NO: 264 - protein, 30 aa, Artificial Sequence
  source: /note=“Description of Artificial Sequence Synthetic polypeptide”
YSEGTFISDVSSYMERQAIDEFIAWLLKGG

SEQ ID NO: 265 - protein, 30 aa, Artificial Sequence
  source: /note=“Description of Artificial Sequence Synthetic polypeptide”
YSEGTFISDYSSYLERQAIDEFIAWLLKGG

SEQ ID NO: 266 - protein, 30 aa, Artificial Sequence
  source: /note=“Description of Artificial Sequence Synthetic polypeptide”
YSEGTFTSDVSSYLEKQAIDEFIAWLLKGG

SEQ ID NO: 267 - protein, 30 aa, Artificial Sequence
  source: /note=“Description of Artificial Sequence Synthetic polypeptide”
YSEGTFTSDYSKLLERQAIDEFIAELLKGG

SEQ ID NO: 268 - protein, 30 aa, Artificial Sequence
  source: /note=“Description of Artificial Sequence Synthetic polypeptide”
YSEGTFTSDYSKLLERQAIDEFIALLLKGG

SEQ ID NO: 269 - protein, 30 aa, Artificial Sequence
  source: /note=“Description of Artificial Sequence Synthetic polypeptide”
YSEGTFTSDYSKLLERQAIDEFIADLLKGG

SEQ ID NO: 270 - protein, 30 aa, Artificial Sequence
  source: /note=“Description of Artificial Sequence Synthetic polypeptide”
YSEGTFTSDYAAAAERQAIDAFAAWLAAAA

SEQ ID NO: 271 - protein, 30 aa, Artificial Sequence
  source: /note=“Description of Artificial Sequence Synthetic polypeptide”
YSEGTFTSDYSKLLERQAIDEFIAFLLKGG

SEQ ID NO: 272 - protein, 30 aa, Artificial Sequence
  source: /note=“Description of Artificial Sequence Synthetic polypeptide”
YSEGTFTSDYSKLLERQAIDEFIAILLKGG

SEQ ID NO: 273 - protein, 30 aa, Artificial Sequence
  source: /note=“Description of Artificial Sequence Synthetic polypeptide”
YSEGTFTSDYSKLLERQAIDEFIASLLKGG

SEQ ID NO: 274 - protein, 30 aa, Artificial Sequence
  source: /note=“Description of Artificial Sequence Synthetic polypeptide”
YSEGTFTSDYSKLLERQAIDEFIANLLKGG

SEQ ID NO: 275 - protein, 30 aa, Artificial Sequence
  source: /note=“Description of Artificial Sequence Synthetic polypeptide”
YSEGTFTSDYSKLLERQAIDEFIAGLLKGG

SEQ ID NO: 276 - protein, 30 aa, Artificial Sequence
  source: /note=“Description of Artificial Sequence Synthetic polypeptide”
YSEGTFTSDYSKLLERQAIDEFIAMLLKGE

SEQ ID NO: 277 - protein, 30 aa, Artificial Sequence
  source: /note=“Description of Artificial Sequence Synthetic polypeptide”
YSEGTFTSDFSKLLERQAIDEFIAWLLKGG

SEQ ID NO: 278 - protein, 30 aa, Artificial Sequence
  source: /note=“Description of Artificial Sequence Synthetic polypeptide”
YSEGMHTSDFSKLLERQAIDEFIAWLLKGG

SEQ ID NO: 279 - protein, 30 aa, Artificial Sequence
  source: /note=“Description of Artificial Sequence Synthetic polypeptide”
YSEGTFTSDYRMLKERQAIDEFIAWLLKGG

SEQ ID NO: 280 - protein, 30 aa, Artificial Sequence
  source: /note=“Description of Artificial Sequence Synthetic polypeptide”
YSEGTFTSDYRILKERQAIDEFIAWLLKGG

SEQ ID NO: 281 - protein, 30 aa, Artificial Sequence
  source: /note=“Description of Artificial Sequence Synthetic polypeptide”
YSEGTFTSDYRQAIDRQAIDEFIAWLLKGG

SEQ ID NO: 282 - protein, 30 aa, Artificial Sequence
  source: /note=“Description of Artificial Sequence Synthetic polypeptide”
YSEGTFTSDYSKLLERQAIDEFIAHLLKGG

SEQ ID NO: 283 - protein, 30 aa, Artificial Sequence
  source: /note=“Description of Artificial Sequence Synthetic polypeptide”
YSEGTHTSDFSKLLERQAIDEFIAWLLKGG

SEQ ID NO: 284 - protein, 30 aa, Artificial Sequence
  source: /note=“Description of Artificial Sequence Synthetic polypeptide”
YSEGTHTSLFSKLLERQAIDEFIAWLLKGG

SEQ ID NO: 285 - protein, 30 aa, Artificial Sequence
  source: /note=“Description of Artificial Sequence Synthetic polypeptide”
YSEGTFTSLFSKLLERQAIDEFIAWLLKGG

SEQ ID NO: 286 - protein, 30 aa, Artificial Sequence
  source: /note=“Description of Artificial Sequence Synthetic polypeptide”
YSEGTFTSLYRILLERQAIDEFIAWLLKGG

SEQ ID NO: 287 - protein, 30 aa, Artificial Sequence
  source: /note=“Description of Artificial Sequence Synthetic polypeptide”
YSEGTFTSDYSIYLDRQAIDEFIAWLLKGG

SEQ ID NO: 288 - protein, 30 aa, Artificial Sequence
  source: /note=“Description of Artificial Sequence Synthetic polypeptide”
YSEGMHTSDLSKLLERQAIDEFIAWLLKGG

SEQ ID NO: 289 - protein, 30 aa, Artificial Sequence; identical to SEQ ID NO: 282
  source: /note=“Description of Artificial Sequence Synthetic polypeptide”

SEQ ID NO: 290 - protein, 30 aa, Artificial Sequence; identical to SEQ ID NO: 260
  source: /note=“Description of Artificial Sequence Synthetic polypeptide”

SEQ ID NO: 291 - protein, 30 aa, Artificial Sequence
  source: /note=“Description of Artificial Sequence Synthetic polypeptide”
YSEGTHTSDLSKLLERQAIDEFIAWLLKGG

SEQ ID NO: 292 - protein, 30 aa, Artificial Sequence
  source: /note=“Description of Artificial Sequence Synthetic polypeptide”
YSEGTFTSLYRIHKERQAIDEFIAKLLKGG

SEQ ID NO: 293 - protein, 30 aa, Artificial Sequence
  source: /note=“Description of Artificial Sequence Synthetic polypeptide”
YSEGTHTSDLRILLERQAIDEFIAKLLKGG

SEQ ID NO: 294 - protein, 30 aa, Artificial Sequence
  source: /note=“Description of Artificial Sequence Synthetic polypeptide”
YSEGTHTSDLRILLERQAIDEFIAFLLKGG

SEQ ID NO: 295 - protein, 30 aa, Artificial Sequence
  source: /note=“Description of Artificial Sequence Synthetic polypeptide”
YSEGTFISDYSIALAAAAVRDFVNWLLAGG

SEQ ID NO: 296 - protein, 30 aa, Artificial Sequence
  source: /note=“Description of Artificial Sequence Synthetic polypeptide”
YSEGTFISDYSKLLAAAAVRDFVNWLLAGG

SEQ ID NO: 297 - protein, 30 aa, Artificial Sequence
  source: /note=“Description of Artificial Sequence Synthetic polypeptide”
YSEGTFISDYSIAMDEEAVRDFVEWLKAGG

SEQ ID NO: 298 - protein, 39 aa, Artificial Sequence; identical to SEQ ID NO: 62
  source: /note=“Description of Artificial Sequence Synthetic polypeptide”

SEQ ID NO: 299 - protein, 40 aa, Artificial Sequence
  source: /note=“Description of Artificial Sequence Synthetic polypeptide”
HGEGTFTSDLSKQMEEECVRLFIEWLKNGGPSSGAPPPGC

SEQ ID NO: 300 - protein, 30 aa, Artificial Sequence
  source: /note=“Description of Artificial Sequence Synthetic polypeptide”
YSEGTHTSDLRILLEEEAVRLFIEFLLAGG

SEQ ID NO: 301 - protein, 30 aa, Artificial Sequence
  source: /note=“Description of Artificial Sequence Synthetic polypeptide”
YSEGTFISDYSIALAAAAQQDFVNWLLAGG

SEQ ID NO: 302 - protein, 30 aa, Artificial Sequence
  source: /note=“Description of Artificial Sequence Synthetic polypeptide”
YSEGTFISDYSIALAAAAAKDFVNWLLAGG

SEQ ID NO: 303 - protein, 30 aa, Artificial Sequence
  source: /note=“Description of Artificial Sequence Synthetic polypeptide”
YSEGTFISDYSIALAAAAIDDFVNWLLAGG

SEQ ID NO: 304 - protein, 30 aa, Artificial Sequence
  source: /note=“Description of Artificial Sequence Synthetic polypeptide”
YSEGTFTSDYSKLLEAAAAAAFAAWLAAAA

SEQ ID NO: 305 - protein, 30 aa, Artificial Sequence
  source: /note=“Description of Artificial Sequence Synthetic polypeptide”
YSEGTFTSDYAAAAEAAAAAAFAAWLAAAA

SEQ ID NO: 306 - protein, 30 aa, Artificial Sequence
  source: /note=“Description of Artificial Sequence Synthetic polypeptide”
YSEGTFTSDYSIALAAAAQQDFVNWLLAGG

SEQ ID NO: 307 - protein, 30 aa, Artificial Sequence
  source: /note=“Description of Artificial Sequence Synthetic polypeptide”
YSEGTFISDYSIALAAAAQQDFVNWLLKGG

SEQ ID NO: 308 - protein, 30 aa, Artificial Sequence
  source: /note=“Description of Artificial Sequence Synthetic polypeptide”
YSEGTFTSDYSIALAAAAQQDFVNWLLKGG

SEQ ID NO: 309 - protein, 30 aa, Artificial Sequence
  source: /note=“Description of Artificial Sequence Synthetic polypeptide”
YSEGTFTSDYSAAAEAAAAAAFAAWLAAAA

SEQ ID NO: 310 - protein, 30 aa, Artificial Sequence
  source: /note=“Description of Artificial Sequence Synthetic polypeptide”
YSEGTFTSDYAAAAEAAAAAAFAAWLAKAA

SEQ ID NO: 311 - protein, 30 aa, Artificial Sequence
  source: /note=“Description of Artificial Sequence Synthetic polypeptide”
YSEGTFISDYSIAMDKIHQQDFVNWLLAGG

SEQ ID NO: 312 - protein, 30 aa, Artificial Sequence; identical to SEQ ID NO: 63
  source: /note=“Description of Artificial Sequence Synthetic polypeptide”

SEQ ID NO: 313 - protein, 30 aa, Artificial Sequence; identical to SEQ ID NO: 63
  source: /note=“Description of Artificial Sequence Synthetic polypeptide”

SEQ ID NO: 314 - protein, 30 aa, Artificial Sequence; identical to SEQ ID NO: 63
  source: /note=“Description of Artificial Sequence Synthetic polypeptide”

SEQ ID NO: 315 - protein, 30 aa, Artificial Sequence; identical to SEQ ID NO: 63
  source: /note=“Description of Artificial Sequence Synthetic polypeptide”

SEQ ID NO: 316 - protein, 30 aa, Artificial Sequence; identical to SEQ ID NO: 63
  source: /note=“Description of Artificial Sequence Synthetic polypeptide”

SEQ ID NO: 317 - protein, 39 aa, Artificial Sequence
  source: /note=“Description of Artificial Sequence Synthetic polypeptide”
YSEGTFISDYSIAMDKIHQQDFVNWLLAQKPSSGAPPPS

SEQ ID NO: 318 - protein, 30 aa, Artificial Sequence; identical to SEQ ID NO: 63
  source: /note=“Description of Artificial Sequence Synthetic polypeptide”

SEQ ID NO: 319 - protein, 30 aa, Artificial Sequence; identical to SEQ ID NO: 63
  source: /note=“Description of Artificial Sequence Synthetic polypeptide”

SEQ ID NO: 320 - protein, 30 aa, Artificial Sequence; identical to SEQ ID NO: 63
  source: /note=“Description of Artificial Sequence Synthetic polypeptide”

SEQ ID NO: 321 - protein, 30 aa, Artificial Sequence; identical to SEQ ID NO: 63
  source: /note=“Description of Artificial Sequence Synthetic polypeptide”

SEQ ID NO: 322 - protein, 42 aa, Artificial Sequence
  source: /note=“Description of Artificial Sequence Synthetic polypeptide”
YSEGTFISDYSIAMDKIHQQDFVNWLLAQKGAANDWKHNITQ

SEQ ID NO: 323 - protein, 42 aa, Artificial Sequence
  source: /note=“Description of Artificial Sequence Synthetic polypeptide”
YSEGTFISDYSIAMDKIHQQDFVNWLLAQKGKKNDWKHNITQ

SEQ ID NO: 324 - protein, 30 aa, Artificial Sequence; identical to SEQ ID NO: 63
  source: /note=“Description of Artificial Sequence Synthetic polypeptide”

SEQ ID NO: 325 - protein, 30 aa, Artificial Sequence; identical to SEQ ID NO: 63
  source: /note=“Description of Artificial Sequence Synthetic polypeptide”

SEQ ID NO: 326 - protein, 63 aa, Artificial Sequence; identical to SEQ ID NO: 91
  source: /note=“Description of Artificial Sequence Synthetic polypeptide”

SEQ ID NO: 327 - protein, 59 aa, Artificial Sequence
  source: /note=“Description of Artificial Sequence Synthetic polypeptide”
YSEGTFTSDYSKLLEEEHVRLFIEWLVKGPGGGGGSGGGGSGGGGSGGGGSATHTCPPC

SEQ ID NO: 328 - protein, 59 aa, Artificial Sequence
  source: /note=“Description of Artificial Sequence Synthetic polypeptide”
YSEGTFTSDYSKLLEEEAVRLFIEWLVKGPGGGGGSGGGGSGGGGSGGGGSATHTCPPC

SEQ ID NO: 329 - protein, 61 aa, Artificial Sequence
  source: /note=“Description of Artificial Sequence Synthetic polypeptide”
YSEGTFTSDYSKLLEEEAVRLFIEWLLAQKAPPGGSGSTASSGSGSATGGGGAATHTCPPC

SEQ ID NO: 330 - protein, 61 aa, Artificial Sequence
  source: /note=“Description of Artificial Sequence Synthetic polypeptide”
YSEGTFTSDYSKLLEEEAVRLFIEWLVKGPAAAGGSGSTASSGSGSATGGGGAATHTCPPC

SEQ ID NO: 331 - protein, 59 aa, Artificial Sequence
  source: /note=“Description of Artificial Sequence Synthetic polypeptide”
YSEGTFTSDYSKLLEKEAVRLFIEWLLKGPGGGGGSGGGGSGGGGSGGGGSATHTCPPC

SEQ ID NO: 332 - protein, 59 aa, Artificial Sequence
  source: /note=“Description of Artificial Sequence Synthetic polypeptide”
YSEGTFTSDYSKLLEKEAQRLFIEWLLKGPGGGGGSGGGGSGGGGSGGGGSATHTCPPC

SEQ ID NO: 333 - protein, 55 aa, Artificial Sequence
  source: /note=“Description of Artificial Sequence Synthetic polypeptide”
YSEGTFTSDYSKLLEEEAVRLFIEWLVKGPGGGGGGGGGGGGGGGGGATHTCPPC

SEQ ID NO: 334 - protein, 68 aa, Artificial Sequence
  source: /note=“Description of Artificial Sequence Synthetic polypeptide”
YSEGTFTSDYSKLLEEEAVRLFIEWLVKGPPSSGAPPPSGGGGGSGGGGSGGGGSGGGGSATHTCPPC

SEQ ID NO: 335 - protein, 67 aa, Artificial Sequence
  source: /note=“Description of Artificial Sequence Synthetic polypeptide”
YSEGTFTSDYSKLLEEEAVRLFIEWLVKGPSSGAPPPSGGGGGSGGGGSGGGGSGGGGSATHTCPPC

SEQ ID NO: 336 - protein, 68 aa, Artificial Sequence
  source: /note=“Description of Artificial Sequence Synthetic polypeptide”
YSEGTFTSDYSKLLEEEAVRLFIEWLVKGGPSSGAPPPSGGGGGSGGGGSGGGGSGGGGSATHTCPPC

SEQ ID NO: 337 - protein, 56 aa, Artificial Sequence
  source: /note=“Description of Artificial Sequence Synthetic polypeptide”
YSEGTFTSDYSKLLEEEAVRLFIEWLVKGPGGGGGSGGGGSGGGGSGGGGSACPPC

SEQ ID NO: 338 - protein, 45 aa, Artificial Sequence
  source: /note=“Description of Artificial Sequence Synthetic polypeptide”
YSEGTFTSDYSKLLEEEAVRLFIEWLVKGPGGGGGGGATHTCPPC

SEQ ID NO: 339 - protein, 50 aa, Artificial Sequence
  source: /note=“Description of Artificial Sequence Synthetic polypeptide”
YSEGTFTSDYSKLLEEEAVRLFIEWLVKGPGGGGGGGGGGGGATHTCPPC

SEQ ID NO: 340 - protein, 61 aa, Artificial Sequence
  source: /note=“Description of Artificial Sequence Synthetic polypeptide”
YSEGTFTSDYSKLLEEEAVRLFIEWLVKGPGGGGGGGGGGGGGGGGGGGGGGGATHTCPPC

SEQ ID NO: 341 - protein, 65 aa, Artificial Sequence
  source: /note=“Description of Artificial Sequence Synthetic polypeptide”
YSEGTFTSDYSKLLEEEAVRLFIEWLVKGPGGGGGGGGGGGGGGGGGGGGGGGGGGGATHTCPPC

SEQ ID NO: 342 - protein, 59 aa, Artificial Sequence
  source: /note=“Description of Artificial Sequence Synthetic polypeptide”
YSEGTFTSDYSKLLEKEAERLFIEWLLKGPGGGGGSGGGGSGGGGSGGGGSATHTCPPC

SEQ ID NO: 343 - protein, 55 aa, Artificial Sequence
  source: /note=“Description of Artificial Sequence Synthetic polypeptide”
YSEGTFTSDYSKLLEEEAVRLFIEWLVKGPGGGGGGGGGGGGGGGGGGGGACPPC

SEQ ID NO: 344 - protein, 62 aa, Artificial Sequence
  source: /note=“Description of Artificial Sequence Synthetic polypeptide”
YSEGTFTSDYSKLLEEECVRLFIEWLVKGGPGGGAPPPGCGGGGGGGGGGGGGGGGGACPPC

SEQ ID NO: 345 - protein, 62 aa, Artificial Sequence
  source: /note=“Description of Artificial Sequence Synthetic polypeptide”
YSEGTFTSDYSKLLEEEAVRCFIEWLVKGGPGGGAPPCGGGGGGGGGGGGGGGGGGGACPPC

SEQ ID NO: 346 - protein, 55 aa, Artificial Sequence
  source: /note=“Description of Artificial Sequence Synthetic polypeptide”
YSEGTFTSDYSKLLEEEAVRCFIECLVKGPGGGGGGGGGGGGGGGGGGGGACPPC

SEQ ID NO: 347 - protein, 55 aa, Artificial Sequence
  source: /note=“Description of Artificial Sequence Synthetic polypeptide”
YSEGTFTSDYSKLLEEEAVRLFICWLVCGPGGGGGGGGGGGGGGGGGGGGACPPC

SEQ ID NO: 348 - protein, 68 aa, Artificial Sequence
  source: /note=“Description of Artificial Sequence Synthetic polypeptide”
YSEGTFTSDYSKLLEEEAVRLFIEWLLAGGPSSGAPPPSGGGGGSGGGGSGGGGSGGGGSATHTCPPC

SEQ ID NO: 349 - protein, 68 aa, Artificial Sequence
  source: /note=“Description of Artificial Sequence Synthetic polypeptide”
YSEGTFTSDYSKLLEEEAVRCFIEWLVKGGPSSGAPPCSGGGGGGGGGGGGGGGGGGGGGGGGACPPC

SEQ ID NO: 350 - protein, 58 aa, Artificial Sequence
  source: /note=“Description of Artificial Sequence Synthetic polypeptide”
YSEGTFTSDYSKLLEEEAVRLFIEWLLAGGGGGGGGGGGGGGGGGGGGGGGGGACPPC

SEQ ID NO: 351 - protein, 59 aa, Artificial Sequence
  source: /note=“Description of Artificial Sequence Synthetic polypeptide”
YSEGTFISDYSKLLEEEAVRDFVEWLKAGGGGGGGSGGGGSGGGGSGGGGSATHTCPPC

SEQ ID NO: 352 - protein, 58 aa, Artificial Sequence
  source: /note=“Description of Artificial Sequence Synthetic polypeptide”
YSEGTFTSDYSKLLEEEAVRLFIEWLLAGAPPGGGGGGGGGGGGGGGGGGGGGACPPC

SEQ ID NO: 353 - protein, 57 aa, Artificial Sequence
  source: /note=“Description of Artificial Sequence Synthetic polypeptide”
YSEGTFTSDYSKLLEEEAVRLFIEWLVKGPGGGGGSGGGGSGGGGSGGGGSGGGGSA

SEQ ID NO: 354 - protein, 62 aa, Artificial Sequence
  source: /note=“Description of Artificial Sequence Synthetic polypeptide”
YSEGTFTSDYSKLLEEEAVRLFIEWLVKGPGGGGGSGGGGSGGGGSGGGGSGGGGSGGGGSA

SEQ ID NO: 355 - protein, 67 aa, Artificial Sequence
  source: /note=“Description of Artificial Sequence Synthetic polypeptide”
YSEGTFTSDYSKLLEEEAVRLFIEWLVKGPGGGGGSGGGGSGGGGSGGGGSGGGGSGGGGSGGGGSA

SEQ ID NO: 356 - protein, 72 aa, Artificial Sequence
  source: /note=“Description of Artificial Sequence Synthetic polypeptide”
YSEGTFTSDYSKLLEEEAVRLFIEWLVKGPGGGGGSGGGGSGGGGSGGGGSGGGGSGGGGSGGGGSGGGGSA

SEQ ID NO: 357 - protein, 57 aa, Artificial Sequence
  source: /note=“Description of Artificial Sequence Synthetic polypeptide”
YSEGTFTSDYSKLLEEEAVRLFIEWLLAGGGGGGGSGGGGSGGGGSGGGGSGGGGSA

SEQ ID NO: 358 - protein, 62 aa, Artificial Sequence
  source: /note=“Description of Artificial Sequence Synthetic polypeptide”
YSEGTFTSDYSKLLEEEAVRLFIEWLLAGGGGGGGSGGGGSGGGGSGGGGSGGGGSGGGGSA

SEQ ID NO: 359 - protein, 72 aa, Artificial Sequence
  source: /note=“Description of Artificial Sequence Synthetic polypeptide”
YSEGTFTSDYSKLLEEEAVRLFIEWLLAGGGGGGGSGGGGSGGGGSGGGGSGGGGSGGGGSGGGGSGGGGSA

SEQ ID NO: 360 - protein, 62 aa, Artificial Sequence
  source: /note=“Description of Artificial Sequence Synthetic polypeptide”
YSEGTFTSDYSKLLEKEAQRLFIEWLLAGGGGGGGSGGGGSGGGGSGGGGSGGGGSGGGGSA

SEQ ID NO: 361 - protein, 67 aa, Artificial Sequence
  source: /note=“Description of Artificial Sequence Synthetic polypeptide”
HGEGTFTSDLSKQMEEEAVRLFIEWLKNGGPSSGAPPPSGSGGGGSGGGGSGGGGSGGGGSGGGGSA

SEQ ID NO: 362 - protein, 58 aa, Artificial Sequence
  source: /note=“Description of Artificial Sequence Synthetic polypeptide”
YSEGMHTSDFSKLLEEEAVRLFIEWLLAGGGGGGSGGGGSGGGGSGGGGSATHTCPPC

SEQ ID NO: 363 - protein, 58 aa, Artificial Sequence
  source: /note=“Description of Artificial Sequence Synthetic polypeptide”
YSEGTHTSLFSKLLEEEAVRLFIEWLLAGGGGGGSGGGGSGGGGSGGGGSATHTCPPC

SEQ ID NO: 364 - protein, 58 aa, Artificial Sequence
  source: /note=“Description of Artificial Sequence Synthetic polypeptide”
YSEGTFTSDFSKLLEEEAVRLFIEFLLAGGGGGGSGGGGSGGGGSGGGGSATHTCPPC

SEQ ID NO: 365 - protein, 58 aa, Artificial Sequence
  source: /note=“Description of Artificial Sequence Synthetic polypeptide”
YSEGTFTSDFSKLLEEEAVRLFIEWLLAGGGGGGSGGGGSGGGGSGGGGSATHTCPPC

SEQ ID NO: 366 - protein, 58 aa, Artificial Sequence
  source: /note=“Description of Artificial Sequence Synthetic polypeptide”
YSEGTFTSDYSKLLEEEAVRLFIEHLLAGGGGGGSGGGGSGGGGSGGGGSATHTCPPC

SEQ ID NO: 367 - protein, 58 aa, Artificial Sequence
  source: /note=“Description of Artificial Sequence Synthetic polypeptide”
YSEGTFTSDYRILLEEEAVRLFIEWLLAGGGGGGSGGGGSGGGGSGGGGSATHTCPPC

SEQ ID NO: 368 - protein, 58 aa, Artificial Sequence
  source: /note=“Description of Artificial Sequence Synthetic polypeptide”
YSEGTFTSDYRILKEEEAVRLFIEWLLAGGGGGGSGGGGSGGGGSGGGGSATHTCPPC

SEQ ID NO: 369 - protein, 58 aa, Artificial Sequence
  source: /note=“Description of Artificial Sequence Synthetic polypeptide”
YSEGTFTSDYMMLKEEEAVRLFIEWLLAGGGGGGSGGGGSGGGGSGGGGSATHTCPPC

SEQ ID NO: 370 - protein, 58 aa, Artificial Sequence
  source: /note=“Description of Artificial Sequence Synthetic polypeptide”
YSEGTFTSDYRMLKEEEAVRLFIEWLLAGGGGGGSGGGGSGGGGSGGGGSATHTCPPC

SEQ ID NO: 371 - protein, 58 aa, Artificial Sequence
  source: /note=“Description of Artificial Sequence Synthetic polypeptide”
YSEGTFTSLYSKLKEEEAVRLFIEWLLAGGGGGGSGGGGSGGGGSGGGGSATHTCPPC

SEQ ID NO: 372 - protein, 56 aa, Artificial Sequence
  source: /note=“Description of Artificial Sequence Synthetic polypeptide”
YSEGMHTSDLSKLLEEEAVRLFIEWLLAGGGGGGGSGGGGSGGGGSGGGGSACPPC

SEQ ID NO: 373 - protein, 56 aa, Artificial Sequence
  source: /note=“Description of Artificial Sequence Synthetic polypeptide”
YSEGTFTSDYSIYLDEEAVRLFIEWLLAGGGGGGGSGGGGSGGGGSGGGGSACPPC

SEQ ID NO: 374 - protein, 58 aa, Artificial Sequence
  source: /note=“Description of Artificial Sequence Synthetic polypeptide”
YSEGTFTSLYSKLLEEEAVRLFIEWLLAGGGGGGSGGGGSGGGGSGGGGSATHTCPPC

SEQ ID NO: 375 - protein, 58 aa, Artificial Sequence
  source: /note=“Description of Artificial Sequence Synthetic polypeptide”
YSEGTHTSDFSKLLEEEAVRLFIEWLLAGGGGGGSGGGGSGGGGSGGGGSATHTCPPC

SEQ ID NO: 376 - protein, 58 aa, Artificial Sequence
  source: /note=“Description of Artificial Sequence Synthetic polypeptide”
YSEGTFTSDYSKLLEEEAVRLFIEFLLAGGGGGGSGGGGSGGGGSGGGGSATHTCPPC

SEQ ID NO: 377 - protein, 58 aa, Artificial Sequence
  source: /note=“Description of Artificial Sequence Synthetic polypeptide”
YSEGTFTSDYSKLLEEEAVRLFIEYLLAGGGGGGSGGGGSGGGGSGGGGSATHTCPPC

SEQ ID NO: 378 - protein, 58 aa, Artificial Sequence
  source: /note=“Description of Artificial Sequence Synthetic polypeptide”
YSEGTFTSLYSKLLEEEAVRLFIEYLLAGGGGGGSGGGGSGGGGSGGGGSATHTCPPC

SEQ ID NO: 379 - protein, 58 aa, Artificial Sequence
  source: /note=“Description of Artificial Sequence Synthetic polypeptide”
YSEGTFTSLYRILLEEEAVRLFIEWLLAGGGGGGSGGGGSGGGGSGGGGSATHTCPPC

SEQ ID NO: 380 - protein, 58 aa, Artificial Sequence
  source: /note=“Description of Artificial Sequence Synthetic polypeptide”
YSEGTFTSDYSKLLEEEAVRLFIEWLLKGGGGGGSGGGGSGGGGSGGGGSATHTCPPC

SEQ ID NO: 381 - protein, 61 aa, Artificial Sequence
  source: /note=“Description of Artificial Sequence Synthetic polypeptide”
YSEGTFTSDYSKLLEEEAVRLFIEWLVKGPGGGGGSGGGGSGGGGSGGGGSGGGGSACPPC

SEQ ID NO: 382 - protein, 58 aa, Artificial Sequence
  source: /note=“Description of Artificial Sequence Synthetic polypeptide”
YSEGTHTSDLSKLLEEEAVRLFIEWLLAGGGGGGSGGGGSGGGGSGGGGSATHTCPPC

SEQ ID NO: 383 - protein, 72 aa, Artificial Sequence
  source: /note=“Description of Artificial Sequence Synthetic polypeptide”
YSEGTHTSDLRILLEEEAVRLFIEKLLAGGGSGGGGSGGGGSGGGGSGGGGSGGGGSGGGGSGGGGSACPPC

SEQ ID NO: 384 - protein, 58 aa, Artificial Sequence
  source: /note=“Description of Artificial Sequence Synthetic polypeptide”
YSEGTFTSDYSKLLEEEAIRLFIEWLLAGGGGGGSGGGGSGGGGSGGGGSATHTCPPC

SEQ ID NO: 385 - protein, 72 aa, Artificial Sequence
  source: /note=“Description of Artificial Sequence Synthetic polypeptide”
YSEGTFTSLYRIHKEEEAVRLFIEKLLAGGGSGGGGSGGGGSGGGGSGGGGSGGGGSGGGGSGGGGSACPPC

SEQ ID NO: 386 - protein, 55 aa, Artificial Sequence
  source: /note=“Description of Artificial Sequence Synthetic polypeptide”
YGEGTFTSDYSKLLEEEAVRLFIEWLVKGPGGGGGGGGGGGGGGGGGGGGACPPC

SEQ ID NO: 387 - protein, 58 aa, Artificial Sequence
  source: /note=“Description of Artificial Sequence Synthetic polypeptide”
YSEGTFTSDYSKLLEEEANRLFIEWLLAGGGGGGSGGGGSGGGGSGGGGSATHTCPPC

SEQ ID NO: 388 - protein, 58 aa, Artificial Sequence
  source: /note=“Description of Artificial Sequence Synthetic polypeptide”
YSEGTFTSDYSKLLEEEASRLFIEWLLAGGGGGGSGGGGSGGGGSGGGGSATHTCPPC

SEQ ID NO: 389 - protein, 72 aa, Artificial Sequence
  source: /note=“Description of Artificial Sequence Synthetic polypeptide”
HSQGTFTSDYSKILEEEAVRLFIEWLLAGGGSGGGGSGGGGSGGGGSGGGGSGGGGSGGGGSGGGGSACPPC

SEQ ID NO: 390 - protein, 72 aa, Artificial Sequence
  source: /note=“Description of Artificial Sequence Synthetic polypeptide”
YSEGTFTSDYSKLLEEEAVRLFIEWLLAGGGSGGGGSGGGGSGGGGSGGGGSGGGGSGGGGSGGGGSACPPC

SEQ ID NO: 391 - protein, 58 aa, Artificial Sequence
  source: /note=“Description of Artificial Sequence Synthetic polypeptide”
YSEGTFTSDYSKLLEEEATRLFIEWLLAGGGGGGSGGGGSGGGGSGGGGSATHTCPPC

SEQ ID NO: 392 - protein, 61 aa, Artificial Sequence
  source: /note=“Description of Artificial Sequence Synthetic polypeptide”
YSEGTFTSDVSSYLERQAIDEFIAWLLKGGAAAGGSGSTASSGSGSATGGGGAATHTCPPC

SEQ ID NO: 393 - protein, 61 aa, Artificial Sequence
  source: /note=“Description of Artificial Sequence Synthetic polypeptide”
YSEGTFTSDVSSYLEREAIDEFIAWLLKGGAAAGGSGSTASSGSGSATGGGGAATHTCPPC

SEQ ID NO: 394 - protein, 61 aa, Artificial Sequence
  source: /note=“Description of Artificial Sequence Synthetic polypeptide”
YSEGTFTSDVSSYLERQAIEEFIAWLLKGGAAAGGSGSTASSGSGSATGGGGAATHTCPPC

SEQ ID NO: 395 - protein, 61 aa, Artificial Sequence
  source: /note=“Description of Artificial Sequence Synthetic polypeptide”
YSEGTFTSDYSKLLERQAIDEFIAWLLKGGAAAGGSGSTASSGSGSATGGGGAATHTCPPC

SEQ ID NO: 396 - protein, 61 aa, Artificial Sequence
  source: /note=“Description of Artificial Sequence Synthetic polypeptide”
YSEGTFTSDVSSYMERQAIDEFIAWLLKQGAAAGGSGSTASSGSGSATGGGGAATHTCPPC

SEQ ID NO: 397 - protein, 61 aa, Artificial Sequence
  source: /note=“Description of Artificial Sequence Synthetic polypeptide”
YSEGTFISDVSSYLERQAIDEFIAWLLKGGAAAGGSGSTASSGSGSATGGGGAATHTCPPC

SEQ ID NO: 398 - protein, 61 aa, Artificial Sequence
  source: /note=“Description of Artificial Sequence Synthetic polypeptide”
YSEGTFTSDVSSYMERQAIDEFIAWLLKGKAAAGGSGSTASSGSGSATGGGGAATHTCPPC

SEQ ID NO: 399 - protein, 61 aa, Artificial Sequence
  source: /note=“Description of Artificial Sequence Synthetic polypeptide”
YSEGTFISDVSSYMERQAIDEFIAWLLKGGAAAGGSGSTASSGSGSATGGGGAATHTCPPC

SEQ ID NO: 400 - protein, 61 aa, Artificial Sequence
  source: /note=“Description of Artificial Sequence Synthetic polypeptide”
YSEGTFISDYSSYLERQAIDEFIAWLLKGGAAAGGSGSTASSGSGSATGGGGAATHTCPPC

SEQ ID NO: 401 - protein, 61 aa, Artificial Sequence
  source: /note=“Description of Artificial Sequence Synthetic polypeptide”
YSEGTFTSDVSSYLEKQAIDEFIAWLLKGGAAAGGSGSTASSGSGSATGGGGAATHTCPPC

SEQ ID NO: 402 - protein, 62 aa, Artificial Sequence
  source: /note=“Description of Artificial Sequence Synthetic polypeptide”
YSEGTFTSDYSKLLERQAIDEFIAELLKGGGGGGGSGGGGSGGGGSGGGGSGGGGSGGGGSA

SEQ ID NO: 403 - protein, 62 aa, Artificial Sequence
  source: /note=“Description of Artificial Sequence Synthetic polypeptide”
YSEGTFTSDYSKLLERQAIDEFIALLLKGGGGGGGSGGGGSGGGGSGGGGSGGGGSGGGGSA

SEQ ID NO: 404 - protein, 62 aa, Artificial Sequence
  source: /note=“Description of Artificial Sequence Synthetic polypeptide”
YSEGTFTSDYSKLLERQAIDEFIADLLKGGGGGGGSGGGGSGGGGSGGGGSGGGGSGGGGSA

SEQ ID NO: 405 - protein, 52 aa, Artificial Sequence
  source: /note=“Description of Artificial Sequence Synthetic polypeptide”
YSEGTFTSDYAAAAERQAIDAFAAWLAAAAGGGGGSGGGGSGGGGSGGGGSA

SEQ ID NO: 406 - protein, 62 aa, Artificial Sequence
  source: /note=“Description of Artificial Sequence Synthetic polypeptide”
YSEGTFTSDYSKLLERQAIDEFIAFLLKGGGGGGGSGGGGSGGGGSGGGGSGGGGSGGGGSA

SEQ ID NO: 407 - protein, 62 aa, Artificial Sequence
  source: /note=“Description of Artificial Sequence Synthetic polypeptide”
YSEGTFTSDYSKLLERQAIDEFIAILLKGGGGGGGSGGGGSGGGGSGGGGSGGGGSGGGGSA

SEQ ID NO: 408 - protein, 62 aa, Artificial Sequence
  source: /note=“Description of Artificial Sequence Synthetic polypeptide”
YSEGTFTSDYSKLLERQAIDEFIASLLKGGGGGGGSGGGGSGGGGSGGGGSGGGGSGGGGSA

SEQ ID NO: 409 - protein, 62 aa, Artificial Sequence
  source: /note=“Description of Artificial Sequence Synthetic polypeptide”
YSEGTFTSDYSKLLERQAIDEFIANLLKGGGGGGGSGGGGSGGGGSGGGGSGGGGSGGGGSA

SEQ ID NO: 410 - protein, 62 aa, Artificial Sequence
  source: /note=“Description of Artificial Sequence Synthetic polypeptide”
YSEGTFTSDYSKLLERQAIDEFIAGLLKGGGGGGGSGGGGSGGGGSGGGGSGGGGSGGGGSA

SEQ ID NO: 411 - protein, 62 aa, Artificial Sequence
  source: /note=“Description of Artificial Sequence Synthetic polypeptide”
YSEGTFTSDYSKLLERQAIDEFIAMLLKGEGGGGGSGGGGSGGGGSGGGGSGGGGSGGGGSA

SEQ ID NO: 412 - protein, 61 aa, Artificial Sequence
  source: /note=“Description of Artificial Sequence Synthetic polypeptide”
YSEGTFTSDFSKLLERQAIDEFIAWLLKGGAAAGGSGSTASSGSGSATGGGGAATHTCPPC

SEQ ID NO: 413 - protein, 61 aa, Artificial Sequence
  source: /note=“Description of Artificial Sequence Synthetic polypeptide”
YSEGMHTSDFSKLLERQAIDEFIAWLLKGGAAAGGSGSTASSGSGSATGGGGAATHTCPPC

SEQ ID NO: 414 - protein, 61 aa, Artificial Sequence
  source: /note=“Description of Artificial Sequence Synthetic polypeptide”
YSEGTFTSDYRMLKERQAIDEFIAWLLKGGAAAGGSGSTASSGSGSATGGGGAATHTCPPC

SEQ ID NO: 415 - protein, 61 aa, Artificial Sequence
  source: /note=“Description of Artificial Sequence Synthetic polypeptide”
YSEGTFTSDYRILKERQAIDEFIAWLLKGGAAAGGSGSTASSGSGSATGGGGAATHTCPPC

SEQ ID NO: 416 - protein, 56 aa, Artificial Sequence
  source: /note=“Description of Artificial Sequence Synthetic polypeptide”
YSEGTFTSDYRQAIDRQAIDEFIAWLLKGGGGGGGSGGGGSGGGGSGGGGSACPPC

SEQ ID NO: 417 - protein, 62 aa, Artificial Sequence
  source: /note=“Description of Artificial Sequence Synthetic polypeptide”
YSEGTFTSDYSKLLERQAIDEFIAHLLKGGGGGGGSGGGGSGGGGSGGGGSGGGGSGGGGSA

SEQ ID NO: 418 - protein, 61 aa, Artificial Sequence
  source: /note=“Description of Artificial Sequence Synthetic polypeptide”
YSEGTHTSDFSKLLERQAIDEFIAWLLKGGAAAGGSGSTASSGSGSATGGGGAATHTCPPC

SEQ ID NO: 419 - protein, 61 aa, Artificial Sequence
  source: /note=“Description of Artificial Sequence Synthetic polypeptide”
YSEGTHTSLFSKLLERQAIDEFIAWLLKGGAAAGGSGSTASSGSGSATGGGGAATHTCPPC

SEQ ID NO: 420 - protein, 61 aa, Artificial Sequence
  source: /note=“Description of Artificial Sequence Synthetic polypeptide”
YSEGTFTSLFSKLLERQAIDEFIAWLLKGGAAAGGSGSTASSGSGSATGGGGAATHTCPPC

SEQ ID NO: 421 - protein, 61 aa, Artificial Sequence
  source: /note=“Description of Artificial Sequence Synthetic polypeptide”
YSEGTFTSLYRILLERQAIDEFIAWLLKGGAAAGGSGSTASSGSGSATGGGGAATHTCPPC

SEQ ID NO: 422 - protein, 56 aa, Artificial Sequence
  source: /note=“Description of Artificial Sequence Synthetic polypeptide”
YSEGTFTSDYSIYLDRQAIDEFIAWLLKGGGGGGGSGGGGSGGGGSGGGGSACPPC

SEQ ID NO: 423 - protein, 61 aa, Artificial Sequence
  source: /note=“Description of Artificial Sequence Synthetic polypeptide”
YSEGMHTSDLSKLLERQAIDEFIAWLLKGGAAAGGSGSTASSGSGSATGGGGAATHTCPPC

SEQ ID NO: 424 - protein, 56 aa, Artificial Sequence
  source: /note=“Description of Artificial Sequence Synthetic polypeptide”
YSEGTFTSDYSKLLERQAIDEFIAHLLKGGGGGGGSGGGGSGGGGSGGGGSACPPC

SEQ ID NO: 425 - protein, 56 aa, Artificial Sequence
  source: /note=“Description of Artificial Sequence Synthetic polypeptide”
YSEGTFTSDYSKLLERQAIDEFIAWLLKGGGGGGGSGGGGSGGGGSGGGGSACPPC

SEQ ID NO: 426 - protein, 61 aa, Artificial Sequence
  source: /note=“Description of Artificial Sequence Synthetic polypeptide”
YSEGTHTSDLSKLLERQAIDEFIAWLLKGGAAAGGSGSTASSGSGSATGGGGAATHTCPPC

SEQ ID NO: 427 - protein, 72 aa, Artificial Sequence
  source: /note=“Description of Artificial Sequence Synthetic polypeptide”
YSEGTFTSLYRIHKERQAIDEFIAKLLKGGGSGGGGSGGGGSGGGGSGGGGSGGGGSGGGGSGGGGSACPPC

SEQ ID NO: 428 - protein, 72 aa, Artificial Sequence
  source: /note=“Description of Artificial Sequence Synthetic polypeptide”
YSEGTHTSDLRILLERQAIDEFIAKLLKGGGSGGGGSGGGGSGGGGSGGGGSGGGGSGGGGSGGGGSACPPC

SEQ ID NO: 429 - protein, 72 aa, Artificial Sequence
  source: /note=“Description of Artificial Sequence Synthetic polypeptide”
YSEGTHTSDLRILLERQAIDEFIAFLLKGGGSGGGGSGGGGSGGGGSGGGGSGGGGSGGGGSGGGGSACPPC

SEQ ID NO: 430 - protein, 59 aa, Artificial Sequence
  source: /note=“Description of Artificial Sequence Synthetic polypeptide”
YSEGTFISDYSIALAAAAVRDFVNWLLAGGGGGGGSGGGGSGGGGSGGGGSATHTCPPC

SEQ ID NO: 431 - protein, 59 aa, Artificial Sequence
  source: /note=“Description of Artificial Sequence Synthetic polypeptide”
YSEGTFISDYSKLLAAAAVRDFVNWLLAGGGGGGGSGGGGSGGGGSGGGGSATHTCPPC

SEQ ID NO: 432 - protein, 59 aa, Artificial Sequence
  source: /note=“Description of Artificial Sequence Synthetic polypeptide”
YSEGTFISDYSIAMDEEAVRDFVEWLKAGGGGGGGSGGGGSGGGGSGGGGSATHTCPPC

SEQ ID NO: 433 - protein, 71 aa, Artificial Sequence
  source: /note=“Description of Artificial Sequence Synthetic polypeptide”
HGEGTFTSDLSKQMEEEAVRLFIEWLKNGGPSSGAPPPSGSGGGGSGGGGSGGGGSGGGGSGGGGSACPPC

SEQ ID NO: 434 - protein, 82 aa, Artificial Sequence
  source: /note=“Description of Artificial Sequence Synthetic polypeptide”
HGEGTFTSDLSKQMEEECVRLFIEWLKNGGPSSGAPPPGCGSGGGGSGGGGSGGGGSGGGGSGGGGSGGGGSGGGGSACPPC

SEQ ID NO: 435 - protein, 72 aa, Artificial Sequence
  source: /note=“Description of Artificial Sequence Synthetic polypeptide”
YSEGTHTSDLRILLEEEAVRLFIEFLLAGGGSGGGGSGGGGSGGGGSGGGGSGGGGSGGGGSGGGGSACPPC

SEQ ID NO: 436 - protein, 59 aa, Artificial Sequence
  source: /note=“Description of Artificial Sequence Synthetic polypeptide”
YSEGTFISDYSIALAAAAQQDFVNWLLAGGGGGGGSGGGGSGGGGSGGGGSATHTCPPC

SEQ ID NO: 437 - protein, 59 aa, Artificial Sequence
  source: /note=“Description of Artificial Sequence Synthetic polypeptide”
YSEGTFISDYSIALAAAAAKDFVNWLLAGGGGGGGSGGGGSGGGGSGGGGSATHTCPPC

SEQ ID NO: 438 - protein, 59 aa, Artificial Sequence
  source: /note=“Description of Artificial Sequence Synthetic polypeptide”
YSEGTFISDYSIALAAAAIDDFVNWLLAGGGGGGGSGGGGSGGGGSGGGGSATHTCPPC

SEQ ID NO: 439 - protein, 56 aa, Artificial Sequence
  source: /note=“Description of Artificial Sequence Synthetic polypeptide”
YSEGTFTSDYSKLLEAAAAAAFAAWLAAAAGGGGGSGGGGSGGGGSGGGGSACPPC

SEQ ID NO: 440 - protein, 56 aa, Artificial Sequence
  source: /note=“Description of Artificial Sequence Synthetic polypeptide”
YSEGTFTSDYAAAAEAAAAAAFAAWLAAAAGGGGGSGGGGSGGGGSGGGGSACPPC

SEQ ID NO: 441 - protein, 62 aa, Artificial Sequence
  source: /note=“Description of Artificial Sequence Synthetic polypeptide”
YSEGTFTSDYSIALAAAAQQDFVNWLLAGGGGGGGSGGGGSGGGGSGGGGSGGGGSGGGGSA

SEQ ID NO: 442 - protein, 62 aa, Artificial Sequence
  source: /note=“Description of Artificial Sequence Synthetic polypeptide”
YSEGTFISDYSIALAAAAQQDFVNWLLKGGGGGGGSGGGGSGGGGSGGGGSGGGGSGGGGSA

SEQ ID NO: 443 - protein, 62 aa, Artificial Sequence
  source: /note=“Description of Artificial Sequence Synthetic polypeptide”
YSEGTFTSDYSIALAAAAQQDFVNWLLKGGGGGGGSGGGGSGGGGSGGGGSGGGGSGGGGSA

SEQ ID NO: 444 - protein, 56 aa, Artificial Sequence
  source: /note=“Description of Artificial Sequence Synthetic polypeptide”
YSEGTFTSDYSAAAEAAAAAAFAAWLAAAAGGGGGSGGGGSGGGGSGGGGSACPPC

SEQ ID NO: 445 - protein, 56 aa, Artificial Sequence
  source: /note=“Description of Artificial Sequence Synthetic polypeptide”
YSEGTFTSDYAAAAEAAAAAAFAAWLAKAAGGGGGSGGGGSGGGGSGGGGSACPPC

SEQ ID NO: 446 - protein, 61 aa, Artificial Sequence
  source: /note=“Description of Artificial Sequence Synthetic polypeptide”
YSEGTFISDYSIAMDKIHQQDFVNWLLAGGAAAGGSGSTASSGSGSATGGGGAATHTCPPC

SEQ ID NO: 447 - protein, 52 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSA

SEQ ID NO: 448 - protein, 62 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSGGGGSGGGGSA

SEQ ID NO: 449 - protein, 67 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSGGGGSGGGGSGGGGSA

SEQ ID NO: 450 - protein, 73 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSGGGGSGGGGSGGGGSGGGGSAN

SEQ ID NO: 451 - protein, 57 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSGGGGSA

SEQ ID NO: 452 - protein, 64 aa, Artificial Sequence
  source: /note=“Description of Artificial Sequence Synthetic polypeptide”
YSEGTFISDYSIAMDKIHQQDFVNWLLAQKPSSGAPPPSGGGGSGGGGSGGGGSGGGGSACPPC

SEQ ID NO: 453 - protein, 56 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSACPPC

SEQ ID NO: 454 - protein, 76 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSGGGGSGGGGSGGGGSGGGGSACPPC

SEQ ID NO: 455 - protein, 66 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSGGGGSGGGGSACPPC

SEQ ID NO: 456 - protein, 71 aa, Artificial Sequence
  source: /note=“Description of Artificial Sequence Synthetic polypeptide”
YSEGTFISDYSIAMDKIHQQDFVNWLLAQKGGGGGSGGGGSGGGGSGGGGSGGGGSGGGGSGGGGSACPPC

SEQ ID NO: 457 - protein, 77 aa, Artificial Sequence
  source: /note=“Description of Artificial Sequence Synthetic polypeptide”
YSEGTFISDYSIAMDKIHQQDFVNWLLAQKGAANDWKHNITQGGGGSGGGGSGGGGSGGGGSGGGGSGGGGSACPPC

SEQ ID NO: 458 - protein, 77 aa, Artificial Sequence
  source: /note=“Description of Artificial Sequence Synthetic polypeptide”
YSEGTFISDYSIAMDKIHQQDFVNWLLAQKGKKNDWKHNITQGGGGSGGGGSGGGGSGGGGSGGGGSGGGGSACPPC

SEQ ID NO: 459 - protein, 72 aa, Artificial Sequence
  source: /note=“Description of Artificial Sequence Synthetic polypeptide”
YSEGTFISDYSIAMDKIHQQDFVNWLLAQKGSGGGGSGGGGSGGGGSGGGGSGGGGSGGGGSGGGGSACPPC

SEQ ID NO: 460 - protein, 66 aa, Artificial Sequence; identical to SEQ ID NO: 455
  source: /note=“Description of Artificial Sequence Synthetic polypeptide”

SEQ ID NO: 461 - protein, 30 aa, Artificial Sequence; identical to SEQ ID NO: 8
  source: /note=“Description of Artificial Sequence Synthetic polypeptide”

SEQ ID NO: 462 - protein, 30 aa, Artificial Sequence; identical to SEQ ID NO: 8
  source: /note=“Description of Artificial Sequence Synthetic polypeptide”

SEQ ID NO: 463 - protein, 30 aa, Artificial Sequence; identical to SEQ ID NO: 190
  source: /note=“Description of Artificial Sequence Synthetic polypeptide”

(19) United States
(12) Patent Application Publication
(10) Pub. No.: US 2016/0185837 A1
(43) Pub. Date: Jun. 30, 2016

(54) GIP AND GLP-1 RECEPTOR DUAL-AGONISTS FOR THE TREATMENT OF DIABETES

(71) Applicant: Medimmune Limited, Cambridge Cambridgeshire (GB)

(72) Inventors: Maria Bednarek, Cambridge (GB); Andie Collinson, Cambridge (GB); David Hornigold, Cambridge (GB); Siobhan O'Brien, Cambridge (GB); Monika Papworth, Cambridge (GB); Peter Ravn, Cambridge (GB); Isabelle Sermadiras, Cambridge (GB)

(73) Assignee: Medimmune Limited, Cambridge Cambridgeshire (GB)

(21) Appl. No.: 14/911,910

(22) PCT Filed: Aug. 15, 2014
(86) PCT No.: PCT/EP2014/067479

§ 371 Date: Feb. 12, 2016

Related U.S. Application Data
(60) Provisional application No. 61/866,791, filed on Aug. 16, 2013.

(51) Int. Cl. C07K 14/605; C07K 16/26; A61K 45/06; A61K 38/22; A61K 39/395; A61K 9/00; C07K 14/645; A61K 38/26

(52) U.S. Cl. CPC C07K 14/605; C07K 14/645; C07K 16/26; A61K 38/26; A61K 38/2235; A61K 39/3955; A61K 9/0019; A61K 45/06; C07K 2319/30; C07K 2317/524; C07K 2317/526; C07K 2317/94; C07K 2317/71; A61K 2039/505

(57) ABSTRACT

This disclosure provides GIP/GLP-1 dual agonist polypeptides for the treatment of hypoglycemic conditions, e.g., type-2 diabetes.

### REFERENCE TO SEQUENCE LISTING SUBMITTED ELECTRONICALLY

The content of the electronically submitted sequence listing in ASCII text file (Name: 46736-134883_SL.txt; Size: 281,150 bytes; and Date of Creation: Aug. 11, 2014) filed with the application is incorporated herein by reference in its entirety.

### BACKGROUND

Diabetes mellitus type 2 (type-2 diabetes) is characterized by high blood glucose and insulin resistance. Type-2 diabetes makes up about 90% of cases of diabetes. Type-2 diabetes is frequently associated with obesity.

Incretin hormones, e.g., glucagon and glucagon-like peptide-1 are hormones that provide glycemic control during digestion. Incretin mimetics are a class of pharmacological agents currently available or in clinical trials for treatment of type-2 diabetes. Incretin mimetics have multiple antihyperglycemic actions that mimic several of the actions of incretin hormones originating in the gut, such as glucagon-like peptide (GLP)-1.

Glucagon-like peptide-1 (GLP-1) derives from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. GLP-1 is produced as a 37-amino acid peptide that corresponds to amino acids 72 through 108 of proglucagon (92 to 128 of preproglucagon). GLP-1(7-36) amide or GLP-1(7-37) acid are biologically active forms of GLP-1, that demonstrate essentially equivalent activity at the GLP-1 receptor.

GLP-1 is secreted from gut L cells and binds to the GLP-1 receptor. Its activities include stimulation of insulin synthesis and secretion, inhibition of glucagon secretion, and inhibition of food intake.

GLP-1 and GLP-1 analogs, acting as agonists at the GLP-1 receptor, have been shown to be effective hypoglycemic control, e.g., type-2 diabetes. Certain GLP-1 analogs are being sold or are in development for treatment of type-2 diabetes including, e.g., liraglutide (Victoza® from Novo Nordisk), dulaglutide (Eli Lilly), Bydureon (AZ/BMS), Aliblutide (GSK) and Exenatide (Byetta® from Eli Lilly/Amylin).

Gastric inhibitory peptide, also known as glucose-dependent insulinotropic polypeptide (GIP) is a 42-amino acid peptide hormone secreted from K cells in the intestinal epithelium. GIP secretion is regulated by food intake. GIP is also expressed in pancreatic islet α-cells and promotes insulin secretion. GIP acts at the GIP receptor, and its activities include, without limitation, stimulation of glucose-dependent insulin secretion, an increase in β-cell mass, and a decrease in gastric acid secretion.

A recent study shows that GLP-1 and GIP, alone or in combination, have shown increased expression of PDX-1, Bcl-2 and insulin in pancreatic islet cells, in both normal subjects and subjects with type-2 diabetes. Lupi et al., (2010) Regulatory peptides 165:129-132 (2010).

There remains a need for more agents for effective treatment, including improved incretin mimetics of hypoglycemic conditions such as type-2 diabetes.

### BRIEF SUMMARY

This disclosure provides an isolated polypeptide comprising or consisting of the amino acid sequence:

(SEQ ID NO: 1)

Y S X₁ G T F T S D X₂ S X₃ X₄ X₅ E X₆ X₇ X₈
X₉ X₁₀ X₁₁ X₁₂ F X₁₃ X₁₄ W L X₁₅ X₁₆
X₁₇ X₁₈;

wherein X₁ is E or Q; X₂ is Y, V, or L; X₃ is K, S, or I; X₄ is L, Y, or A; X₅ is L or M; X₆ is E or D; X₇ is E, G, or K; X₈ is E, Q, or I; X₉ is A or H; X₁₀ is V, A, or Q; X₁₁ is R, K, or Q; X₁₂ is L, E, or D; X₁₃ is I or V; X₁₄ is E, A, or N; X₁₅ is L, K, or V; X₁₆ is A, K, or N; X₁₇ is G or Q; and X₁₈ is no amino acid, G, N, P, K, or T.

In some embodiments of the polypeptide of SEQ ID NO:1, X₄ is L. In other embodiments, X₁ is E; X₂ is Y; X₃ is K; X₅ is L; X₆ is E; X₇ is E; X₈ is E; X₉ is A; X₁₀ is V; X₁₁ is R, X₁₂ is L; X₁₃ is I; X₁₄ is E; and X₁₇ is G. In other embodiments, X₁₅ is L and X₁₆ is A; X₁₅ is K and X₁₆ is N; or X₁₅ is V and X₁₆ is K.

In specific embodiments, the polypeptide comprises an amino acid sequence selected from the group consisting of SEQ ID NOs:2-32.

The invention also provides an isolated polypeptide comprising the amino acid sequence: X₁ X₂ E G T F T S D X₃ S X₄ X₅ X₆ X₇ X₈ X₉ A I D E F X₁₀ X₁₁ X₁₂ L L X₁₃ X₁₄ X₁₅ (SEQ ID NO:190); wherein X₁ is H or Y; X₂ is G or S; X₃ is V or Y; X₄ is S, K, or I; X₅ is Y, L, or A; X₆ is M or L; X₇ is E or D; X₈ is R or E; X₉ is Q or E; X₁₀ is I or V; X₁₁ is A or N; X₁₂ is W or D; X₁₃ is A, K, or G; X₁₄ is G or Q; and X₁₅ is no amino acid, G, or K.

In some embodiments, the polypeptide of SEQ ID NO:33, X₁ is Y and X₂ is S. In other embodiments, X₁₀ is I, X₁₁ is A, and X₁₂ is W. In other embodiments, X₁₄ is G and X₁₅ is no amino acid or G. In still other embodiments, X₇ is E. In other embodiments, X₈ is R and X₉ is Q. In other embodiments, X₁₃ is K. In other embodiments, X₃ is V. In still further embodiments X₄ is 5, X₅ is Y, and X₆ is M.

In specific embodiments, the polypeptide comprises an amino acid sequence selected from the group consisting of SEQ ID NOS:33-43.

The invention also provides an isolated polypeptide comprising the amino acid sequence of IP171, IP176, IP206, IP214, IP215, IP175. IP208, IP204, IP199, IP173, IP150, IP174, IP205, IP127, or IP200.

This disclosure also provides an isolated polypeptide comprising or consisting of the amino acid sequence:

(SEQ ID NO: 461)

Y S X₃ G T F T S D X₁₀ S X₁₂ X₁₃ X₁₄ X₁₅ X₁₆
X₁₇ X₁₈ X₁₉ X₂₀ X₂₁ F X₂₃ X₂₄ W L X₂₇
X₂₈ X₂₉ X₃₀;

wherein X₃ is E or Q; X₁₀ is Y, V, or L; X₁₂ is K, S, or I; X₁₃ is L, Y, or A; X₁₄ is L or M; X₁₅ is E or D; X₁₆ is E, G, or K; X₁₇ is E, Q, or I; X₁₈ is A or H; X₁₉ is V, A, or Q; X₂₀ is R, K, or Q; X₂₁ is L, E, or D; X₂₃ is I or V; X₂₄ is E, A, or N; X₂₇ is L, K, or V; X₂₈ is A, K, or N; X₂₉ is G or Q; and X₃₀ is no amino acid, G, N, P, K, or T.

In some embodiments of the polypeptide of SEQ ID NO:461, X₁₃ is L. In other embodiments, X₃ is E; X₁₀ is Y; X₁₂ is K; X₁₄ is L; X₁₅ is E; X₁₆ is E; X₁₇ is E; X₁₈ is A; X₁₉ is V; X₂₀ is R, X₂₁ is L; X₂₃ is I; X₂₄ is E; and X₂₉ is G. In other embodiments, X₂₇ is L and X₂₈ is A; X₂₇ is K and X₂₈ is N; or X₂₇ is V and X₂₈ is K. In specific embodiments, the polypeptide comprises an amino acid sequence selected from the group consisting of SEQ ID NOs:2-32.

This disclosure also provides an isolated polypeptide comprising or consisting of the amino acid sequence:

(SEQ ID NO: 462)

X₁ X₂ X₃ G X₅ X₆ X₇ S X₉ X₁₀ X₁₁
X₁₂ X₁₃ X₁₄ X₁₅ X₁₆ X₁₇ X₁₈ X₁₉ X₂₀
X₂₁ F X₂₃ X₂₄ X₂₅ L X₂₇ X₂₈ X₂₉ X₃₀;

wherein X₁ is Y or H; X₂ is S or G; X₃ is E or Q; X₅ is T or M; X₆ if F or H; X₇ is T or I; X₉ is D or L; X₁₀ is Y, V, F, or L; X₁₁ is S, R, or M; X₁₂ is K, S, M, or I; X₁₃ is L, Y, Q, H, I or A; X₁₄ is L, K, or M; X₁₅ is E or D; X₁₆ is E, G, or K; X₁₇ is E, Q, or I; X₁₈ is A, C, or H; X₁₉ is V, A, E, I, N, S, T, or Q; X₂₀ is R, K, or Q; X₂₁ is L, E, C, or D; X₂₃ is I or V; X₂₄ is E, C, A, or N; X₂₅ is W, C, F, H, Y, or K; X₂₇ is L, K, or V; X₂₈ is A, K, C, or N; X₂₉ is G or Q; and X₃₀ is no amino acid, G, N, P, K, or T. In specific embodiments, the polypeptide comprises an amino acid sequence selected from the group consisting of SEQ ID NOs:2-32 and 191-256.

The disclosure also provides an isolated polypeptide comprising the amino acid sequence:

(SEQ ID NO: 463)

X₁ X₂ E G X₅ X₆ X₇ S X₉ X₁₀ X₁₁ X₁₂ X₁₃ X₁₄
X₁₅ X₁₆ X₁₇ A I X₂₀ X₂₁ F X₂₃ X₂₄ X₂₅ L X₂₇
X₂₈ X₂₉ X₃₀;

wherein X₁ is H or Y; X₂ is G or S; X₅ is T or M; X₆ is F or H; X₇ is T or I; X₉ is D or L; X₁₀ is V, F, L, or Y; X₁₁ is S, A, or R; X₁₂ is S, K, A, M, Q or I; X₁₃ is Y, L, H, or A; X₁₄ is M, A, K, I, or L; X₁₅ is E or D; X₁₆ is R, K, or E; X₁₇ is Q or E; X₂₀ is D or E; X₂₁ is E or A; X₂₃ is I, A, or V; X₂₄ is A or N; X₂₅ is W, E, L, F, I, S, N, G, M, H, K or D; X₂₇ is L or A; X₂₈ is A, K, or G; X₂₉ is G, A, or Q; and X₃₀ is no amino acid, G, A, E, or K. In specific embodiments, the polypeptide comprises an amino acid sequence selected from the group consisting of SEQ ID NOS:33-43 and 257-294.

The polypeptides of the invention may further a heterologous polypeptide fused thereto. In some embodiments, the heterologous polypeptide comprises a linker, a hinge, an Fc domain, or a combination thereof. In some embodiments, the linker comprises (GGGGS)n, wherein n 1, 2, 3, or 4. In certain specific embodiments, the linker comprises the amino acid sequence: G GGGGS GGGGS GGGGS GGGGS A (SEQ ID NO:66), A PPGGS GGGGS GGGGS GGGGS A (SEQ ID NO:67), GT GGGGS GGGGS GGGGS GGGGS A (SEQ ID NO:68), G GGGGS GGGGS GGGGS GGGGS A (SEQ ID NO:69), G GGGGS A (SEQ ID NO:70), G GGGGS GGGGS A (SEQ ID NO:71), G GGGGS GGGGS GGGGS A (SEQ ID NO:72), G KGGGS GGGGS GGGGS GGGGS A (SEQ ID NO:73), G GGGGS GGGGS GGGGS GGGGSA (SEQ ID NO:179), G GGGG GGGG GGGG GGGG A (SEQ ID NO:180) or any combination, fragment or variant thereof.

In some embodiments, the linker comprises GG SGSTA SSGSG SATGG GGAA (SEQ ID NO:74), any variant thereof, or any fragment thereof. In other embodiments, the linker comprises AAAGG SGSTA SSGSG SATGG GGAA (SEQ ID NO:75), APPGG SGSTA SSGSG SATGG GGAA (SEQ ID NO:76), or any combination, fragment or variant thereof.

In some embodiments, the hinge comprises an amino acid sequence of an IgG1 hinge, an IgG4 hinge, a fragment thereof, a variant thereof, or any combination thereof. In certain specific embodiments, the hinge comprises ESKYGPPCPPCPAPEAA (SEQ ID NO:77), THTCPPCPAPEF (SEQ ID NO:78), THTCPPC (SEQ ID NO:79), CPPCPAPEF (SEQ ID NO:80), TYTCPPCPAPEF (SEQ ID NO:81), TSTCPPCPAPEF (SEQ ID NO:82), PPCPPCPAPEF (SEQ ID NO:83), ESKYGPPCPPCPAPEF (SEQ ID NO:84), APEF (SEQ ID NO:85), ESKYGPPCPPC (SEQ ID NO:86), THTCPPCPAPELL (SEQ ID NO:87), any variant thereof, any fragment thereof, or any combination thereof.

In some embodiments, the Fc region comprises an IgG1 Fc region, an IgG1-TM Fc region, an IgG1-FQQ Fc region, an IgG4 Fc region, an IgG1-YTE Fc region, any fragment thereof, any variant thereof, or any combination thereof.

In certain specific embodiments, the polypeptide and heterologous polypeptide comprises the amino acid sequence selected from the group consisting of: SEQ ID NOS:88-175. In a specific embodiment, the polypeptide and heterologous polypeptide comprises IP088 (SEQ ID NO:105).

The polypeptides of the invention (with or without the heterologous polypeptide) may further comprise a heterologous moiety attached thereto.

In some embodiments, the heterologous moiety is a polypeptide, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, or a combination of two or more of the recited moieties.

In some embodiments of the invention, the polypeptide binds to a GIP receptor, a GLP-1 receptor, or to both a GIP and a GLP-1 receptor. In certain embodiments, the polypeptide binds to a GIP receptor. For example, but not by way of limitation, the GIP receptor may be a mouse GIP receptor, a rat GIP receptor, or a human GIP receptor.

In some embodiments, the polypeptide binds to a human GIP receptor with an EC50 in the cAMP assay 1 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In some embodiments, the polypeptide binds to a human glucagon receptor with at least 1000-fold lower affinity than its binding affinity for a human GIP receptor, as measured in the cAMP assay.

In some embodiments, the polypeptide of the invention binds to a GLP-1 receptor. For example, but not by way of limitation, the GLP-1 receptor is a mouse GLP-1 receptor, a rat GLP-1 receptor, or a human GLP-1 receptor.

In some embodiments, the polypeptide binds to a human GLP-1 receptor with an EC50 in the cAMP assay 1 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In some embodiments, the polypeptide binds to a human glucagon receptor with at least 1000-fold lower affinity than its binding affinity for a human GLP-1 receptor, as measured in the cAMP assay.

In some embodiments, the polypeptide of the invention is an agonist of GLP-1 activity, an agonist of GIP activity, or an agonist of both GLP-1 and GIP activity.

The invention also provides an isolated polynucleotide encoding the polypeptide of any one of the polypeptides of the invention. The invention also provides a vector comprising the polynucleotides of the invention and host cells comprising the polynucleotides of the invention, optionally operably connected to a vector.

The invention also provides a method of making a polypeptide of the invention comprising culturing a host cell containing a polynucleotide of the invention under conditions allowing expression of the polypeptide, and recovering the polypeptide. In some embodiments, the polynucleotide is operably linked to a vector.

The invention further provides a pharmaceutical composition comprising a polypeptide of the invention and a carrier and kits comprising such a pharmaceutical composition.

The invention further provides a method of treating or preventing a disease or condition caused or characterized by hypoglycemia or impaired insulin release, comprising administering to a subject in need of treatment an effective amount of at least one polypeptide of the invention or a composition of the invention.

In some embodiments, the disease or condition is diabetes, such as, for example, Type-2 diabetes.

In some embodiments of the method of treating or preventing a disease or condition caused or characterized by hypoglycemia or impaired insulin release, the administration further improves glycemic control, provides body weight control, improves β-cell function and mass, reduces the rate of gastric acid secretion and gastric emptying, or any combination thereof.

In some embodiments of the method of treating or preventing a disease or condition caused or characterized by hypoglycemia or impaired insulin release, the peptide is administered orally or by injection (e.g., subcutaneously or intravenously). In some embodiments, the polypeptide is administered once per day. In some embodiments, the method further comprises administering one or more additional therapies. For example, but not by way of limitation, the additional therapy comprises blood sugar monitoring, diet modifications, exercise, insulin, a thiazolidinedione, a sulfonylurea, an incretin, metformin, a glyburide, a dipeptidyl peptidase 4 inhibitor, a bile acid sequestrant, or any combination thereof. In some embodiments, the subject is human.

### BRIEF DESCRIPTION OF THE DRAWINGS/FIGURES

FIG. 1 shows the structure of an exemplary GIP/GLP-1 agonist polypeptide as provided herein.

FIG. 2A shows an alignment of exendin-4 (1-30) (SEQ ID NO:187), human GLP-1 (SEQ ID NO:176), human GIP (1-30) (SEQ ID NO:188), GIP/GLP-1 agonist polypeptide portion of IP088 (SEQ ID NO:15), and the GLP-1 analog Fc control (GLP1aFc) (SEQ ID NO:189).

FIGS. 2B and 2C show potency curves for IP088 on hGLP-1r- and GIPr-expressing cells. The curve is compared with hGLP-1 in FIG. 2B and with hGIP in FIG. 2C. Both panels show a comparison to a GLP-1 analogue-Fc fusion control (GLP-1aFc).

FIG. 2D is a curve showing pharmacokinetics of IP088 (circles) in mouse out to 48 hours, compared to GLP-1aFc (inverted triangles).

FIG. 3A-C shows the pharmacokinetic profile of IP088 (panel A) and an GLP-1 analogue-Fc (gamma4) fusion control (GLP-1aFc) (panel B) in mouse over 168 hours as measured by the presence of human IgG1 Fc by ELISA. Circles: IV injection of 1 mg/kg, squares: subcutaneous injection of 1 mg/kg. Pharmacokinetic parameters Cₘₐₓ, bioavailability (F %) and half-life T_{1/2} are compared in panel C.

FIG. 3D-F shows the pharmacokinetic activity of IP088 (panel D) and GLP-1aFc (panel E) in mouse over 168 hours, as measured by potency in a cAMP reporter activity assay on cells expressing human GLP-1r. Circles: IV injection of 1 mg/kg, squares: subcutaneous injection of 1 mg/kg. Pharmacokinetic parameters Cₘₐₓ, bioavailability (F %) and half life T₁₁₂ are compared in panel F.

FIG. 4A shows a schematic of the oral glucose tolerance tests carried out in C57 mice.

FIG. 4B shows the results of the OGTT on day 0. □=IP040 (vehicle only), =liraglutide, *=GLP-1aFc (Peptide SEQ ID NO:62-Fc), and ⋄=IP088.

FIG. 4C shows the results of the OGTT on day 2. □=IP040 (vehicle only), =liraglutide, *=GLP-1aFc (Peptide SEQ ID NO:62-Fc), and ⋄=IP088.

### DETAILED DESCRIPTION

### Definitions

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to whom this disclosure is directed. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides that have undergone post-translational or post-synthesis modifications, for example, glycosylation, acetylation, phosphorylation, amidation, derivatization by known protecting/blocking groups, proteolytic cleavage, or modification by non-naturally occurring amino acids.

More specifically, the term “polypeptide” as used herein encompasses full length peptides and fragments, variants or derivatives thereof, e.g., a GIP/GLP-1 agonist polypeptide (e.g., 29, 30, or 31 amino acids in length). A “polypeptide” as disclosed herein, e.g., a GIP/GLP-1 agonist polypeptide, can comprise a fusion polypeptide comprising one or more additional components such as, e.g., a linker, a hinge, an Fc domain or an albumin domain, to increase half-life, impart flexibility, allow for dimerization or other desired properties. A polypeptide as described herein can also be derivatized in a number of different ways.

A polypeptide as provided herein can be multimeric. As used herein, the terms “multimer,” “multimeric” and “multivalent” refer to a molecule, e.g., a GIP/GLP-1 agonist polypeptide, that comprises at least GIP/GLP-1 agonist polypeptides in association. The multimer, e.g., a dimer, trimer, tetramer, or larger polypeptide, can be linked through a disulfide bonds, hydrogen bonds, or other covalent or non-covalent linkages.

The terms “fragment,” “analog,” “derivative,” or “variant” when referring to a GIP/GLP-1 agonist polypeptide includes any polypeptide that retains at least some desirable activity, e.g., binding to GIP and/or GLP-1 receptors. Fragments of GIP/GLP-1 agonist polypeptides provided herein include proteolytic fragments, deletion fragments that exhibit desirable properties during expression, purification, and or administration to a subject.

The term “variant,” as used herein, refers to a polypeptide that differs from the recited polypeptide due to amino acid substitutions, deletions, insertions, and/or modifications. Variants can be produced using art-known mutagenesis techniques. Variants can also, or alternatively, contain other modifications—for example a polypeptide can be conjugated or coupled, e.g., fused to a heterologous amino acid sequence or other moiety, e.g., for increasing half-life, solubility, or stability. Examples of moieties to be conjugated or coupled to a polypeptide provided herein include, but are not limited to a linker, a hinge, albumin, an immunoglobulin Fc region, polyethylene glycol (PEG), and the like. The polypeptide can also be conjugated or produced coupled to an element for ease of synthesis, purification or identification of the polypeptide (e.g., 6-His), or to enhance binding of the polypeptide to a solid support.

The term “sequence identity” as used herein refers to a relationship between two or more polynucleotide sequences or between two or more polypeptide sequences. When a position in one sequence is occupied by the same nucleic acid base or amino acid in the corresponding position of the comparator sequence, the sequences are said to be “identical” at that position. The percentage “sequence identity” is calculated by determining the number of positions at which the identical nucleic acid base or amino acid occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a polynucleotide or polypeptide sequence in the comparison window can comprise additions or deletions termed gaps while the reference sequence is kept constant. An optimal alignment is that alignment that, even with gaps, produces the greatest possible number of “identical” positions between the reference and comparator sequences. Percentage “sequence identity” between two sequences can be determined using the version of the program “BLAST 2 Sequences” that was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap drop-off (50), expect value (10), and any other required parameter including but not limited to matrix option.

The terms “polynucleotide” or “nucleotide” as used herein are intended to encompass a singular nucleic acid as well as plural nucleic acids, and refers to an isolated nucleic acid molecule or construct, e.g., messenger RNA (mRNA) or plasmid DNA (pDNA). In certain aspects, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA or RNA fragments, present in a polynucleotide. When applied to a nucleic acid or polynucleotide, the term “isolated” refers to a nucleic acid molecule, DNA or RNA that has been removed from its native environment, for example, a recombinant polynucleotide encoding an polypeptide comprising a variant Fc domain contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

The term “vector” means a construct that is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, the term “host cell” refers to a cell or a population of cells harboring or capable of harboring a recombinant nucleic acid. Host cells can be a prokaryotic cells (e.g., E. coli), or alternatively, the host cells can be eukaryotic, for example, fungal cells (e.g., yeast cells such as Saccharomyces cerivisiae, Pichia pastoris, or Schizosaccharomyces pombe), and various animal cells, such as insect cells (e.g., Sf-9) or mammalian cells (e.g., HEK293F, CHO, COS-7, NIH-3T3).

The terms “composition” or “pharmaceutical composition” refer to compositions containing a polypeptide comprising a GIP/GLP-1 agonist polypeptide provided herein, along with e.g., pharmaceutically acceptable carriers, excipients, or diluents for administration to a subject in need of treatment, e.g., a human subject being treated for a hypoglycemic condition, e.g., type-2 diabetes.

The term “pharmaceutically acceptable” refers to compositions that are, within the scope of sound medical judgment, suitable for contact with the tissues of human beings and animals without excessive toxicity or other complications commensurate with a reasonable benefit/risk ratio.

An “effective amount” is that amount of a polypeptide comprising a GIP/GLP-1 agonist polypeptide provided herein, the administration of which to a subject, either in a single dose or as part of a series, is effective for treatment, e.g., treatment of type-2 diabetes. An amount is effective, for example, when its administration results in one or more of prevention or modulation of hyperglycemia, promotion of insulin synthesis, an increase in β-cell mass, weight loss or weight maintenance (e.g., prevention of weight gain), reduction in food intake, modulation of gastric acid secretion, or modulation of gastric emptying. This amount can be a fixed dose for all subjects being treated, or can vary depending upon the weight, health, and physical condition of the subject to be treated, the extent of glycemic control desired, the formulation of polypeptide, a professional assessment of the medical situation, and other relevant factors.

The term “subject” is meant any subject, particularly a mammalian subject, in need of treatment with a GIP/GLP-1 agonist polypeptide provided herein. Mammalian subjects include, but are not limited to, humans, dogs, cats, guinea pigs, rabbits, rats, mice, horses, cattle, bears, cows, apes, monkeys, orangutans, and chimpanzees, and so on. In one aspect, the subject is a human subject.

As used herein, an “subject in need thereof” refers to an individual for whom it is desirable to treat, e.g., a subject diagnosed with a hypoglycemic condition, e.g., type-2 diabetes, or a subject prone to contract a hypoglycemic condition, e.g., type-2 diabetes.

As used herein a “GIP/GLP-1 agonist polypeptide” is a chimeric polypeptide that exhibits activity at the GIP receptor of at least about 1%, 5%, 10%, 20%, 30%, 40%, 50%, 60%, 70%, 80%, 90%, 95%, or more (up to 100%) relative to native GIP and also exhibits activity at the GLP-1 receptor of about at least about 1%, 5%, 10%, 20%, 30%, 40%, 50%, 60%, 70%, 80%, 90%, 95%, or more (up to 100%) relative to native GLP-1, under conditions provided elsewhere herein, e.g., the cAMP assay performed in Hanks Balanced Salt Solution supplemented with bovine serum albumin (“BSA-cAMP assay”), described in Example 2.

As used herein the term “native GIP” refers to naturally-occurring GIP, e.g., human GIP (i.e., GIP₁₋₄₂), comprising the sequence of SEQ ID NO:178, or an active fragment thereof. The term “native GLP-1” refers to naturally-occurring GLP-1, e.g., human GLP-1, and is a generic term that encompasses, e.g., GLP-1(7-36) amide (SEQ ID NO:176), GLP-1(7-37) acid (SEQ ID NO:177) or active fragments thereof, or a mixture of those two compounds. As used herein, a general reference to “GIP” or “GLP-1” in the absence of any further designation is intended to mean native human GIP or native human GLP-1, respectively. Unless otherwise indicated, “GIP” refers to human GIP, and “GLP-1” refers to human GLP-1.

### GIP/GLP-1 Agonist Polypeptides

Provided herein are polypeptides that bind both to a GIP receptor and to a GLP-1 receptor. In certain aspects, the peptides provided herein are dual-agonists of GIP and GLP-1 activity. Such peptides are referred to herein as GIP/GLP-1 agonist polypeptides. GIP/GLP-1 agonist polypeptides as provided herein possess GLP-1 and GIP activities with favorable ratios to promote enhanced control of a hypoglycemic condition, e.g., type-2 diabetes. Polypeptides as provide herein can promote one or more of glycemic control, increased insulin production, decreased glucagon production, increased β-cell mass, or decreased body fat. Polypeptides provided herein can further possess optimized solubility, formulatability, and stability. In certain aspects, GIP/GLP-1 agonist polypeptides as provided herein are active at the human GLP-1 and human GIP receptors. In certain aspects, GIP/GLP-1 agonist polypeptides as provided herein are also active at rodent GLP-1 and rodent GIP receptors, e.g., rat or mouse GLP-1 receptor or rat or mouse GIP receptor. In certain aspects, GIP/GLP-1 agonist polypeptides as provided herein are also active at non-human primate GLP-1 and GIP receptors, e.g., cynomolgus monkey GLP-1 and GIP receptors.

Certain GIP/GLP-1 agonist peptides provided herein are fusion proteins comprising a GIP/GLP-1-like peptide domain, and one or more additional domains including, but not limited to one or more of a linker, a hinge, or an Fc domain. Suitable linkers, hinges, and Fc domains are described elsewhere in this disclosure. Additional linkers, hinges, and Fc domains are well-known to those of ordinary skill in the art and can be incorporated into GIP-GLP-1 agonist polypeptides as described herein, and tested for potency, activity, and efficacy in treating hypoglycemic conditions, e.g., type-2 diabetes without undue experimentation according to the methods provided herein.

In one aspect, this disclosure provides a GIP/GLP-1 agonist polypeptide comprising a peptide sequence derived from G89. The G89 peptide (SEQ ID NO:2) was derived by introducing amino acid substitutions into a truncated version of exendin-4 (SEQ ID NO:187), a GLP-1-like peptide derived from Heloderma suspectum venom (commercially available under the generic name exenatide, see U.S. Pat. No. 5,424,286, incorporated herein by reference in its entirety). G89 was identified by cAMP potency assays on cells expressing GIPr and cells expressing GLP-1r and has 7 amino acid substitutions relative to amino acids 1 to 29 of exendin-4.

According to this aspect, this disclosure provides an isolated polypeptide comprising the amino acid sequence:

(SEQ ID NO: 1)

Y S X₁ G T F T S D X₂ S X₃ X₄ X₅ E X₆ X₇ X₈
X₉ X₁₀ X₁₁ X₁₂ F X₁₃ X₁₄ W L X₁₅ X₁₆ X₁₇ X₁₈;

where X₁ is E or Q; X₂ is Y, V, or L; X₃ is K, S, or I; X₄ is L, Y, or A; X₅ is L or M; X₆ is E or D; X₇ is E, G, or K; X₈ is E, Q, or I; X₉ is A or H; X₁₀ is V, A, or Q; X₁₁ is R, K, or Q; X₁₂ is L, E, or D; X₁₃ is I or V; X₁₄ is E, A, or N; X₁₅ is L, K, or V; X₁₆ is A, K, or N; X₁₇ is G or Q; and X₁₈ is no amino acid, G, N, P, K, or T. In certain aspects, X₄ is L. In certain aspects, X₁ is E; X₂ is Y; X₃ is K; X₄ is L; X₅ is L; X₆ is E; X₇ is E; X₈ is E; X₉ is A; X₁₀ is V; X₁₁ is R, X₁₂ is L; X₁₃ is I; X₁₄ is E; and X₁₇ is G. In further aspects, related to any of the aspects noted above, X₁₅ is L and X₁₆ is A; X₁₅ is K and X₁₆ is N; or X₁₅ is V and X₁₆ is K.

This aspect of the disclosure also provides an isolated polypeptide comprising or consisting of the amino acid sequence:

(SEQ ID NO: 461)

Y S X₃ G T F T S D X₁₀ S X₁₂ X₁₃ X₁₄ X₁₅ X₁₆
X₁₇ X₁₈ X₁₉ X₂₀ X₂₁ F X₂₃ X₂₄
W L X₂₇ X₂₈ X₂₉ X₃₀;

wherein X₃ is E or Q; X₁₀ is Y, V, or L; X₁₂ is K, S, or I; X₁₃ is L, Y, or A; X₁₄ is L or M; X₁₅ is E or D; X₁₆ is E, G, or K; X₁₇ is E, Q, or I; X₁₈ is A or H; X₁₉ is V, A, or Q; X₂₀ is R, K, or Q; X₂₁ is L, E, or D; X₂₃ is I or V; X₂₄ is E, A, or N; X₂₇ is L, K, or V; X₂₈ is A, K, or N; X₂₉ is G or Q; and X₃₀ is no amino acid, G, N, P, K, or T. In some embodiments of the polypeptide of SEQ ID NO:461, X₁₃ is L. In other embodiments, X₃ is E; X₁₀ is Y; X₁₂ is K; X₁₄ is L; X₁₅ is E; X₁₆ is E; X₁₇ is E; X₁₈ is A; X₁₉ is V; X₂₀ is R, X₂₁ is L; X₂₃ is I; X₂₄ is E; and X₂₉ is G. In other embodiments, X₂₇ is L and X₂₈ is A; X₂₇ is K and X₂₈ is N; or X₂₇ is V and X₂₈ is K.

This aspect of the disclosure also provides an isolated polypeptide comprising or consisting of the amino acid sequence:

(SEQ ID NO: 462)

X₁ X₂ X₃ G X₅ X₆ X₇ S X₉ X₁₀ X₁₁ X₁₂ X₁₃
X₁₄ X₁₅ X₁₆ X₁₇ X₁₈ X₁₉ X₂₀ X₂₁
F X₂₃ X₂₄ X₂₅ L X₂₇ X₂₈ X₂₉ X₃₀ ;

wherein X₁ is Y or H; X₂ is S or G; X₃ is E or Q; X₅ is T or M; X₆ if F or H; X₇ is T or I; X₉ is D or L; X₁₀ is Y, V, F, or L; X₁₁ is S, R, or M; X₁₂ is K, S, M, or I; X₁₃ is L, Y, Q, H, I or A; X₁₄ is L, K, or M; X₁₅ is E or D; X₁₆ is E, G, or K; X₁₇ is E, Q, or I; X₁₈ is A, C, or H; X₁₉ is V, A, E, I, N, S, T, or Q; X₂₀ is R, K, or Q; X₂₁ is L, E, C, or D; X₂₃ is I or V; X₂₄ is E, C, A, or N; X₂₅ is W, C, F, H, Y, or K; X₂₇ is L, K, or V; X₂₈ is A, K, C, or N; X₂₉ is G or Q; and X₃₀ is no amino acid, G, N, P, K, or T.

In certain specific aspects, this disclosure provides a G89-related GIP/GLP-1 agonist polypeptide comprising any one or more of the amino acid sequences listed in Table 1.

TABLE 1
GIP/GLP-1 Agonist Peptides based on G89

| | SEQ |
|---|---|
| | ID |
| Peptide | NO |
| YSEGT FTSDY SKLLE EEAVR LFIEW LLAG (G89) | 2 |
| YSEGT FTSDV SKLLE EEAVR LFIEW LLAG | 3 |
| YSEGT FTSDY SKLLD EEAVR LFIEW LLAG | 4 |
| YSEGT FTSDY SKLLE EEAVR LFIEW LVKG | 5 |
| YSEGT FTSDY SKLLE EEAVR LFIEW LLAGN | 6 |
| YSEGT FTSDY SKLLE EEAVR LFIEW LLAGG HNITQ | 7 |
| YSEGT FTSDY SKLLE EEAVR LFIEW LLAGG | 8 |
| YSEGT FTSDY SKLLE EEAVR LFIEW LLAGP | 9 |
| YSEGT FTSDY SKLLE EEAVR LFIEW LLAGP SSGAP PPS | 10 |
| YSEGT FTSDY SKLLE EEAVR LFIEW LLAQK | 11 |
| YSEGT FTSDY SKLLE EEAVR LFIEW LKNGG | 12 |
| YSEGT FTSDY SKLLE EEAVR LFIEW LKNGG PSSGA PPPS | 13 |
| YSEGT FTSDY SKLLE EEAVR LFIEW LKNGT | 14 |
| YSEGT FTSDY SKLLE EEAVR LFIEW LVKGP | 15 |
| YSQGT FTSDY SKLLE EEAVR LFIEW LVKGP | 16 |
| YSEGT FTSDL SKLLE EEAVR LFIEW LVKGP | 17 |
| YSEGT FTSDY SSYME EEAVR LFIEW LVKGP | 18 |
| YSEGT FTSDY SIAMD EEAVR LFIEW LVKGP | 19 |
| YSEGT FTSDY SKLLE GQAAK LFIEW LVKGP | 20 |
| YSEGT FTSDY SKLLE KIHQQ LFIEW LVKGP | 21 |
| YSEGT FTSDY SKLLE EEAQR LFIEW LVKGP | 22 |
| YSEGT FTSDY SKLLE EEAVR EFIAW LVKGP | 23 |
| YSEGT FTSDY SKLLE EEAVR DFVNW LVKGP | 24 |
| _SEGT FTSDY SKLLE EEAVR LFIEW LVKGP | 25 |
| YSEGT FTSDY SKLLD EEAVR LFIEW LVKGP | 26 |
| YSEGT FTSDY SKLLE KEAVR LFIEW LVKGP | 27 |
| YSEGT FTSDY SKLLE EEAVQ LFIEW LVKGP | 28 |
| YSEGT FTSDY SKLLE EEAVR LFVEW LVKGP | 29 |
| YSEGT FTSDY SKLLE EEAVR LFIEW LLKGP | 30 |
| YSEGT FTSDY SKLLE EEAVR LFIEW LVKQP | 31 |
| YSEGT FTSDY SKLLE EEAVR LFIEW LVKGK | 32 |
| YSEGT FTSDY SKLLE EEAVR LFIEW LLAG | 191 |
| YSEGT FTSDY SKLLE EEHVR LFIEW LVKGP | 192 |
| YSEGT FTSDY SKLLE EEAVR LFIEW LVKGP | 193 |
| YSEGT FTSDY SKLLE EEAVR LFIEW LLAQK | 194 |
| YSEGT FTSDY SKLLE EEAVR LFIEW LVKGP | 195 |
| YSEGT FTSDY SKLLE KEAVR LFIEW LLKGP | 196 |
| YSEGT FTSDY SKLLE KEAQR LFIEW LLKGP | 197 |
| YSEGT FTSDY SKLLE EEAVR LFIEW LVKGP | 198 |
| YSEGT FTSDY SKLLE EEAVR LFIEW LVKGP PSSGA PPPS | 199 |
| YSEGT FTSDY SKLLE EEAVR LFIEW LVKGP SSGAP PPS | 200 |
| YSEGT FTSDY SKLLE EEAVR LFIEW LVKGG PSSGA PPPS | 201 |
| YSEGT FTSDY SKLLE EEAVR LFIEW LVKGP | 202 |
| YSEGT FTSDY SKLLE EEAVR LFIEW LVKGP | 203 |
| YSEGT FTSDY SKLLE EEAVR LFIEW LVKGP | 204 |
| YSEGT FTSDY SKLLE EEAVR LFIEW LVKGP | 205 |
| YSEGT FTSDY SKLLE EEAVR LFIEW LVKGP | 206 |
| YSEGT FTSDY SKLLE KEAER LFIEW LLKGP | 207 |
| YSEGT FTSDY SKLLE EEAVR LFIEW LVKGP | 208 |
| YSEGT FTSDY SKLLE EECVR LFIEW LVKGG PGGGA PPPGC | 209 |
| YSEGT FTSDY SKLLE EEAVR CFIEW LVKGG PGGGA PPCGG | 210 |
| YSEGT FTSDY SKLLE EEAVR CFIEC LVKGP | 211 |
| YSEGT FTSDY SKLLE EEAVR LFICW LVCGP | 212 |
| YSEGT FTSDY SKLLE EEAVR LFIEW LLAGG PSSGA PPPS | 213 |
| YSEGT FTSDY SKLLE EEAVR CFIEW LVKGG PSSGA PPCS | 214 |
| YSEGT FTSDY SKLLE EEAVR LFIEW LLAG | 215 |
| YSEGT FISDY SKLLE EEAVR DFVEW LKAGG | 216 |
| YSEGT FTSDY SKLLE EEAVR LFIEW LLAG | 217 |
| YSEGT FTSDY SKLLE EEAVR LFIEW LVKGP | 218 |
| YSEGT FTSDY SKLLE EEAVR LFIEW LVKGP | 219 |
| YSEGT FTSDY SKLLE EEAVR LFIEW LVKGP | 220 |
| YSEGT FTSDY SKLLE EEAVR LFIEW LVKGP | 221 |
| YSEGT FTSDY SKLLE EEAVR LFIEW LLAGG | 222 |
| YSEGT FTSDY SKLLE EEAVR LFIEW LLAGG | 223 |
| YSEGT FTSDY SKLLE EEAVR LFIEW LLAGG | 224 |
| YSEGT FTSDY SKLLE KEAQR LFIEW LLAGG | 225 |
| HGEGT FTSDL SKQME EEAVR LFIEW LKNGG PSSGA PPPS | 226 |
| HGEGT FTSDL SKQME EEAVR LFIEW LKNGG PSSGA PPPS | 298 |
| HGEGT FTSDL SKQME EECVR LFIEW LKNGG PSSGA PPPGC | 299 |
| YSEGM HTSDF SKLLE EEAVR LFIEW LLAGG | 227 |
| YSEGT HTSLF SKLLE EEAVR LFIEW LLAGG | 228 |
| YSEGT FTSDF SKLLE EEAVR LFIEF LLAGG | 229 |
| YSEGT FTSDF SKLLE EEAVR LFIEW LLAGG | 230 |
| YSEGT FTSDY SKLLE EEAVR LFIEH LLAGG | 231 |
| YSEGT FTSDY RILLE EEAVR LFIEW LLAGG | 232 |
| YSEGT FTSDY RILKE EEAVR LFIEW LLAGG | 233 |
| YSEGT FTSDY MMLKE EEAVR LFIEW LLAGG | 234 |
| YSEGT FTSDY RMLKE EEAVR LFIEW LLAGG | 235 |
| YSEGT FTSLY SKLKE EEAVR LFIEW LLAGG | 236 |
| YSEGM HTSDL SKLLE EEAVR LFIEW LLAGG | 237 |
| YSEGT FTSDY SIYLD EEAVR LFIEW LLAGG | 238 |
| YSEGT FTSLY SKLLE EEAVR LFIEW LLAGG | 239 |
| YSEGT HTSDF SKLLE EEAVR LFIEW LLAGG | 240 |
| YSEGT FTSDY SKLLE EEAVR LFIEF LLAGG | 241 |
| YSEGT FTSDY SKLLE EEAVR LFIEY LLAGG | 242 |
| YSEGT FTSLY SKLLE EEAVR LFIEY LLAGG | 243 |
| YSEGT FTSLY RILLE EEAVR LFIEW LLAGG | 244 |
| YSEGT FTSDY SKLLE EEAVR LFIEW LLKGG | 245 |
| YSEGT FTSDY SKLLE EEAVR LFIEW LVKGP | 246 |
| YSEGT HTSDL SKLLE EEAVR LFIEW LLAGG | 247 |
| YSEGT HTSDL RILLE EEAVR LFIEK LLAGG | 248 |
| YSEGT FTSDY SKLLE EEAIR LFIEW LLAGG | 249 |
| YSEGT FTSLY RIHKE EEAVR LFIEK LLAGG | 250 |
| YGEGT FTSDY SKLLE EEAVR LFIEW LVKGP | 251 |
| YSEGT FTSDY SKLLE EEANR LFIEW LLAGG | 252 |
| YSEGT FTSDY SKLLE EEASR LFIEW LLAGG | 253 |
| HSQGT FTSDY SKILE EEAVR LFIEW LLAGG | 254 |
| YSEGT FTSDY SKLLE EEAVR LFIEW LLAGG | 255 |
| YSEGT FTSDY SKLLE EEATR LFIEW LLAGG | 256 |

In further aspects, this disclosure provides a GIP/GLP-1 agonist polypeptide comprising a peptide sequence derived from the “AID” peptide (SEQ ID NO:33). The AID peptide was selected from a phage display library of peptides based on GLP-1, by binding to GLP-1r and GIPr and confirmed in a cAMP selectivity assay. AID has 7 amino acid substitutions relative to human GLP-1 (1-29).

According to this aspect, this disclosure provides an isolated polypeptide comprising the amino acid sequence:

(SEQ ID NO: 190)

X₁ X₂ E G T F T S D X₃ S X₄ X₅ X₆ X₇
X₈ X₉ A I D E F X₁₀ X₁₁ X₁₂ L L X₁₃ X₁₄ X₁₅;

where X₁ is H or Y; X₂ is G or S; X₃ is V or Y; X₄ is S, K, or I; X₅ is Y, L, or A; X₆ is M or L; X₇ is E or D; X₈ is R or E; X₉ is Q or E; X₁₀ is I or V; X₁₁ is A or N; X₁₂ is W or D; X₁₃ is A, K, or G; X₁₄ is G or Q; and X₁₅ is no amino acid, G, or K. In certain aspects, X₁ is Y and X₂ is S. In certain aspects, X₁₀ is I, X₁₁ is A, and X₁₂ is W; or X₁ is Y, X₂ is S X₁₀ is I, X₁₁ is A, and X₁₂ is W. In certain further aspects related to any of the aspects above, X₁₄ is G and X₁₅ is no amino acid or G. In certain further aspects related to any of the aspects above, X₇ is E. In certain further aspects related to any of the aspects above, X₈ is R and X₉ is Q. In certain further aspects related to any of the aspects above, X₁₃ is K. In certain further aspects related to any of the aspects above, X₃ is V. In certain further aspects related to any of the aspects above, X₄ is S, X₅ is Y, and X₆ is M.

The disclosure also provides an isolated polypeptide comprising the amino acid sequence:

(SEQ ID NO: 463)

X₁ X₂ E G X₅ X₆ X₇ S X₉ X₁₀ X₁₁
X₁₂ X₁₃ X₁₄ X₁₅ X₁₆ X₁₇ A I X₂₀ X₂₁ F
X₂₃ X₂₄ X₂₅ L X₂₇ X₂₈ X₂₉ X₃₀;

wherein X₁ is H or Y; X₂ is G or S; X₅ is T or M; X₆ is F or H; X₇ is T or I; X₉ is D or L; X₁₀ is V, F, L, or Y; X₁₁ is S, A, or R; X₁₂ is S, K, A, M, Q or I; X₁₃ is Y, L, H, or A; X₁₄ is M, A, K, I, or L; X₁₅ is E or D; X₁₆ is R, K, or E; X₁₇ is Q or E; X₂₀ is D or E; X₂₁ is E or A; X₂₃ is I, A, or V; X₂₄ is A or N; X₂₅ is W, E, L, F, I, S, N, G, M, H, K or D; X₂₇ is L or A; X₂₈ is A, K, or G; X₂₉ is G, A, or Q; and X₃₀ is no amino acid, G, A, E, or K.

In certain specific aspects, this disclosure provides an AID-related GIP/GLP-1 agonist polypeptide comprising any one or more of the amino acid sequences listed in Table 2.

TABLE 2
GIP/GLP-1 Agonist Peptides based on “AID”

| | SEQ |
|---|---|
| Peptide | ID NO |
| HGEGT FTSDV SSYME RQAID EFIAW LLAG (“AID”) | 33 |
| HGEGT FTSDV SSYME RQAID EFIAW LLKGG | 34 |
| YSEGT FTSDV SSYME RQAID EFIAW LLAG | 35 |
| YSEGT FTSDV SSYME RQAID EFIAW LLKGG | 36 |
| YSEGT FTSDY SSYME RQAID EFIAW LLKGG | 37 |
| YSEGT FTSDV SKLLE RQAID EFIAW LLKGG | 38 |
| YSEGT FTSDV SIAMD RQAID EFIAW LLKGG | 39 |
| YSEGT FTSDV SSYME EEAID EFIAW LLKGG | 40 |
| YSEGT FTSDV SSYME RQAID EFVND LLKGG | 41 |
| YSEGT FTSDV SSYME RQAID EFIAW LLGGG | 42 |
| YSEGT FTSDV SSYME RQAID EFIAW LLAQK | 43 |
| YSEGT FTSDV SSYLE RQAID EFIAW LLKGG | 257 |
| YSEGT FTSDV SSYLE REAID EFIAW LLKGG | 258 |
| YSEGT FTSDV SSYLE RQAIE EFIAW LLKGG | 259 |
| YSEGT FTSDY SKLLE RQAID EFIAW LLKGG | 260 |
| YSEGT FTSDV SSYME RQAID EFIAW LLKQG | 261 |
| YSEGT FISDV SSYLE RQAID EFIAW LLKGG | 262 |
| YSEGT FTSDV SSYME RQAID EFIAW LLKGK | 263 |
| YSEGT FISDV SSYME RQAID EFIAW LLKGG | 264 |
| YSEGT FISDY SSYLE RQAID EFIAW LLKGG | 265 |
| YSEGT FTSDV SSYLE KQAID EFIAW LLKGG | 266 |
| YSEGT FTSDY SKLLE RQAID EFIAE LLKGG | 267 |
| YSEGT FTSDY SKLLE RQAID EFIAL LLKGG | 268 |
| YSEGT FTSDY SKLLE RQAID EFIAD LLKGG | 269 |
| YSEGT FTSDY AAAAE RQAID AFAAW LAAAA | 270 |
| YSEGT FTSDY SKLLE RQAID EFIAF LLKGG | 271 |
| YSEGT FTSDY SKLLE RQAID EFIAI LLKGG | 272 |
| YSEGT FTSDY SKLLE RQAID EFIAS LLKGG | 273 |
| YSEGT FTSDY SKLLE RQAID EFIAN LLKGG | 274 |
| YSEGT FTSDY SKLLE RQAID EFIAG LLKGG | 275 |
| YSEGT FTSDY SKLLE RQAID EFIAM LLKGE | 276 |
| YSEGT FTSDF SKLLE RQAID EFIAW LLKGG | 277 |
| YSEGM HTSDF SKLLE RQAID EFIAW LLKGG | 278 |
| YSEGT FTSDY RMLKE RQAID EFIAW LLKGG | 279 |
| YSEGT FTSDY RILKE RQAID EFIAW LLKGG | 280 |
| YSEGT FTSDY RQAID RQAID EFIAW LLKGG | 281 |
| YSEGT FTSDY SKLLE RQAID EFIAH LLKGG | 282 |
| YSEGT HTSDF SKLLE RQAID EFIAW LLKGG | 283 |
| YSEGT HTSLF SKLLE RQAID EFIAW LLKGG | 284 |
| YSEGT FTSLF SKLLE RQAID EFIAW LLKGG | 285 |
| YSEGT FTSLY RILLE RQAID EFIAW LLKGG | 286 |
| YSEGT FTSDY SIYLD RQAID EFIAW LLKGG | 287 |
| YSEGM HTSDL SKLLE RQAID EFIAW LLKGG | 288 |
| YSEGT FTSDY SKLLE RQAID EFIAH LLKGG | 289 |
| YSEGT FTSDY SKLLE RQAID EFIAW LLKGG | 290 |
| YSEGT HTSDL SKLLE RQAID EFIAW LLKGG | 291 |
| YSEGT FTSLY RIHKE RQAID EFIAK LLKGG | 292 |
| YSEGT HTSDL RILLE RQAID EFIAK LLKGG | 293 |
| YSEGT HTSDL RILLE RQAID EFIAF LLKGG | 294 |

In further aspects, this disclosure provides a GIP/GLP-1 agonist polypeptide comprising a peptide sequence derived from the “AIS” peptide (SEQ ID NO:44). The AIS peptide was selected from a phage display library of peptides based on GLP-1, by binding to GLP-1r and GIPr and confirmed by cAMP selectivity assay. AIS has 9 amino acid substitutions relative to human GLP-1 (1-29).

In certain specific aspects, this disclosure provides an AIS-related GIP/GLP-1 agonist polypeptide comprising any one or more of the amino acid sequences listed in Table 3.

TABLE 3
GIP/GLP-1 Agonist Peptides based on “AIS”

| | SEQ |
|---|---|
| Peptide | ID NO |
| YSEGT FTSDV SSYME RQAIS EFIAW LLAG (“AIS”) | 44 |
| YSEGT FTSDV SSYME RQAIS EFIAW LLKGP | 45 |

In further aspects, this disclosure provides a GIP/GLP-1 agonist polypeptide comprising a peptide sequence derived from the “SIR” peptide (SEQ ID NO:46) or the “KIR” peptide (SEQ ID NO:47). The SIR and KIR peptides were selected from a phage display library of peptides based on GLP-1, by binding to GLP-1r and GIPr and confirmed by cAMP selectivity assay. SIR has 7 amino acid substitutions relative to human GLP-1 (1-29). KIR has 6 amino acid substitutions relative to human GLP-1 (1-29).

In certain specific aspects, this disclosure provides a SIR or KIR-related GIP/GLP-1 agonist polypeptide comprising any one or more of the amino acid sequences listed in Table 4.

TABLE 4
GIP/GLP-1 Agonist Peptides based on “SIR” or “KIR”

| | SEQ |
|---|---|
| Peptide | ID NO |
| HGEGT FTSDV SSYME AQSIR EFIAW LVKGP (“SIR”) | 46 |
| HGEGT FTSDV SSYME AQKID EFIAW LVKG (“KIR”) | 47 |

In further aspects, this disclosure provides a GIP/GLP-1 agonist polypeptide comprising a peptide sequence derived from the “LVR” peptide (SEQ ID NO:48). The LVR peptide was selected from a phage display library of peptides based on GLP-1, by binding to GLP-1r and GIPr and confirmed by cAMP selectivity assay. LVR has 6 amino acid substitutions relative to human GLP-1 (1-29).

In certain specific aspects, this disclosure provides an LVR-related GIP/GLP-1 agonist polypeptide comprising any one or more of the amino acid sequences listed in Table 5.

TABLE 5
GIP/GLP-1 Agonist Peptides based on “LVR”

| | SEQ |
|---|---|
| Peptide | ID NO |
| HGEGT FTSDV SSYME SQLVD EFIAW LVKG | 49 |
| HGEGT FTSDV SSYME SQLVR EFIAW LVKG (“LVR”) | 48 |

In further aspects, this disclosure provides a GIP/GLP-1 agonist polypeptide comprising a peptide sequence derived from GIP or GLP-1 sequences but with amino acids substitutions derived from GIP, GLP-1, and/or exendin-4.

In certain specific aspects, this disclosure provides an GIP/GLP-1 agonist polypeptide comprising any one or more of the amino acid sequences listed in Table 6.

TABLE 6
GIP/GLP-1 Agonist Peptides

| Peptide | SEQ ID NO |
|---|---|
| YSEGT FTSDV SSYLE GQAAK EFIAW LLAG | 50 |
| HGEGT FISDY SIAMD KIHQQ DFVNW LLAQK | 51 |
| HGEGT FTSDV SSYLE GQAAK EFIAW LLAG | 52 |
| YSEGT FTSDV SSYLE EQAAK EFIAW LLAG | 53 |
| YSEGT FTSDV SSYLE GQAAK EFIAW LVAGG | 54 |
| HGEGT FTSDV SSYLE EEAAK EFIAW LVAGG | 55 |
| YSEGT FTSDL SKQME EEAVR LFIEW LKNGG PSSGA PPPS | 56 |
| YSEGT FTSDV SSYLE EEAVR EFIEW LLAGG | 57 |
| YSEGT FISDY SIAMD EEAVR DFVEW LKAGG | 297 |
| YSQGT FTSDY SKILE EEAVR LFIEW LVKGP | 58 |
| HGEGT FTSDV SSYLE GQAAK EFIAW LVKG | 59 |
| HGEGT FTSDV SSYLE EQAAK EFIAW LVKG | 60 |
| HGEGT FTSDV SSYLE EQAAK EFIAW LVKGG | 61 |
| HGEGT FTSDL SKQME EEAVR LFIEW LKNGG PSSGA PPPS | 62 |
| YSEGT FISDY SIAMD KIHQQ DFVNW LLAQK | 63 |
| YSEGT FISDY SIAMD KIHQQ DFVNW LLAQK | 64 |
| YAEGT FISDY SIAMD KIHQQ DFVNW LLAQK GKKND WKHNI TQ | 65 |
| YSEGT FISDY SIALA AAAVR DFVNW LLAGG | 295 |
| YSEGT FISDY SKLLA AAAVR DFVNW LLAGG | 296 |
| YSEGT HTSDL RILLE EEAVR LFIEF LLAGG | 300 |
| YSEGT FISDY SIALA AAAQQ DFVNW LLAGG | 301 |
| YSEGT FISDY SIALA AAAAK DFVNW LLAGG | 302 |
| YSEGT FISDY SIALA AAAID DFVNW LLAGG | 303 |
| YSEGT FTSDY SKLLE AAAAA AFAAW LAAAA | 304 |
| YSEGT FTSDY AAAAE AAAAA AFAAW LAAAA | 305 |
| YSEGT FTSDY SIALA AAAQQ DFVNW LLAGG | 306 |
| YSEGT FISDY SIALA AAAQQ DFVNW LLKGG | 307 |
| YSEGT FTSDY SIALA AAAQQ DFVNW LLKGG | 308 |
| YSEGT FTSDY SAAAE AAAAA AFAAW LAAAA | 309 |
| YSEGT FTSDY AAAAE AAAAA AFAAW LAKAA | 310 |
| YSEGT FISDY SIAMD KIHQQ DFVNW LLAGG | 311 |
| YSEGT FISDY SIAMD KIHQQ DFVNW LLAQK | 312 |
| YSEGT FISDY SIAMD KIHQQ DFVNW LLAQK | 313 |
| YSEGT FISDY SIAMD KIHQQ DFVNW LLAQK | 314 |
| YSEGT FISDY SIAMD KIHQQ DFVNW LLAQK | 315 |
| YSEGT FISDY SIAMD KIHQQ DFVNW LLAQK | 316 |
| YSEGT FISDY SIAMD KIHQQ DFVNW LLAQK PSSGA PPPS | 317 |
| YSEGT FISDY SIAMD KIHQQ DFVNW LLAQK | 318 |
| YSEGT FISDY SIAMD KIHQQ DFVNW LLAQK | 319 |
| YSEGT FISDY SIAMD KIHQQ DFVNW LLAQK | 320 |
| YSEGT FISDY SIAMD KIHQQ DFVNW LLAQK | 321 |
| YSEGT FISDY SIAMD KIHQQ DFVNW LLAQK GAAND WKHNI TQ | 322 |
| YSEGT FISDY SIAMD KIHQQ DFVNW LLAQK GKKND WKHNI TQ | 323 |
| YSEGT FISDY SIAMD KIHQQ DFVNW LLAQK | 324 |
| YSEGT FISDY SIAMD KIHQQ DFVNW LLAQK | 325 |

In certain aspects, any one or more GIP/GLP-1 agonist peptides as described above can be fused to one or more additional heterologous polypeptide domains. Such additional polypeptide regions can facilitate, e.g., activity, efficacy, stability, or in vivo half-life. For example, a heterologous polypeptide domain can comprise a linker, a hinge, an Fc domain, or a combination thereof.

Linkers used in various GIP/GLP-1 agonist polypeptides provided herein can facilitate formation of a desired structure. In some aspects, a polypeptide linker can comprise 1-50 amino acids, 1-25 amino acids, 25-50 amino acids, or 30-50 amino acids. Generally longer linkers correlate with higher activity (more flexible), but also decreased stability as the peptide becomes more exposed. Linkers can comprise, e.g., (Gly-Ser)ₙ, residues, where n is an integer of at least one, and up to, e.g., 4, 5, 6, 10, 20, 50, 100, or more, optionally with some Glu or Lys residues dispersed throughout to increase solubility. Alternatively, certain linkers do not comprise any Serine residues, e.g., where the linker is subject to O-linked glycosylation. In some aspects, linkers can contain cysteine residues, for example, if dimerization of linkers is used to bring two or more GIP/GLP-1 agonist polypeptides into a dimeric configuration. In some aspects, a GIP/GLP-1 agonist polypeptide can comprise at least one, two, three, four, or more linkers. The length and amino acid sequence of a linker can be readily selected and optimized.

In certain aspects, the linker comprises (GGGGS)n, wherein n 1, 2, 3, or 4. For example, certain specific linkers the amino acid sequence: G GGGGS GGGGS GGGGS GGGGS A (SEQ ID NO:66), A PPGGS GGGGS GGGGS GGGGS A (SEQ ID NO:67), GT GGGGS GGGGS GGGGS GGGGS A (SEQ ID NO:68), G GGGGS GGGGS GGGGS GGGGS A (SEQ ID NO:69), G GGGGS A (SEQ ID NO:70), G GGGGS GGGGS A (SEQ ID NO:71), G GGGGS GGGGS GGGGS A (SEQ ID NO:72), G KGGGS GGGGS GGGGS GGGGS A (SEQ ID NO:73), G GGGGS GGGGS GGGGS GGGGSA (SEQ ID NO:179), G GGGG GGGG GGGG GGGG A (SEQ ID NO:180) any combination thereof, any fragment thereof, or any variant thereof. The peptide linkers may optionally contain a potential glycosylation site, for example, but not by way of limitation, a Threonine in SEQ ID NO:68 and the Lysine in SEQ ID NO:73).

In certain aspects, the linker comprises GG SGSTA SSGSG SATGG GGAA (SEQ ID NO:74), any variant thereof, or any fragment thereof. For example, certain specific linkers the amino acid sequence: AAAGG SGSTA SSGSG SATGG GGAA (SEQ ID NO:75), APPGG SGSTA SSGSG SATGG GGAA (SEQ ID NO:76), any combination thereof, any fragment thereof, or any variant thereof.

Hinges used in various GIP/GLP-1 agonist polypeptides provided herein can facilitate formation of a desired structure. In certain aspects, the hinge comprises an amino acid sequence of an IgG1 hinge, an IgG4 hinge, a fragment thereof, a variant thereof, or any combination thereof. In certain, non-limiting aspects, the hinge comprises the amino acid sequence ESKYGPPCPPCPAPEAA (SEQ ID NO:77), THTCPPCPAPEF (SEQ ID NO:78), THTCPPC (SEQ ID NO:79), CPPCPAPEF (SEQ ID NO:80), TYTCPPCPAPEF (SEQ ID NO:81), TSTCPPCPAPEF (SEQ ID NO:82), PPCPPCPAPEF (SEQ ID NO:83), ESKYGPPCPPCPAPEF (SEQ ID NO:84), APEF (SEQ ID NO:85), ESKYGPPCPPC (SEQ ID NO:86), THTCPPCPAPELL (SEQ ID NO:87), any variant thereof, any fragment thereof, or any combination thereof.

Fc regions used in various GIP/GLP-1 agonist polypeptides provided herein can facilitate formation of a desired structure and to enhance or eliminate various desired or undesired effector functions. In certain aspects the Fc region is a native immunoglobulin Fc region.

The terms “Fc domain” and “IgG Fc domain” as used herein refer to the portion of an immunoglobulin, e.g., an IgG molecule that correlates to a crystallizable fragment obtained by papain digestion of an IgG molecule. The Fc region comprises the C-terminal half of two heavy chains of an IgG molecule that are linked by disulfide bonds. For example, an Fc domain can comprise the entire second constant domain CH2 and the third constant domain CH3.

In certain aspects, GIP/GLP-1 agonist polypeptides provided herein can comprise a “wild type IgG Fc domain,” e.g., any naturally occurring IgG Fc region (any allele). In certain aspects the IgG Fc domain is an IgG1 domain (SEQ ID NO:181), in some aspects the IgG Fc domain is an IgG4 Fc domain (SEQ ID NO:182).

In certain aspects, GIP/GLP-1 agonist polypeptides provided herein can comprise a “variant IgG Fc domain,” an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an FcγR and/or C1q as compared to the wild type Fc domain not comprising the one or more amino acid substitutions.

In certain aspects, GIP/GLP-1 agonist polypeptides provided herein can comprise a “TM” Fc domain. The terms “TM” or “TM mutant” refer to a set of mutations in an IgG Fc domain that result in ablation of effector function, namely elimination of the Fc domain's ability to mediate antibody-dependent cell-mediated cytotoxicity and complement-mediated cytotoxicity. In certain aspects, a TM mutant can comprise a combination of three “TM mutations”: L234F, L235E, and P331S, where the numbering is according to the EU index as in Kabat. These mutations cause a profound decrease Fc domain binding to human FcγRI (CD64), FcγRIIA (CD32A), FcγRIII (CD16) and C1q. See, e.g., US 2011/0059078 and Oganesyan et al. Acta Crystallographica D 64:700-704 (2008), which are hereby incorporated by reference in their entireties. A human IgG1 Fc domain comprising the TM mutations is shown as SEQ ID NO:183.

In certain aspects, GIP/GLP-1 agonist polypeptides provided herein can comprise a “YTE” Fc domain. The terms “YTE” or “YTE mutant” refer to a set of mutations in an IgG1 Fc domain that results in an increase in the binding to human FcRn and improves the serum half-life of the antibody having the mutation. A YTE mutant comprises a combination of three “YTE mutations”: M252Y, S254T, and T256E, wherein the numbering is according to the EU index as in Kabat, introduced into the heavy chain of an IgG. See U.S. Pat. No. 7,658,921, which is incorporated by reference herein. The YTE mutant has been shown to increase the serum half-life of antibodies compared to wild-type versions of the same antibody. See, e.g., Dall'Acqua et al., J. Biol. Chem. 281:23514-24 (2006) and U.S. Pat. No. 7,083,784, which are hereby incorporated by reference in their entireties. A human IgG1 Fc domain comprising the YTE mutations is shown as SEQ ID NO:184.

In certain aspects, GIP/GLP-1 agonist polypeptides provided herein can comprise a “TM-YTE IgG Fc domain.” The term “TM-YTE IgG Fc domain” refers to an IgG Fc domain comprising one or more of the three “TM” mutations (L234F/L235E/P331S) and one or more of the three “YTE” mutations (M252Y/S254T/T256E), where all the numbering is according to the EU index as in Kabat. A human IgG1 Fc domain comprising the TM mutations and the YTE mutations is shown as SEQ ID NO:186.

In certain aspects, GIP/GLP-1 agonist polypeptides provided herein can comprise an Fc domain with additional mutations to provide additional stability. In some aspects the variant IgG Fc domain can comprise, alone or in addition to YTE and/or TM mutations one or more of the following mutations:
(i) a phenylalanine (F) amino acid at EU position 234,
(ii) an alanine (A), asparagine (N), phenylalanine (F), a glutamine (Q) or a valine (V) amino acid at EU position 235, and
(iii) an alanine (A), aspartic acid (D), glutamic acid (E), histidine (H), asparagine (N), or glutamine (Q) amino acid at EU position 322, or—in the alternative—an alanine (A) or glycine (G) amino acid at EU position 331.

In certain aspects, GIP/GLP-1 agonist polypeptides provided herein can comprise an “FQQ” mutation: an IgG1 Fc domain with a phenylalanine (F) amino acid at EU position 234, a glutamine (Q) amino acid at EU position 235, and a glutamine (Q) amino acid at EU position 322. The FQQ mutation can be in combination with a YTE mutation, an TM mutation, or both a YTE mutation and a TM mutation. A human IgG1 Fc domain comprising the FQQ mutations is shown as SEQ ID NO:185.

In sum, a GIP/GLP-1 agonist polypeptide as provided herein can comprise, without limitation, an Fc domain, e.g., an IgG1 Fc domain, an IgTM Fc domain, IgG1-FQQ Fc domain, an IgG4 Fc domain, a YTE Fc domain, any fragment thereof, any variant thereof, or any combination thereof.

In certain specific aspects, a GIP/GLP-1 agonist polypeptide comprising a linker, a hinge, and an Fc domain can comprise one or more of the G89 polypeptides provided in Table 7.

TABLE 7
G89-based GIP/GLP-1 Agonist Polypeptides

| | | | | | SEQ ID |
|---|---|---|---|---|---|
| ID | IgG Fc | Agonist Peptide | Linker | Hinge | NO |
| IP024 | IgG1-TM | YSEGT FTSDY SKLLE | AAAGG SGSTA | THTCPPC | 88 |
| | | EEAVR LFIEW LLAG | SSGSG SATGG | PAPEF | |
| | | | GGAA | | |
| IP085 | IgG1-TM | YSEGT FTSDY SKLLE | APPGG SGSTA | THTCPPC | 89 |
| | | EEAVR LFIEW LLAG | SSGSG SATGG | PAPEF | |
| | | | GGAA | | |
| IP131 | IgG1-TM | YSEGT FTSDY SKLLE | G GGGGS GGGGS | THTCPPC | 90 |
| | | EEAVR LFIEW LLAG | GGGGS GGGGS A | PAPEF | |
| IP266 | IgG1-TM | YSEGT FTSDY SKLLE | A PPGGS GGGGS | THTCPPC | 91 |
| | | EEAVR LFIEW LLAG | GGGGS GGGGS A | PAPEF | |
| IP229 | IgG1- | YSEGT FTSDY SKLLE | AAAGG SGSTA | THTCPPC | 92 |
| | FQQ | EEAVR LFIEW LLAG | SSGSG SATGG | PAPEF | |
| | | | GGAA | | |
| IP244 | IgG1-TM | YSEGT FTSDV SKLLE | APPGG SGSTA | THTCPPC | 93 |
| | | EEAVR LFIEW LLAG | SSGSG SATGG | PAPEF | |
| | | | GGAA | | |
| IP253 | IgG1-TM | YSEGT FTSDY | APPGG SGSTA | THTCPPC | 94 |
| | | SKLLD EEAVR | SSGSG SATGG | PAPEF | |
| | | LFIEW LLAG | GGAA | | |
| IP124 | IgG1-TM | YSEGT FTSDY SKLLE | AAAGG SGSTA | THTCPPC | 95 |
| | | EEAVR LFIEW LVKG | SSGSG SATGG | PAPEF | |
| | | | GGAA | | |
| IP254 | IgG1-TM | YSEGT FTSDY SKLLE | GT GGGGS GGGGS | THTCPPC | 96 |
| | | EEAVR LFIEW | GGGGS GGGGS A | PAPEF | |
| | | LLAGN | | | |
| IP256 | IgG1-TM | YSEGT FTSDY SKLLE | G GGGGS GGGGS | THTCPPC | 97 |
| | | EEAVR LFIEW | GGGGS GGGGS A | PAPEF | |
| | | LLAGG HNITQ | | | |
| IP213 | IgG1-TM | YSEGT FTSDY SKLLE | AAAGG SGSTA | THTCPPC | 98 |
| | | EEAVR LFIEW | SSGSG SATGG | PAPEF | |
| | | LLAGG | GGAA | | |
| IP087 | IgG1-TM | YSEGT FTSDY SKLLE | AAAGG SGSTA | THTCPPC | 99 |
| | | EEAVR LFIEW | SSGSG SATGG | PAPEF | |
| | | LLAGP | GGAA | | |
| IP201 | IgG1-TM | YSEGT FTSDY SKLLE | AAAGG SGSTA | THTCPPC | 100 |
| | | EEAVR LFIEW | SSGSG SATGG | PAPEF | |
| | | LLAGP SSGAP PPS | GGAA | | |
| IP249 | IgG1-TM | YSEGT FTSDY SKLLE | AAAGG SGSTA | THTCPPC | 101 |
| | | EEAVR LFIEW | SSGSG SATGG | PAPEF | |
| | | LLAQK | GGAA | | |
| IP228 | IgG1-TM | YSEGT FTSDY SKLLE | AAAGG SGSTA | THTCPPC | 102 |
| | | EEAVR LFIEW | SSGSG SATGG | PAPEF | |
| | | LKNGG | GGAA | | |
| IP222 | IgG1-TM | YSEGT FTSDY SKLLE | AAAGG SGSTA | THTCPPC | 103 |
| | | EEAVR LFIEW | SSGSG SATGG | PAPEF | |
| | | LKNGG PSSGA PPPS | GGAA | | |
| IP255 | IgG1-TM | YSEGT FTSDY SKLLE | G GGGGS GGGGS | THTCPPC | 104 |
| | | EEAVR LFIEW | GGGGS GGGGS A | PAPEF | |
| | | LKNGT | | | |
| IP088 | IgG1-TM | YSEGT FTSDY SKLLE | AAAGG SGSTA | THTCPPC | 105 |
| | | EEAVR LFIEW | SSGSG SATGG | PAPEF | |
| | | LVKGP | GGAA | | |
| IP147 | IgG1-TM | YSEGT FTSDY SKLLE | APPGG SGSTA | THTCPPC | 106 |
| | | EEAVR LFIEW | SSGSG SATGG | PAPEF | |
| | | LVKGP | GGAA | | |
| IP149 | IgG1-TM | YSQGT FTSDY | APPGG SGSTA | THTCPPC | 107 |
| | | SKLLE EEAVR LFIEW | SSGSG SATGG | PAPEF | |
| | | LVKGP | GGAA | | |
| IP234 | IgG1-TM | YSEGT FTSDY SKLLE | G GGGGS A | THTCPPC | 108 |
| | | EEAVR LFIEW | | PAPEF | |
| | | LVKGP | | | |
| IP235 | IgG1-TM | YSEGT FTSDY SKLLE | G GGGGS GGGGSA | THTCPPC | 109 |
| | | EEAVR LFIEW | | PAPEF | |
| | | LVKGP | | | |
| IP236 | IgG1-TM | YSEGT FTSDY SKLLE | G GGGGS GGGGS | THTCPPC | 110 |
| | | EEAVR LFIEW | GGGGS A | PAPEF | |
| | | LVKGP | | | |
| IP237 | IgG1-TM | YSEGT FTSDY SKLLE | G GGGGS GGGGS | THTCPPC | 111 |
| | | EEAVR LFIEW | GGGGS GGGGS A | PAPEF | |
| | | LVKGP | | | |
| IP239 | IgG1-TM | YSEGT FTSDY SKLLE | AAAGG SGSTA | CPPCPAP | 112 |
| | | EEAVR LFIEW | SSGSG SATGG | EF | |
| | | LVKGP | GGAA | | |
| IP210 | IgG1-TM | YSEGT FTSDL SKLLE | AAAGG SGSTA | THTCPPC | 113 |
| | | EEAVR LFIEW | SSGSG SATGG | PAPEF | |
| | | LVKGP | GGAA | | |
| IP211 | IgG1-TM | YSEGT FTSDY | AAAGG SGSTA | THTCPPC | 114 |
| | | SSYME EEAVR | SSGSG SATGG | PAPEF | |
| | | LFIEW LVKGP | GGAA | | |
| IP245 | IgG1-TM | YSEGT FTSDY | AAAGG SGSTA | THTCPPC | 115 |
| | | SIAMD EEAVR | SSGSG SATGG | PAPEF | |
| | | LFIEW LVKGP | GGAA | | |
| IP246 | IgG1-TM | YSEGT FTSDY SKLLE | AAAGG SGSTA | THTCPPC | 116 |
| | | GQAAK LFIEW | SSGSG SATGG | PAPEF | |
| | | LVKGP | GGAA | | |
| IP247 | IgG1-TM | YSEGT FTSDY SKLLE | AAAGG SGSTA | THTCPPC | 117 |
| | | KIHQQ LFIEW | SSGSG SATGG | PAPEF | |
| | | LVKGP | GGAA | | |
| IP243 | IgG1-TM | YSEGT FTSDY SKLLE | AAAGG SGSTA | THTCPPC | 118 |
| | | EEAQR LFIEW | SSGSG SATGG | PAPEF | |
| | | LVKGP | GGAA | | |
| IP212 | IgG1-TM | YSEGT FTSDY SKLLE | AAAGG SGSTA | THTCPPC | 119 |
| | | EEAVR EFIAW | SSGSG SATGG | PAPEF | |
| | | LVKGP | GGAA | | |
| IP248 | IgG1-TM | YSEGT FTSDY SKLLE | AAAGG SGSTA | THTCPPC | 120 |
| | | EEAVR DFVNW | SSGSG SATGG | PAPEF | |
| | | LVKGP | GGAA | | |
| IP252 | IgG4 | YSEGT FTSDY SKLLE | G GGGGS GGGGS | ESKYGPP | 121 |
| | | EEAVR LFIEW | GGGGS A | CPPCPAP | |
| | | LVKGP | | EAA | |
| IP257 | IgG1-TM | _SEGT FTSDY SKLLE | G GGGGS GGGGS | THTCPPC | 122 |
| | | EEAVR LFIEW | GGGGS GGGGS A | PAPEF | |
| | | LVKGP | | | |
| IP258 | IgG1-TM | YSEGT FTSDY | G GGGGS GGGGS | THTCPPC | 123 |
| | | SKLLD EEAVR | GGGGS GGGGS A | PAPEF | |
| | | LFIEW LVKGP | | | |
| IP259 | IgG1-TM | YSEGT FTSDY SKLLE | G GGGGS GGGGS | THTCPPC | 124 |
| | | KEAVR LFIEW | GGGGS GGGGS A | PAPEF | |
| | | LVKGP | | | |
| IP260 | IgG1-TM | YSEGT FTSDY SKLLE | G GGGGS GGGGS | THTCPPC | 125 |
| | | EEAVQ LFIEW | GGGGS GGGGS A | PAPEF | |
| | | LVKGP | | | |
| IP261 | IgG1-TM | YSEGT FTSDY SKLLE | G GGGGS GGGGS | THTCPPC | 126 |
| | | EEAVR LFVEW | GGGGS GGGGS A | PAPEF | |
| | | LVKGP | | | |
| IP262 | IgG1-TM | YSEGT FTSDY SKLLE | G GGGGS GGGGS | THTCPPC | 127 |
| | | EEAVR LFIEW | GGGGS GGGGS A | PAPEF | |
| | | LLKGP | | | |
| IP263 | IgG1-TM | YSEGT FTSDY SKLLE | G GGGGS GGGGS | THTCPPC | 128 |
| | | EEAVR LFIEW | GGGGS GGGGS A | PAPEF | |
| | | LVKQP | | | |
| IP264 | IgG1-TM | YSEGT FTSDY SKLLE | G GGGGS GGGGS | THTCPPC | 129 |
| | | EEAVR LFIEW | GGGGS GGGGS A | PAPEF | |
| | | LVKGK | | | |
| IP265 | IgG1-TM | YSEGT FTSDY SKLLE | G KGGGS GGGGS | THTCPPC | 130 |
| | | EEAVR LFIEW | GGGGS GGGGS A | PAPEF | |
| | | LVKGP | | | |
| IP266 | IgG1-TM | YSEGT FTSDY SKLLE | A PPGGS GGGGS | THTCPPC | 326 |
| | | EEAVR LFIEW LLAG | GGGGS GGGGS A | PAPEF | |
| IP267 | IgG1-TM | YSEGT FTSDY SKLLE | G GGGGS GGGGS | THTCPPC | 327 |
| | | EEHVR LFIEW | GGGGS GGGGS A | | |
| | | LVKGP | | | |
| IP268 | IgG1- | YSEGT FTSDY SKLLE | G GGGGS GGGGS | THTCPPC | 328 |
| | TM(ΔK) | EEAVR LFIEW | GGGGS GGGGS A | | |
| | | LVKGP | | | |
| IP272 | IgG1-TM | YSEGT FTSDY SKLLE | APPGG SGSTA | THTCPPC | 329 |
| | | EEAVR LFIEW | SSGSG SATGG | | |
| | | LLAQK | GGAA | | |
| IP273 | IgG1- | YSEGT FTSDY SKLLE | AAAGG SGSTA | THTCPPC | 330 |
| | TM(ΔK) | EEAVR LFIEW | SSGSG SATGG | | |
| | | LVKGP | GGAA | | |
| IP274 | IgG1-TM | YSEGT FTSDY SKLLE | G GGGGS GGGGS | THTCPPC | 331 |
| | | KEAVR LFIEW | GGGGS GGGGS A | | |
| | | LLKGP | | | |
| IP275 | IgG1-TM | YSEGT FTSDY SKLLE | G GGGGS GGGGS | THTCPPC | 332 |
| | | KEAQR LFIEW | GGGGS GGGGS A | | |
| | | LLKGP | | | |
| IP276 | IgG1-TM | YSEGT FTSDY SKLLE | G GGGG GGGG | THTCPPC | 333 |
| | | EEAVR LFIEW | GGGG GGGG A | | |
| | | LVKGP | | | |
| IP278 | IgG1- | YSEGT FTSDY SKLLE | G GGGGS GGGGS | THTCPPC | 334 |
| | TM(ΔK) | EEAVR LFIEW | GGGGS GGGGS A | | |
| | | LVKGP PSSGA PPPS | | | |
| IP279 | IgG1- | YSEGT FTSDY SKLLE | G GGGGS GGGGS | THTCPPC | 335 |
| | TM(ΔK) | EEAVR LFIEW | GGGGS GGGGS A | | |
| | | LVKGP SSGAP PPS | | | |
| P280 | IgG1- | YSEGT FTSDY SKLLE | G GGGGS GGGGS | THTCPPC | 336 |
| | TM(ΔK) | EEAVR LFIEW | GGGGS GGGGS A | | |
| | | LVKGG PSSGA PPPS | | | |
| IP281 | IgG1- | YSEGT FTSDY SKLLE | G GGGGS GGGGS | CPPC | 337 |
| | TM(ΔK) | EEAVR LFIEW | GGGGS GGGGS A | | |
| | | LVKGP | | | |
| IP283 | IgG1-TM | YSEGT FTSDY SKLLE | GGGGG GGA | THTCPPC | 338 |
| | | EEAVR LFIEW | | | |
| | | LVKGP | | | |
| IP284 | IgG1-TM | YSEGT FTSDY SKLLE | GGGGG GGGGG | THTCPPC | 339 |
| | | EEAVR LFIEW | GGA | | |
| | | LVKGP | | | |
| IP285 | IgG1-TM | YSEGT FTSDY SKLLE | GGGGG GGGGG | THTCPPC | 340 |
| | | EEAVR LFIEW | GGGGG GGGGG | | |
| | | LVKGP | GGGA | | |
| IP286 | IgG1-TM | YSEGT FTSDY SKLLE | GGGGG GGGGG | THTCPPC | 341 |
| | | EEAVR LFIEW | GGGGG GGGGG | | |
| | | LVKGP | GGGGG GGA | | |
| IP288 | IgG1- | Negative Control | G GGGGS GGGGS | THTCPPC | n/a |
| | TM(ΔK) | | GGGGS GGGGS A | | |
| IP289 | IgG1- | YSEGT FTSDY SKLLE | G GGGGS GGGGS | THTCPPC | 342 |
| | TM(ΔK) | KEAER LFIEW | GGGGS GGGGS A | | |
| | | LLKGP | | | |
| IP290 | IgG1- | YSEGT FTSDY SKLLE | GGGGG GGGGG | CPPC | 343 |
| | TM(ΔK) | EEAVR LFIEW | GGGGG GGGGG A | | |
| | | LVKGP | | | |
| IP291 | IgG1- | YSEGT FTSDY SKLLE | GGGGG GGGGG | CPPC | 344 |
| | TM(ΔK) | EECVR LFIEW | GGGGG GGA | | |
| | | LVKGG PGGGA | | | |
| | | PPPGC | | | |
| IP292 | IgG1- | YSEGT FTSDY SKLLE | GGGGG GGGGG | CPPC | 345 |
| | TM(ΔK) | EEAVR CFIEW | GGGGG GGA | | |
| | | LVKGG PGGGA | | | |
| | | PPCGG | | | |
| IP293 | IgG1- | YSEGT FTSDY SKLLE | GGGGG GGGGG | CPPC | 346 |
| | TM(ΔK) | EEAVR CFIEC LVKGP | GGGGG GGGGG A | | |
| IP294 | IgG1- | YSEGT FTSDY SKLLE | GGGGG GGGGG | CPPC | 347 |
| | TM(ΔK) | EEAVR LFICW | GGGGG GGGGG A | | |
| | | LVCGP | | | |
| IP295 | IgG1- | YSEGT FTSDY SKLLE | G GGGGS GGGGS | THTCPPC | 348 |
| | TM(ΔK) | EEAVR LFIEW | GGGGS GGGGS A | | |
| | | LLAGG PSSGA PPPS | | | |
| IP296 | IgG1- | YSEGT FTSDY SKLLE | GGGGG GGGGG | CPPC | 349 |
| | TM(ΔK) | EEAVR CFIEW | GGGGG GGGGG | | |
| | | LVKGG PSSGA PPCS | GGGGA | | |
| IP297 | IgG1- | YSEGT FTSDY SKLLE | GGGGG GGGGG | CPPC | 350 |
| | TM(ΔK) | EEAVR LFIEW LLAG | GGGGG GGGGG | | |
| | | | GGGGA | | |
| IP299 | IgG1- | YSEGT FISDY SKLLE | G GGGGS GGGGS | THTCPPC | 351 |
| | TM(ΔK) | EEAVR DFVEW | GGGGS GGGGS A | | |
| | | LKAGG | | | |
| IP300 | IgG1- | YSEGT FTSDY SKLLE | APPGG GGGGG | CPPC | 352 |
| | TM(ΔK) | EEAVR LFIEW LLAG | GGGGG GGGGG | | |
| | | | GGGGA | | |
| IP322 | IgG1-TM | YSEGT FTSDY SKLLE | G GGGGS GGGGS | no hinge | 353 |
| | | EEAVR LFIEW | GGGGS GGGGS | | |
| | | LVKGP | GGGGS A | | |
| IP323 | IgG1-TM | YSEGT FTSDY SKLLE | G GGGGS GGGGS | no hinge | 354 |
| | | EEAVR LFIEW | GGGGS GGGGS | | |
| | | LVKGP | GGGGS GGGGS A | | |
| IP324 | IgG1-TM | YSEGT FTSDY SKLLE | G GGGGS GGGGS | no hinge | 355 |
| | | EEAVR LFIEW | GGGGS GGGGS | | |
| | | LVKGP | GGGGS GGGGS | | |
| | | | GGGGS A | | |
| IP325 | IgG1-TM | YSEGT FTSDY SKLLE | G GGGGS GGGGS | no hinge | 356 |
| | | EEAVR LFIEW | GGGGS GGGGS | | |
| | | LVKGP | GGGGS GGGGS | | |
| | | | GGGGS GGGGS A | | |
| IP326 | IgG1-TM | YSEGT FTSDY SKLLE | G GGGGS GGGGS | no hinge | 357 |
| | | EEAVR LFIEW | GGGGS GGGGS | | |
| | | LLAGG | GGGGS A | | |
| IP327 | IgG1-TM | YSEGT FTSDY SKLLE | G GGGGS GGGGS | no hinge | 358 |
| | | EEAVR LFIEW | GGGGS GGGGS | | |
| | | LLAGG | GGGGS GGGGS A | | |
| IP328 | IgG1-TM | YSEGT FTSDY SKLLE | G GGGGS GGGGS | no hinge | 359 |
| | | EEAVR LFIEW | GGGGS GGGGS | | |
| | | LLAGG | GGGGS GGGGS | | |
| | | | GGGGS GGGGS A | | |
| IP339 | IgG1-TM | YSEGT FTSDY SKLLE | G GGGGS GGGGS | no hinge | 360 |
| | | KEAQR LFIEW | GGGGS GGGGS | | |
| | | LLAGG | GGGGS GGGGS A | | |
| IP341 | IgG1-TM | HGEGT FTSDL | GS GGGGS GGGGS | no hinge | 361 |
| | | SKQME EEAVR | GGGGS GGGGS | | |
| | | LFIEW LKNGG | GGGGS A | | |
| | | PSSGA PPPS | | | |
| IP354 | IgG1-TM | YSEGM HTSDF | GGGGS GGGGS | THTCPPC | 362 |
| | | SKLLE EEAVR LFIEW | GGGGS GGGGS A | | |
| | | LLAGG | | | |
| IP355 | IgG1-TM | YSEGT HTSLF SKLLE | GGGGS GGGGS | THTCPPC | 363 |
| | | EEAVR LFIEW | GGGGS GGGGS A | | |
| | | LLAGG | | | |
| IP357 | IgG1-TM | YSEGT FTSDF SKLLE | GGGGS GGGGS | THTCPPC | 364 |
| | | EEAVR LFIEF LLAGG | GGGGS GGGGS A | | |
| IP358 | IgG1-TM | YSEGT FTSDF SKLLE | GGGGS GGGGS | THTCPPC | 365 |
| | | EEAVR LFIEW | GGGGS GGGGS A | | |
| | | LLAGG | | | |
| IP359 | IgG1-TM | YSEGT FTSDY SKLLE | GGGGS GGGGS | THTCPPC | 366 |
| | | EEAVR LFIEH | GGGGS GGGGS A | | |
| | | LLAGG | | | |
| IP360 | IgG1-TM | YSEGT FTSDY RILLE | GGGGS GGGGS | THTCPPC | 367 |
| | | EEAVR LFIEW | GGGGS GGGGS A | | |
| | | LLAGG | | | |
| IP361 | IgG1-TM | YSEGT FTSDY RILKE | GGGGS GGGGS | THTCPPC | 368 |
| | | EEAVR LFIEW | GGGGS GGGGS A | | |
| | | LLAGG | | | |
| IP362 | IgG1-TM | YSEGT FTSDY | GGGGS GGGGS | THTCPPC | 369 |
| | | MMLKE EEAVR | GGGGS GGGGS A | | |
| | | LFIEW LLAGG | | | |
| IP363 | IgG1-TM | YSEGT FTSDY | GGGGS GGGGS | THTCPPC | 370 |
| | | RMLKE EEAVR | GGGGS GGGGS A | | |
| | | LFIEW LLAGG | | | |
| IP370 | IgG1- | YSEGT FTSLY SKLKE | GGGGS GGGGS | THTCPPC | 371 |
| | fusion | EEAVR LFIEW | GGGGS GGGGS A | | |
| | | LLAGG | | | |
| IP371 | IgG1- | YSEGM HTSDL | G GGGGS GGGGS | CPPC | 372 |
| | fusion | SKLLE EEAVR LFIEW | GGGGS GGGGS A | | |
| | | LLAGG | | | |
| IP372 | IgG1- | YSEGT FTSDY SIYLD | G GGGGS GGGGS | CPPC | 373 |
| | TM(ΔK) | EEAVR LFIEW | GGGGS GGGGS A | | |
| | | LLAGG | | | |
| IP374 | IgG1- | YSEGT FTSLY SKLLE | GGGGS GGGGS | THTCPPC | 374 |
| | fusion | EEAVR LFIEW | GGGGS GGGGS A | | |
| | | LLAGG | | | |
| IP379 | IgG1- | YSEGT HTSDF SKLLE | GGGGS GGGGS | THTCPPC | 375 |
| | fusion | EEAVR LFIEW | GGGGS GGGGS A | | |
| | | LLAGG | | | |
| IP380 | IgG1- | YSEGT FTSDY SKLLE | GGGGS GGGGS | THTCPPC | 376 |
| | fusion | EEAVR LFIEF LLAGG | GGGGS GGGGS A | | |
| IP381 | IgG1- | YSEGT FTSDY SKLLE | GGGGS GGGGS | THTCPPC | 377 |
| | fusion | EEAVR LFIEY | GGGGS GGGGS A | | |
| | | LLAGG | | | |
| IP382 | IgG1- | YSEGT FTSLY SKLLE | GGGGS GGGGS | THTCPPC | 378 |
| | fusion | EEAVR LFIEY | GGGGS GGGGS A | | |
| | | LLAGG | | | |
| IP383 | IgG1-TM | YSEGT FTSLY RILLE | GGGGS GGGGS | THTCPPC | 379 |
| | | EEAVR LFIEW | GGGGS GGGGS A | | |
| | | LLAGG | | | |
| IP384 | IgG1- | YSEGT FTSDY SKLLE | GGGGS GGGGS | THTCPPC | 380 |
| | fusion | EEAVR LFIEW | GGGGS GGGGS A | | |
| | | LLKGG | | | |
| IP390 | IgG1-TM | YSEGT FTSDY SKLLE | G GGGGS GGGGS | CPPC | 381 |
| | | EEAVR LFIEW | GGGGS GGGGS | | |
| | | LVKGP | GGGGS A | | |
| IP400 | IgG1-TM | YSEGT HTSDL | GGGGS GGGGS | THTCPPC | 382 |
| | | SKLLE EEAVR LFIEW | GGGGS GGGGS A | | |
| | | LLAGG | | | |
| IP403 | IgG1- | YSEGT HTSDL RILLE | GS GGGGS GGGGS | CPPC | 383 |
| | TM(ΔK) | EEAVR LFIEK | GGGGS GGGGS | | |
| | | LLAGG | GGGGS GGGGS | | |
| | | | GGGGS A | | |
| IP407 | IgG1-TM | YSEGT FTSDY SKLLE | GGGGS GGGGS | THTCPPC | 384 |
| | | EEAIR LFIEW LLAGG | GGGGS GGGGS A | | |
| IP410 | IgG1- | YSEGT FTSLY RIHKE | GS GGGGS GGGGS | CPPC | 385 |
| | TM(ΔK) | EEAVR LFIEK | GGGGS GGGGS | | |
| | | LLAGG | GGGGS GGGGS | | |
| | | | GGGGS A | | |
| IP411 | IgG1- | YGEGT FTSDY | GGGGG GGGGG | CPPC | 386 |
| | TM(ΔK) | SKLLE EEAVR LFIEW | GGGGG GGGGG A | | |
| | | LVKGP | | | |
| IP412 | IgG1-TM | YSEGT FTSDY SKLLE | GGGGS GGGGS | THTCPPC | 387 |
| | | EEANR LFIEW | GGGGS GGGGS A | | |
| | | LLAGG | | | |
| IP413 | IgG1-TM | YSEGT FTSDY SKLLE | GGGGS GGGGS | THTCPPC | 388 |
| | | EEASR LFIEW | GGGGS GGGGS A | | |
| | | LLAGG | | | |
| IP415 | IgG1- | HSQGT FTSDY SKILE | GS GGGGS GGGGS | CPPC | 389 |
| | TM(ΔK) | EEAVR LFIEW | GGGGS GGGGS | | |
| | | LLAGG | GGGGS GGGGS | | |
| | | | GGGGS A | | |
| IP416 | IgG1- | YSEGT FTSDY SKLLE | GS GGGGS GGGGS | CPPC | 390 |
| | TM(ΔK) | EEAVR LFIEW | GGGGS GGGGS | | |
| | | LLAGG | GGGGS GGGGS | | |
| | | | GGGGS A | | |
| IP420 | IgG1-TM | YSEGT FTSDY SKLLE | GGGGS GGGGS | THTCPPC | 391 |
| | | EEATR LFIEW | GGGGS GGGGS A | | |
| | | LLAGG | | | |

In certain specific aspects, a GIP/GLP-1 agonist polypeptide comprising a linker, a hinge, and an Fc domain can comprise one or more of the AID-based polypeptides provided in Table 8.

TABLE 8
AID-based GIP/GLP-1 Agonist Polypeptides

| | | | | | SEQ ID |
|---|---|---|---|---|---|
| ID | IgG Fc | Agonist Peptide | Linker | Hinge | NO |
| IP172 | IgG1-TM | HGEGT FTSDV | AAAGG SGSTA | THTCPPC | 131 |
| | | SSYME RQAID | SSGSG SATGG | PAPEF | |
| | | EFIAW LLAG | GGAA | | |
| IP198 | IgG1-TM | HGEGT FTSDV | AAAGG SGSTA | THTCPPC | 132 |
| | | SSYME RQAID | SSGSG SATGG | PAPEF | |
| | | EFIAW LLKGG | GGAA | | |
| IP197 | IgG1-TM | YSEGT FTSDV | AAAGG SGSTA | THTCPPC | 133 |
| | | SSYME RQAID | SSGSG SATGG | PAPEF | |
| | | EFIAW LLAG | GGAA | | |
| IP170 | IgG1-TM | YSEGT FTSDV | AAAGG SGSTA | THTCPPC | 134 |
| | | SSYME RQAID | SSGSG SATGG | PAPEF | |
| | | EFIAW LLKGG | GGAA | | |
| IP209 | IgG1-TM | YSEGT FTSDY | AAAGG SGSTA | THTCPPC | 135 |
| | | SSYME RQAID | SSGSG SATGG | PAPEF | |
| | | EFIAW LLKGG | GGAA | | |
| IP226 | IgG1-TM | YSEGT FTSDV SKLLE | AAAGG SGSTA | THTCPPC | 136 |
| | | RQAID EFIAW | SSGSG SATGG | PAPEF | |
| | | LLKGG | GGAA | | |
| IP250 | IgG1-TM | YSEGT FTSDV | AAAGG SGSTA | THTCPPC | 137 |
| | | SIAMD RQAID | SSGSG SATGG | PAPEF | |
| | | EFIAW LLKGG | GGAA | | |
| IP223 | IgG1-TM | YSEGT FTSDV | AAAGG SGSTA | THTCPPC | 138 |
| | | SSYME EEAID EFIAW | SSGSG SATGG | PAPEF | |
| | | LLKGG | GGAA | | |
| IP227 | IgG1-TM | YSEGT FTSDV | AAAGG SGSTA | THTCPPC | 139 |
| | | SSYME RQAID | SSGSG SATGG | PAPEF | |
| | | EFVND LLKGG | GGAA | | |
| IP224 | IgG1-TM | YSEGT FTSDV | AAAGG SGSTA | THTCPPC | 140 |
| | | SSYME RQAID | SSGSG SATGG | PAPEF | |
| | | EFIAW LLGGG | GGAA | | |
| IP251 | IgG1-TM | YSEGT FTSDV | AAAGG SGSTA | THTCPPC | 141 |
| | | SSYME RQAID | SSGSG SATGG | PAPEF | |
| | | EFIAW LLAQK | GGAA | | |
| IP298 | IgG1-TM | YSEGT FTSDV SSYLE | AAAGG SGSTA | THTCPPC | 392 |
| | | RQAID EFIAW | SSGSG SATGG | | |
| | | LLKGG | GGAA | | |
| IP301 | IgG1-TM | YSEGT FTSDV SSYLE | AAAGG SGSTA | THTCPPC | 393 |
| | | REAID EFIAW | SSGSG SATGG | | |
| | | LLKGG | GGAA | | |
| IP304 | IgG1-TM | YSEGT FTSDV SSYLE | AAAGG SGSTA | THTCPPC | 394 |
| | | RQAIE EFIAW | SSGSG SATGG | | |
| | | LLKGG | GGAA | | |
| IP306 | IgG1-TM | YSEGT FTSDY SKLLE | AAAGG SGSTA | THTCPPC | 395 |
| | | RQAID EFIAW | SSGSG SATGG | | |
| | | LLKGG | GGAA | | |
| IP307 | IgG1-TM | YSEGT FTSDV | AAAGG SGSTA | THTCPPC | 396 |
| | | SSYME RQAID | SSGSG SATGG | | |
| | | EFIAW LLKQG | GGAA | | |
| IP309 | IgG1-TM | YSEGT FISDV SSYLE | AAAGG SGSTA | THTCPPC | 397 |
| | | RQAID EFIAW | SSGSG SATGG | | |
| | | LLKGG | GGAA | | |
| IP310 | IgG1-TM | YSEGT FTSDV | AAAGG SGSTA | THTCPPC | 398 |
| | | SSYME RQAID | SSGSG SATGG | | |
| | | EFIAW LLKGK | GGAA | | |
| IP312 | IgG1-TM | YSEGT FISDV SSYME | AAAGG SGSTA | THTCPPC | 399 |
| | | RQAID EFIAW | SSGSG SATGG | | |
| | | LLKGG | GGAA | | |
| IP315 | IgG1-TM | YSEGT FISDY SSYLE | AAAGG SGSTA | THTCPPC | 400 |
| | | RQAID EFIAW | SSGSG SATGG | | |
| | | LLKGG | GGAA | | |
| IP317 | IgG1-TM | YSEGT FTSDV SSYLE | AAAGG SGSTA | THTCPPC | 401 |
| | | KQAID EFIAW | SSGSG SATGG | | |
| | | LLKGG | GGAA | | |
| IP329 | IgG1-TM | YSEGT FTSDY SKLLE | G GGGGS GGGGS | no hinge | 402 |
| | | RQAID EFIAE LLKGG | GGGGS GGGGS | | |
| | | | GGGGS GGGGS A | | |
| IP330 | IgG1-TM | YSEGT FTSDY SKLLE | G GGGGS GGGGS | no hinge | 403 |
| | | RQAID EFIAL LLKGG | GGGGS GGGGS | | |
| | | | GGGGS GGGGS A | | |
| IP331 | IgG1-TM | YSEGT FTSDY SKLLE | G GGGGS GGGGS | no hinge | 404 |
| | | RQAID EFIAD LLKGG | GGGGS GGGGS | | |
| | | | GGGGS GGGGS A | | |
| IP343 | IgG1-TM | YSEGT FTSDY | G GGGGS GGGGS | no hinge | 405 |
| | | AAAAE RQAID | GGGGS GGGGS A | | |
| | | AFAAW LAAAA | | | |
| IP346 | IgG1-TM | YSEGT FTSDY SKLLE | G GGGGS GGGGS | no hinge | 406 |
| | | RQAID EFIAF LLKGG | GGGGS GGGGS | | |
| | | | GGGGS GGGGS A | | |
| IP347 | IgG1-TM | YSEGT FTSDY SKLLE | G GGGGS GGGGS | no hinge | 407 |
| | | RQAID EFIAI LLKGG | GGGGS GGGGS | | |
| | | | GGGGS GGGGS A | | |
| IP348 | IgG1-TM | YSEGT FTSDY SKLLE | G GGGGS GGGGS | no hinge | 408 |
| | | RQAID EFIAS LLKGG | GGGGS GGGGS | | |
| | | | GGGGS GGGGS A | | |
| IP349 | IgG1-TM | YSEGT FTSDY SKLLE | G GGGGS GGGGS | no hinge | 409 |
| | | RQAID EFIAN LLKGG | GGGGS GGGGS | | |
| | | | GGGGS GGGGS A | | |
| IP350 | IgG1-TM | YSEGT FTSDY SKLLE | G GGGGS GGGGS | no hinge | 410 |
| | | RQAID EFIAG LLKGG | GGGGS GGGGS | | |
| | | | GGGGS GGGGS A | | |
| IP352 | IgG1-TM | YSEGT FTSDY SKLLE | G GGGGS GGGGS | no hinge | 411 |
| | | RQAID EFIAM | GGGGS GGGGS | | |
| | | LLKGE | GGGGS GGGGS A | | |
| IP364 | IgG1-TM | YSEGT FTSDF SKLLE | AAAGG SGSTA | THTCPPC | 412 |
| | | RQAID EFIAW | SSGSG SATGG | | |
| | | LLKGG | GGAA | | |
| IP365 | IgG1-TM | YSEGM HTSDF | AAAGG SGSTA | THTCPPC | 413 |
| | | SKLLE RQAID EFIAW | SSGSG SATGG | | |
| | | LLKGG | GGAA | | |
| IP366 | IgG1-TM | YSEGT FTSDY | AAAGG SGSTA | THTCPPC | 414 |
| | | RMLKE RQAID | SSGSG SATGG | | |
| | | EFIAW LLKGG | GGAA | | |
| IP367 | IgG1-TM | YSEGT FTSDY RILKE | AAAGG SGSTA | THTCPPC | 415 |
| | | RQAID EFIAW | SSGSG SATGG | | |
| | | LLKGG | GGAA | | |
| IP368 | IgG1- | YSEGT FTSDY | G GGGGS GGGGS | CPPC | 416 |
| | TM(ΔK) | RQAID RQAID EFIAW | GGGGS GGGGS A | | |
| | | LLKGG | | | |
| IP369 | IgG1- | YSEGT FTSDY SKLLE | G GGGGS GGGGS | no hinge | 417 |
| | fusion | RQAID EFIAH LLKGG | GGGGS GGGGS | | |
| | | | GGGGS GGGGS A | | |
| IP373 | IgG1- | YSEGT HTSDF SKLLE | AAAGG SGSTA | THTCPPC | 418 |
| | fusion | RQAID EFIAW | SSGSG SATGG | | |
| | | LLKGG | GGAA | | |
| IP375 | IgG1- | YSEGT HTSLF SKLLE | AAAGG SGSTA | THTCPPC | 419 |
| | fusion | RQAID EFIAW | SSGSG SATGG | | |
| | | LLKGG | GGAA | | |
| IP376 | IgG1- | YSEGT FTSLF SKLLE | AAAGG SGSTA | THTCPPC | 420 |
| | fusion | RQAID EFIAW | SSGSG SATGG | | |
| | | LLKGG | GGAA | | |
| IP378 | IgG1-TM | YSEGT FTSLY RILLE | AAAGG SGSTA | THTCPPC | 421 |
| | | RQAID EFIAW | SSGSG SATGG | | |
| | | LLKGG | GGAA | | |
| IP385 | IgG1- | YSEGT FTSDY SIYLD | G GGGGS GGGGS | CPPC | 422 |
| | TM(ΔK) | RQAID EFIAW | GGGGS GGGGS A | | |
| | | LLKGG | | | |
| IP394 | IgG1- | YSEGM HTSDL | AAAGG SGSTA | THTCPPC | 423 |
| | fusion | SKLLE RQAID EFIAW | SSGSG SATGG | | |
| | | LLKGG | GGAA | | |
| IP395 | IgG1- | YSEGT FTSDY SKLLE | G GGGGS GGGGS | CPPC | 424 |
| | TM(ΔK) | RQAID EFIAH LLKGG | GGGGS GGGGS A | | |
| IP396 | IgG1- | YSEGT FTSDY SKLLE | G GGGGS GGGGS | CPPC | 425 |
| | TM(ΔK) | RQAID EFIAW | GGGGS GGGGS A | | |
| | | LLKGG | | | |
| IP401 | IgG1-TM | YSEGT HTSDL | AAAGG SGSTA | THTCPPC | 426 |
| | | SKLLE RQAID EFIAW | SSGSG SATGG | | |
| | | LLKGG | GGAA | | |
| IP404 | IgG1- | YSEGT FTSLY RIHKE | GS GGGGS GGGGS | CPPC | 427 |
| | TM(ΔK) | RQAID EFIAK LLKGG | GGGGS GGGGS | | |
| | | | GGGGS GGGGS | | |
| | | | GGGGS A | | |
| IP408 | IgG1- | YSEGT HTSDL RILLE | GS GGGGS GGGGS | CPPC | 428 |
| | TM(ΔK) | RQAID EFIAK LLKGG | GGGGS GGGGS | | |
| | | | GGGGS GGGGS | | |
| | | | GGGGS A | | |
| IP422 | IgG1- | YSEGT HTSDL RILLE | GS GGGGS GGGGS | CPPC | 429 |
| | TM(ΔK) | RQAID EFIAF LLKGG | GGGGS GGGGS | | |
| | | | GGGGS GGGGS | | |
| | | | GGGGS A | | |

In certain specific aspects, a GIP/GLP-1 agonist polypeptide comprising a linker, a hinge, and an Fc domain can comprise one or more of the AIS-, SIR/KIR-, or LVR-based polypeptides provided in Table 9.

TABLE 9
AIS-, SIR/KIR-, or LVR-based GIP/GLP-1 Agonist Polypeptides

| | | | | | SEQ ID |
|---|---|---|---|---|---|
| ID | IgG Fc | Agonist Peptide | Linker | Hinge | NO |
| IP171 | IgG1-TM | YSEGT FTSDV | AAAGG SGSTA | THTCPPC | 142 |
| | | SSYME RQAIS EFIAW | SSGSG SATGG | PAPEF | |
| | | LLAG | GGAA | | |
| IP176 | IgG1-TM | YSEGT FTSDV | AAAGG SGSTA | THTCPPC | 143 |
| | | SSYME RQAIS EFIAW | SSGSG SATGG | PAPEF | |
| | | LLKGP | GGAA | | |
| IP206 | IgG1-TM | HGEGT FTSDV | APPGG SGSTA | THTCPPC | 144 |
| | | SSYME AQSIR EFIAW | SSGSG SATGG | PAPEF | |
| | | LVKGP | GGAA | | |
| IP214 | IgG1-TM | HGEGT FTSDV | APPGG SGSTA | THTCPPC | 145 |
| | | SSYME AQKID | SSGSG SATGG | PAPEF | |
| | | EFIAW LVKG | GGAA | | |
| IP215 | IgG1-TM | HGEGT FTSDV | AAAGG SGSTA | THTCPPC | 146 |
| | | SSYME SQLVD | SSGSG SATGG | PAPEF | |
| | | EFIAW LVKG | GGAA | | |
| IP175 | IgG1-TM | HGEGT FTSDV | AAAGG SGSTA | THTCPPC | 147 |
| | | SSYME SQLVR | SSGSG SATGG | PAPEF | |
| | | EFIAW LVKG | GGAA | | |
| IP305 | IgG1- | YSEGT FISDY SIALA | G GGGGS GGGGS | THTCPPC | 430 |
| | TM(ΔK) | AAAVR DFVNW | GGGGS GGGGS A | | |
| | | LLAGG | | | |
| IP314 | IgG1- | YSEGT FISDY SKLLA | G GGGGS GGGGS | THTCPPC | 431 |
| | TM(ΔK) | AAAVR DFVNW | GGGGS GGGGS A | | |
| | | LLAGG | | | |
| IP318 | IgG1- | YSEGT FISDY SIAMD | G GGGGS GGGGS | THTCPPC | 432 |
| | TM(ΔK) | EEAVR DFVEW | GGGGS GGGGS A | | |
| | | LKAGG | | | |
| IP391 | IgG1-TM | HGEGT FTSDL | GS GGGGS GGGGS | CPPC | 433 |
| | | SKQME EEAVR | GGGGS GGGGS | | |
| | | LFIEW LKNGG | GGGGS A | | |
| | | PSSGA PPPS | | | |
| IP421 | IgG1- | HGEGT FTSDL | GS GGGGS GGGGS | CPPC | 434 |
| | TM(ΔK) | SKQME EECVR | GGGGS GGGGS | | |
| | | LFIEW LKNGG | GGGGS GGGGS | | |
| | | PSSGA PPPGC | GGGGS A | | |
| IP423 | IgG1- | YSEGT HTSDL RILLE | GS GGGGS GGGGS | CPPC | 435 |
| | TM(ΔK) | EEAVR LFIEF LLAGG | GGGGS GGGGS | | |
| | | | GGGGS GGGGS | | |
| | | | GGGGS A | | |

In certain specific aspects, a GIP/GLP-1 agonist polypeptide comprising a linker, a hinge, and an Fc domain can comprise one or more of the polypeptides provided in Table 10.

TABLE 10
GIP/GLP-1 Agonist Polypeptides

| | | | | | SEQ ID |
|---|---|---|---|---|---|
| ID | IgG Fc | Agonist Peptide | Linker | Hinge | NO |
| IP208 | IgG1-TM | YSEGT FTSDV SSYLE | AAAGG SGSTA | THTCPPC | 148 |
| | | GQAAK EFIAW LLAG | SSGSG SATGG | PAPEF | |
| | | | GGAA | | |
| IP204 | IgG1-TM | HGEGT FISDY SIAMD | AAAGG SGSTA | THTCPPC | 149 |
| | | KIHQQ DFVNW | SSGSG SATGG | PAPEF | |
| | | LLAQK | GGAA | | |
| IP199 | IgG1-TM | HGEGT FTSDV | AAAGG SGSTA | THTCPPC | 150 |
| | | SSYLE GQAAK | SSGSG SATGG | PAPEF | |
| | | EFIAW LLAG | GGAA | | |
| IP173 | IgG1-TM | YSEGT FTSDV SSYLE | AAAGG SGSTA | THTCPPC | 151 |
| | | EQAAK EFIAW LLAG | SSGSG SATGG | PAPEF | |
| | | | GGAA | | |
| IP150 | IgG1-TM | YSEGT FTSDV SSYLE | APPGG SGSTA | THTCPPC | 152 |
| | | GQAAK EFIAW | SSGSG SATGG | PAPEF | |
| | | LVAGG | GGAA | | |
| IP174 | IgG1-TM | HGEGT FTSDV | AAAGG SGSTA | THTCPPC | 153 |
| | | SSYLE EEAAK | SSGSG SATGG | PAPEF | |
| | | EFIAW LVAGG | GGAA | | |
| IP205 | IgG1-TM | YSEGT FTSDL | AAAGG SGSTA | THTCPPC | 154 |
| | | SKQME EEAVR | SSGSG SATGG | PAPEF | |
| | | LFIEW LKNGG | GGAA | | |
| | | PSSGA PPPS | | | |
| IP127 | IgG1-TM | YSEGT FTSDV SSYLE | AAAGG SGSTA | THTCPPC | 155 |
| | | EEAVR EFIEW | SSGSG SATGG | PAPEF | |
| | | LLAGG | GGAA | | |
| IP200 | IgG1-TM | YSQGT FTSDY SKILE | AAAGG SGSTA | THTCPPC | 156 |
| | | EEAVR LFIEW | SSGSG SATGG | PAPEF | |
| | | LVKGP | GGAA | | |
| IP015 | IgG1-TM | HGEGT FTSDV | AAAGG SGSTA | THTCPPC | 157 |
| | | SSYLE GQAAK | SSGSG SATGG | PAPEF | |
| | | EFIAW LVKG | GGAA | | |
| IP231 | IgG1-TM | HGEGT FTSDV | G GGGGS GGGGS | THTCPPC | 158 |
| | | SSYLE EQAAK | GGGGS GGGGS A | | |
| | | EFIAW LVKG | | | |
| IP233 | IgG1-TM | HGEGT FTSDV | AAAGG SGSTA | THTCPPC | 159 |
| | | SSYLE EQAAK | SSGSG SATGG | PAPEF | |
| | | EFIAW LVKG | GGAA | | |
| IP132 | IgG1-TM | HGEGT FTSDV | G GGGGS GGGGS | THTCPPC | 160 |
| | | SSYLE GQAAK | GGGGS GGGGS A | PAPEF | |
| | | EFIAW LVKG | | | |
| IP146 | IgG1-TM | HGEGT FTSDV | AAAGG SGSTA | CPPCPAP | 161 |
| | | SSYLE GQAAK | SSGSG SATGG | EF | |
| | | EFIAW LVKG | GGAA | | |
| IP187 | IgG1-TM | HGEGT FTSDV | APP G GGGGS | THTCPPC | 162 |
| | | SSYLE GQAAK | GGGGS GGGGS | PAPEF | |
| | | EFIAW LVKG | GGGGS A | | |
| IP191 | IgG1-TM | HGEGT FTSDV | AAAGG SGSTA | TYTCPPC | 163 |
| | | SSYLE GQAAK | SSGSG SATGG | PAPEF | |
| | | EFIAW LVKG | GGAA | | |
| IP192 | IgG1-TM | HGEGT FTSDV | AAAGG SGSTA | TSTCPPC | 164 |
| | | SSYLE GQAAK | SSGSG SATGG | PAPEF | |
| | | EFIAW LVKG | GGAA | | |
| IP193 | IgG1-TM | HGEGT FTSDV | AAAGG SGSTA | CPPCPAP | 165 |
| | | SSYLE GQAAK | SSGSG SATGG GG | EF | |
| | | EFIAW LVKG | | | |
| IP194 | IgG1-TM | HGEGT FTSDV | AAAGG SGSTA | PPCPPCP | 166 |
| | | SSYLE GQAAK | SSGSG SATGG GG | APEF | |
| | | EFIAW LVKG | | | |
| IP195 | IgG1-TM | HGEGT FTSDV | AAAGG SGSTA | ESKYGPP | 167 |
| | | SSYLE GQAAK | SSGSG SATGG GG | CPPCPAP | |
| | | EFIAW LVKG | | EF | |
| IP196 | IgG1-TM | HGEGT FTSDV | AAAGG SGSTA | APEF | 168 |
| | | SSYLE GQAAK | SSGSG SATGG GG | | |
| | | EFIAW LVKG | | | |
| IP203 | IgG1-TM | HGEGT FTSDV | AAAGG SGSTA | THTCPPC | 169 |
| | | SSYLE GQAAK | SSGSG SATGG | PAPEF | |
| | | EFIAW LVKGG | GGAA | | |
| IP230 | IgG1FQQ | HGEGT FTSDV | AAAGG SGSTA | THTCPPC | 170 |
| | | SSYLE GQAAK | SSGSG SATGG | PAPEF | |
| | | EFIAW LVKG | GGAA | | |
| IP238 | IgG4 | HGEGT FTSDV | G GGGGS GGGGS | ESKYGPP | 171 |
| | | SSYLE GQAAK | GGGGS A | CPPC | |
| | | EFIAW LVKG | | | |
| IP122 | IgG1-TM | HGEGT FTSDL | AAAGG SGSTA | THTCPPC | 172 |
| | | SKQME EEAVR | SSGSG SATGG | PAPEF | |
| | | LFIEW LKNGG | GGAA | | |
| | | PSSGA PPPS | | | |
| IP018 | IgG1-TM | YSEGT FISDY SIAMD | AAAGG SGSTA | THTCPPC | 173 |
| | | KIHQQ DFVNW | SSGSG SATGG | PAPEF | |
| | | LLAQK | GGAA | | |
| IP188 | IgG1-TM | YSEGT FISDY SIAMD | APP G GGGGS | THTCPPC | 174 |
| | | KIHQQ DFVNW | GGGGS GGGGS | PAPEF | |
| | | LLAQK | GGGGS A | | |
| IP207 | IgG1 | YAEGT FISDY SIAMD | AAAGG SGSTA | THTCPPC | 175 |
| | | KIHQQ DFVNW | SSGSG SATGG | PAPELL | |
| | | LLAQK GKKND | GGAA | | |
| | | WKHNI TQ | | | |
| IP302 | IgG1- | YSEGT FISDY SIALA | G GGGGS GGGGS | THTCPPC | 436 |
| | TM(ΔK) | AAAQQ DFVNW | GGGGS GGGGS A | | |
| | | LLAGG | | | |
| IP308 | IgG1- | YSEGT FISDY SIALA | G GGGGS GGGGS | THTCPPC | 437 |
| | TM(ΔK) | AAAAK DFVNW | GGGGS GGGGS A | | |
| | | LLAGG | | | |
| IP311 | IgG1- | YSEGT FISDY SIALA | G GGGGS GGGGS | THTCPPC | 438 |
| | TM(ΔK) | AAAID DFVNW | GGGGS GGGGS A | | |
| | | LLAGG | | | |
| IP319 | IgG1- | YSEGT FTSDY SKLLE | G GGGGS GGGGS | CPPC | 439 |
| | TM(ΔK) | AAAAA AFAAW | GGGGS GGGGS A | | |
| | | LAAAA | | | |
| IP320 | IgG1- | YSEGT FTSDY | G GGGGS GGGGS | CPPC | 440 |
| | TM(ΔK) | AAAAE AAAAA | GGGGS GGGGS A | | |
| | | AFAAW LAAAA | | | |
| IP332 | IgG1-TM | YSEGT FTSDY SIALA | G GGGGS GGGGS | no hinge | 441 |
| | | AAAQQ DFVNW | GGGGS GGGGS | | |
| | | LLAGG | GGGGS GGGGS A | | |
| IP333 | IgG1-TM | YSEGT FISDY SIALA | G GGGGS GGGGS | no hinge | 442 |
| | | AAAQQ DFVNW | GGGGS GGGGS | | |
| | | LLKGG | GGGGS GGGGS A | | |
| IP334 | IgG1-TM | YSEGT FTSDY SIALA | G GGGGS GGGGS | no hinge | 443 |
| | | AAAQQ DFVNW | GGGGS GGGGS | | |
| | | LLKGG | GGGGS GGGGS A | | |
| IP342 | IgG1-TM | YSEGT FTSDY | G GGGGS GGGGS | CPPC | 444 |
| | | SAAAE AAAAA | GGGGS GGGGS A | | |
| | | AFAAW LAAAA | | | |
| IP353 | IgG1- | YSEGT FTSDY | G GGGGS GGGGS | CPPC | 445 |
| | TM(ΔK) | AAAAE AAAAA | GGGGS GGGGS A | | |
| | | AFAAW LAKAA | | | |
| IP321 | IgG1-TM | YSEGT FISDY SIAMD | AAAGG SGSTA | THTCPPC | 446 |
| | | KIHQQ DFVNW | SSGSG SATGG | | |
| | | LLAGG | GGAA | | |
| IP335 | IgG1-TM | YSEGT FISDY SIAMD | G GGGGS GGGGS | no hinge | 447 |
| | | KIHQQ DFVNW | GGGGS GGGGS A | | |
| | | LLAQK | | | |
| IP336 | IgG1-TM | YSEGT FISDY SIAMD | G GGGGS GGGGS | no hinge | 448 |
| | | KIHQQ DFVNW | GGGGS GGGGS | | |
| | | LLAQK | GGGGS GGGGS A | | |
| IP337 | IgG1-TM | YSEGT FISDY SIAMD | G GGGGS GGGGS | no hinge | 449 |
| | | KIHQQ DFVNW | GGGGS GGGGS | | |
| | | LLAQK | GGGGS GGGGS | | |
| | | | GGGGS A | | |
| IP338 | IgG1-TM | YSEGT FISDY SIAMD | G GGGGS GGGGS | no hinge | 450 |
| | | KIHQQ DFVNW | GGGGS GGGGS | | |
| | | LLAQK | GGGGS GGGGS | | |
| | | | GGGGS GGGGS A | | |
| IP344 | IgG1-TM | YSEGT FISDY SIAMD | G GGGGS GGGGS | no hinge | 451 |
| | | KIHQQ DFVNW | GGGGS GGGGS | | |
| | | LLAQK | GGGGS A | | |
| IP351 | IgG1- | YSEGT FISDY SIAMD | GGGGS GGGGS | CPPC | 452 |
| | TM(ΔK) | KIHQQ DFVNW | GGGGS GGGGS A | | |
| | | LLAQK PSSGA PPPS | | | |
| IP386 | IgG1-TM | YSEGT FISDY SIAMD | G GGGGS GGGGS | CPPC | 453 |
| | | KIHQQ DFVNW | GGGGS GGGGS A | | |
| | | LLAQK | | | |
| IP387 | IgG1-TM | YSEGT FISDY SIAMD | G GGGGS GGGGS | CPPC | 454 |
| | | KIHQQ DFVNW | GGGGS GGGGS | | |
| | | LLAQK | GGGGS GGGGS | | |
| | | | GGGGS GGGGS A | | |
| IP388 | IgG1-TM | YSEGT FISDY SIAMD | G GGGGS GGGGS | CPPC | 455 |
| | | KIHQQ DFVNW | GGGGS GGGGS | | |
| | | LLAQK | GGGGS GGGGS A | | |
| IP389 | IgG1-TM | YSEGT FISDY SIAMD | G GGGGS GGGGS | CPPC | 456 |
| | | KIHQQ DFVNW | GGGGS GGGGS | | |
| | | LLAQK | GGGGS GGGGS | | |
| | | | GGGGS A | | |
| IP392 | IgG1-TM | YSEGT FISDY SIAMD | GGGGS GGGGS | CPPC | 457 |
| | | KIHQQ DFVNW | GGGGS GGGGS | | |
| | | LLAQK GAAND | GGGGS GGGGS A | | |
| | | WKHNI TQ | | | |
| IP398 | IgG1-TM | YSEGT FISDY SIAMD | GGGGS GGGGS | CPPC | 458 |
| | | KIHQQ DFVNW | GGGGS GGGGS | | |
| | | LLAQK GKKND | GGGGS GGGGS A | | |
| | | WKHNI TQ | | | |
| IP402 | IgG1- | YSEGT FISDY SIAMD | GS GGGGS GGGGS | CPPC | 459 |
| | TM(ΔK) | KIHQQ DFVNW | GGGGS GGGGS | | |
| | | LLAQK | GGGGS GGGGS | | |
| | | | GGGGS A | | |
| IP419 | IgG1- | YSEGT FISDY SIAMD | G GGGGS GGGGS | CPPC | 460 |
| | TM(ΔK) | KIHQQ DFVNW | GGGGS GGGGS | | |
| | | LLAQK | GGGGS GGGGS A | | |

In certain aspects, a GIP/GLP-1 agonist polypeptide as described above can form multimers. For example, two or more GIP/GLP-1 monomer polypeptides can be joined through disulfide bonds, through cysteines contained, e.g., in the linker or hinge regions of two or more polypeptide monomers. In certain aspects two or more monomer can be identical, resulting in a homomonomer, e.g., a homodimer. Alternatively the two or more polypeptide monomers can be different, resulting in a heteromonomer, e.g., a heterodimer.

In certain aspects, GIP/GLP-1 agonist polypeptides as disclosed have desirable potencies at the GIP and GLP-1 receptors for controlling symptoms of a hypoglycemic condition, e.g., type-2 diabetes. In certain aspects, GIP/GLP-1 agonist polypeptides as disclosed exhibit in vitro potencies at the GLP-1 receptor as shown by an EC50 in the BSA-cAMP assay of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, less than 2 pM, or less than 1 pM.

In certain aspects, GIP/GLP-1 agonist polypeptides as disclosed exhibit in vitro potencies at the GIP receptor as shown by an EC50 in the BSA-cAMP assay of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, less than 2 pM, or less than 1 pM.

Activity at the glucagon receptor (GCGr) (which promotes glucose production) can be undesirable in the control of hypoglycemic conditions, e.g., type-2 diabetes. See, e.g, D'Alessio, D., Diabetes Obes. Metab. Suppl 1:126-32 (2011). Accordingly, In certain aspects, a GIP/GLP-1 agonist polypeptide as disclosed herein exhibit reduced potency for GCGr relative to either GIPr or GLP-1r, or both GIPr and GLP-1r. For example, the polypeptide can exhibit an EC50 for GCGr, as measured by the BSA-cAMP assay, of at least 10-fold higher, at least 100-fold higher, at least 1000-fold higher, or at least 10,000 or more-fold higher than the polypeptide's EC50 for GIPr, GLP-1r, or both, as measured by the BSA-cAMP assay.

In certain aspects, GIP/GLP-1 agonist polypeptides as disclosed exhibit in vitro potencies at GCGr as shown by an EC50 in the BSA-cAMP assay of greater than 0.1 nM, greater than 1 nM, greater than 5 nM, greater than 10 nM, greater than 50 nM, greater than 100 nM, greater than 200 nM, greater than 300 nM, greater than 400 nM, greater than 500 nM, greater than 600 nM, greater than 700 nM, greater than 800 nM, greater than 900 nM, greater than 1000 nM, greater than 2000 nM, greater than 3000 nM, greater than 4000 nM, greater than 5000 nM, greater than 6000 nM, greater than 7000 nM, greater than 8000 nM, greater than 9000 nM, or greater than 10,000 nM.

Methods of Making.

This disclosure provides a method of making a GIP/GLP-1 agonist polypeptide. GIP/GLP-1 agonist polypeptides provided herein can be made by any suitable method. For example, GIP/GLP-1 agonist polypeptides provided herein can be produced recombinantly using a convenient vector/host cell combination as would be well known to the person of ordinary skill in the art. A variety of methods are available for recombinantly producing GIP/GLP-1 agonist polypeptides. Generally, a polynucleotide sequence encoding the GIP/GLP-1 agonist polypeptide is inserted into an appropriate expression vehicle, e.g., a vector that contains the necessary elements for the transcription and translation of the inserted coding sequence. The nucleic acid encoding the GIP/GLP-1 agonist polypeptide is inserted into the vector in proper reading frame. The expression vector is then transfected into a suitable host cell that will express the GIP/GLP-1 agonist polypeptide. Suitable host cells include without limitation bacteria, yeast, or mammalian cells. A variety of commercially available host-expression vector systems can be utilized to express the GIP/GLP-1 agonist polypeptides described herein.

The recombinant expression of a GIP/GLP-1 agonist polypeptide, derivative, analog or fragment thereof as described herein can be accomplished through the construction of an expression vector containing a polynucleotide that encodes the polypeptide. Once a polynucleotide encoding the GIP/GLP-1 agonist polypeptide has been obtained, the vector for the production of the polypeptide can be produced by recombinant DNA technology using techniques well known in the art.

Thus, methods for preparing a protein by expressing a polynucleotide containing a nucleotide sequence encoding a GIP/GLP-1 agonist polypeptide are described herein. Methods that are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Thus, replicable vectors are provided that comprise a nucleotide sequence encoding a GIP/GLP-1 agonist polypeptide, operably linked to a promoter.

The expression vector is transferred to a host cell by conventional techniques and the transfected cells are then cultured by conventional techniques to produce a GIP/GLP-1 agonist polypeptide. Thus, host cells are provided that contain a polynucleotide encoding a GIP/GLP-1 agonist polypeptide, operably linked to a heterologous promoter.

A variety of host-expression vector systems can be utilized to express a GIP/GLP-1 agonist polypeptide. Such host-expression systems represent vehicles by which the coding sequences of interest can be produced and subsequently purified, but also represent cells which can, when transformed or transfected with the appropriate nucleotide coding sequences, express a polypeptide comprising a GIP/GLP-1 agonist polypeptide in situ. These include but are not limited to microorganisms such as bacteria (e.g., E. coli and B. subtilis) transformed with recombinant bacteriophage DNA, plasmid DNA or cosmid DNA expression vectors containing a sequence or sequences encoding a GIP/GLP-1 agonist polypeptide; yeast (e.g., Saccharomyces Pichia) transformed with recombinant yeast expression vectors containing a sequence or sequences encoding a GIP/GLP-1 agonist polypeptide; insect cell systems infected with recombinant virus expression vectors (e.g., baculovirus) containing a sequence or sequences encoding a GIP/GLP-1 agonist polypeptide; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a sequence or sequences encoding a GIP/GLP-1 agonist polypeptide; or mammalian cell systems (e.g., COS, CHO, BHK, 293, NS0, 3T3 cells) harboring recombinant expression constructs containing promoters derived from the genome of mammalian cells or from mammalian viruses.

A host cell strain can be chosen that modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Eukaryotic host cells that possess the cellular machinery for proper processing of the primary transcript, glycosylation, and phosphorylation of the gene product can be used. Such mammalian host cells include but are not limited to CHO, VERY, BHK, HeLa, COS, MDCK, 293, 3T3, W138, BT483, Hs578T, HTB2, BT20 and T47D, NS0, CRL7O3O and HsS78Bst cells.

For long-term, high-yield production of recombinant proteins, stable expression is often preferred. For example, cell lines that stably express a GIP/GLP-1 agonist polypeptide can be engineered using methods known in the art.

Once a GIP/GLP-1 agonist polypeptide has been produced by recombinant expression, it can be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

Alternatively, a GIP/GLP-1 agonist polypeptides provided herein can be chemically synthesized by methods well known to those of ordinary skill in the art, e.g., by solid phase synthesis as described by Merrifield (1963, J. Am. Chem. Soc. 85:2149-2154). Solid phase peptide synthesis can be accomplished, e.g., by using automated synthesizers, using standard reagents.

A GIP/GLP-1 agonist polypeptide can be characterized in a variety of ways. In particular, a GIP/GLP-1 agonist polypeptide can be assayed for potency in a cAMP assay as described elsewhere herein.

Modifications, Conjugates, Fusions, and Derivations.

In certain aspects, GIP/GLP-1 agonist polypeptides provided herein are stabilized via amino acid modifications. In certain aspects, the carboxyl group of the C-terminal amino acid is amidated. In certain aspects, the C-terminal amino acid is amidated glycine. In certain aspects the C-terminal glycine is the unmodified acid. In certain aspects, GIP/GLP-1 agonist polypeptides are provided in which one or more amino acid residues are acylated. For example, in certain aspects GIP/GLP-1 agonist polypeptides provided herein contain one or more lysine residues, in which a palmitoyl moiety is attached to the N(epsilon) group. In certain aspects a linker is incorporated between lysine and the palmitoyl group. This linker can be a gamma glutamic acid group, or an alternative linker such as, but not limited to, beta alanine and aminohexanoic acid. Different acylation methods may be used such as addition of cholesterol or myristoyl groups.

A GIP/GLP-1 agonist polypeptide as disclosed herein can be associated with a heterologous moiety, e.g., to extend half-life. The heterologous moiety can be a protein, a peptide, a protein domain, a linker, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of such moieties.

For example, a GIP/GLP-1 agonist polypeptide can be fused with a heterologous polypeptide, e.g., a linker, a hinge, an Fc, or a combination thereof, as described above or in additional ways. The peptides can be fused to proteins, either through recombinant gene fusion and expression or by chemical conjugation. Proteins that are suitable as partners for fusion include, without limitation, serum albumin, e.g., human serum albumin, antibodies and antibody fragments including fusion to the Fc portion of the antibodies (as described above). GLP-1 has been fused to these proteins with retention of potency (L. Baggio et al., Diabetes 53:2492-2500 (2004); P. Barrington et al., Diabetes, Obesity and Metabolism 13:426-433 (2011); P. Paulik et al., American Diabetes Association 2012, Poster 1946). Extended recombinant peptide sequences have also been described to give the peptide high molecular mass (V. Schellenberger et al., Nature Biotechnol 27:1186-1190 (2009). In certain aspects GIP/GLP-1 agonist polypeptides are incorporated as the N-terminal part of a fusion protein, with the fusion partner, e.g., an Fc domain as described above, or an albumin domain, at the C-terminal end. GIP/GLP-1 agonist polypeptides as described herein can also be fused to peptides or protein domains, such as ‘Albudabs’ that have affinity for human serum albumin (M. S. Dennis et al., J Biol Chem 277:35035-35043 (2002); A. Walker et al., Protein Eng Design Selection 23:271-278 (2010)). Methods for fusing GIP/GLP-1 agonist polypeptides as disclosed herein with a heterologous polypeptide, e.g., albumin or an Fc region, are well known to those of ordinary skill in the art.

Other heterologous moieties can be conjugated to GIP/GLP-1 agonist polypeptides to further stabilize or increase half-life. For chemical fusion, certain aspects feature maintenance of a free N-terminus, but alternative points for derivatization can be made. A further alternative method is to derivatize the peptide with a large chemical moiety such as high molecular weight polyethylene glycol (PEG). A “pegylated GIP/GLP-1 agonist polypeptide” has a PEG chain covalently bound thereto. Derivatization of GIP/GLP-1 agonist polypeptides, e.g., pegylation, can be done at the lysine that is palmitoylated, or alternatively at a residue such as cysteine, that is substituted or incorporated by extension to allow derivatization. GIP/GLP-1 agonist polypeptide formats above can be characterized in vitro and/or in vivo for relative potency and the balance between GLP-1 and GIP receptor activation.

The general term “polyethylene glycol chain” or “PEG chain”, refers to mixtures of condensation polymers of ethylene oxide and water, in a branched or straight chain, represented by the general formula H(OCH₂CH₂)ₙOH, where n is an integer of 3, 4, 5, 6, 7, 8, 9, or more. PEG chains include polymers of ethylene glycol with an average total molecular weight selected from the range of about 500 to about 40,000 Daltons. The average molecular weight of a PEG chain is indicated by a number. For example, PEG-5,000 refers to polyethylene glycol chain having a total molecular weight average of about 5,000.

PEGylation can be carried out by any of the PEGylation reactions known in the art. See, e.g., Focus on Growth Factors, 3: 4-10, 1992 and European patent applications EP 0 154 316 and EP 0 401 384. PEGylation may be carried out using an acylation reaction or an alkylation reaction with a reactive polyethylene glycol molecule (or an analogous reactive water-soluble polymer).

Methods for preparing a PEGylated GIP/GLP-1 agonist polypeptides generally include the steps of (a) reacting a GIP/GLP-1 agonist polypeptide or with polyethylene glycol (such as a reactive ester or aldehyde derivative of PEG) under conditions whereby the molecule becomes attached to one or more PEG groups, and (b) obtaining the reaction product(s).

### Formulations and Pharmaceutical Compositions

In certain aspects, GIP/GLP-1 agonist polypeptides provided herein possess one or more criteria of acceptable solubility, ease in formulatability, plasma stability, and improved pharmacokinetic properties. In certain aspects, GIP/GLP-1 agonist polypeptides as disclosed are soluble in standard buffers over a broad pH range.

In certain aspects, GIP/GLP-1 agonist polypeptides as disclosed are acceptably stable against proteases in serum or plasma. Common degradation products of GIP or GLP-1 include +1 products (acid) and the DPP IV-cleavage products. Cleavage products arise from the action of proteases, e.g., DPP IV in plasma. In certain aspects, GIP/GLP-1 agonist polypeptides as disclosed are remain stable in plasma at levels up to 30%, 40%, 50%, 60%, 70%, 80%, 90%, or 100% after 24, 36 hours, 48 hours, or more hours in plasma at 37° C.

Further provided are compositions, e.g., pharmaceutical compositions, that contain an effective amount of a GIP/GLP-1 agonist polypeptide as provided herein, formulated for the treatment of metabolic diseases, e.g., obesity.

Compositions of the disclosure can be formulated according to known methods. Suitable preparation methods are described, for example, in Remington's Pharmaceutical Sciences, 19th Edition, A. R. Gennaro, ed., Mack Publishing Co., Easton, Pa. (1995), which is incorporated herein by reference in its entirety. Composition can be in a variety of forms, including, but not limited to an aqueous solution, an emulsion, a gel, a suspension, lyophilized form, or any other form known in the art. In addition, the composition can contain pharmaceutically acceptable additives including, for example, diluents, binders, stabilizers, and preservatives. Once formulated, compositions of the invention can be administered directly to the subject.

Carriers that can be used with compositions of the invention are well known in the art, and include, without limitation, e.g., thyroglobulin, albumins such as human serum albumin, tetanus toxoid, and polyamino acids such as poly L-lysine, poly L-glutamic acid, influenza, hepatitis B virus core protein, and the like. A variety of aqueous carriers can be used, e.g., water, buffered water, 0.8% saline, 0.3% glycine, hyaluronic acid and the like. Compositions can be sterilized by conventional, well known sterilization techniques, or can be sterile filtered. A resulting composition can be packaged for use as is, or lyophilized, the lyophilized preparation being combined with a sterile solution prior to administration. Compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamineoleate, etc.

### Method of Treating Hypoglycemic Conditions, e.g., Type-2 Diabetes, Model Systems.

GIP/GLP-1 agonist polypeptides can combine the effect of GIP and GLP-1 to provide one or more of prevention or modulation of hyperglycemia, promotion of insulin synthesis, inhibition of glucagon synthesis, an increase in β-cell mass, weight loss or weight maintenance (e.g., prevention of weight gain), reduction in food intake, modulation of gastric acid secretion, or modulation of gastric emptying.

This disclosure provides a method of treating a hypoglycemic condition, e.g., type-2 diabetes, comprising administering to a subject in need of treatment a GIP/GLP-1 agonist polypeptide as disclosed herein. Further provided is a GIP/GLP-1 agonist polypeptide for treatment of a hypoglycemic condition, e.g., type-2 diabetes. Further provided is use of a GIP/GLP-1 agonist polypeptide as provided herein in the manufacture of a medicament for the treatment of a hypoglycemic condition, e.g., type-2 diabetes.

GIP/GLP-1 agonist polypeptides provided herein can be administered for glycemic control, promoting insulin production, promoting β-cell mass, promoting weight loss, or reducing excess body weight. In addition, GIP/GLP-1 agonist polypeptides provided herein can be used for treatment of related disorders. Examples of related disorders include without limitation: insulin resistance, glucose intolerance, pre-diabetes, increased fasting glucose, hypertension, dyslipidemia (or a combination of these metabolic risk factors), glucagonomas, cardiovascular diseases such as congestive heart failure, atherosclerois, arteriosclerosis, coronary heart disease, or peripheral artery disease, stroke, respiratory dysfunction, or renal disease.

“Treatment” is an approach for obtaining beneficial or desired clinical results. As provided herein, beneficial or desired clinical results from the disclosed GIP/GLP-1 agonist polypeptides include, without limitation, stabilized serum glucose and serum insulin levels, increased β-cell mass, or amelioration, palliation, stabilization, diminishment of weight gain. “Treatment” refers to both therapeutic treatment and prophylactic or preventative measures in certain aspects. Those in need of treatment include those already with the disorder as well as those in which the disorder is to be prevented. By treatment is meant improved glycemic control in type-2 diabetes, and is not necessarily meant to imply complete cure of the relevant condition.

The route of administration of GIP/GLP-1 agonist polypeptides provided herein can be, for example, oral, parenteral, by inhalation or topical. The term parenteral as used herein includes, e.g., intravenous, intraarterial, intraperitoneal, intramuscular, subcutaneous, rectal, or vaginal administration. Another example of a form for administration is a solution for injection, in particular for intravenous or intraarterial injection or drip. GIP/GLP-1 agonist polypeptides provided herein can be administered as a single dose or as multiple doses. In certain aspects, a GIP/GLP-1 agonist polypeptide is administered orally or by subcutaneous injection.

Parenteral formulations can be a single bolus dose, an infusion or a loading bolus dose followed with a maintenance dose. These compositions can be administered at specific fixed or variable intervals, e.g., once a day, or on an “as needed” basis, e.g., based on patient-initiated blood glucose measurements. Dosage regimens also can be adjusted to provide the optimum desired response (e.g., a therapeutic or prophylactic response).

The amount of a GIP/GLP-1 agonist polypeptide to be administered can be readily determined by one of ordinary skill in the art without undue experimentation given the disclosure herein. Factors influencing the mode of administration and the respective amount of a GIP/GLP-1 agonist polypeptide include, but are not limited to, the severity of the disease (e.g., the extent of obesity), the subject's history, and the age, height, weight, health, and physical condition of the subject undergoing therapy. Similarly, the amount of a GIP/GLP-1 agonist polypeptide to be administered will be dependent upon the mode of administration and whether the subject will undergo a single dose or multiple doses of this agent. In certain aspects, GIP/GLP-1 agonist polypeptides provided herein can be administered once per day via injection.

In certain aspects a GIP/GLP1 agonist polypeptide as provided herein can be administered in combination with one or more additional therapies. The additional therapy can include one or more existing standard therapies type-2 diabetes or other hypoglycemic condition, or new therapies. In certain aspects, the one or more additional therapies can include, without limitation, blood sugar monitoring, diet modifications, exercise, insulin, a thiazolidinedione, a sulfonylurea, an incretin, metformin, a glyburide, a dipeptidyl peptidase 4 inhibitor, a bile acid sequestrant, or any combination thereof.

### Kits

In yet other aspects, the present disclosure provides kits comprising GIP/GLP-1 agonist polypeptides, which can be used to perform the methods described herein. In certain aspects, a kit comprises a GIP/GLP-1 agonist polypeptide disclosed herein in one or more containers. A kit as provided herein can contain additional compositions for combination therapies. One skilled in the art will readily recognize that the disclosed GIP/GLP-1 agonist polypeptides can be readily incorporated into one of the established kit formats that are well known in the art.

### EXAMPLES

### Example 1

### Synthesis, Modifications, and Characterization of GIP/GLP-1 Agonist Polypeptides

Dual-active GIP/GLP-1 agonist polypeptides were prepared according to the following methods. GIP/GLP dual agonist peptides were designed by starting with various different backbone peptides, including GLP-1, GIP, and Exendin-4. Rational substitutions were made to allow specificity at GLP-1r and GIPr. Alternatively, the backbone peptides were subjected to random mutagenesis, expressed in phage, and selected based on binding to the various receptors.

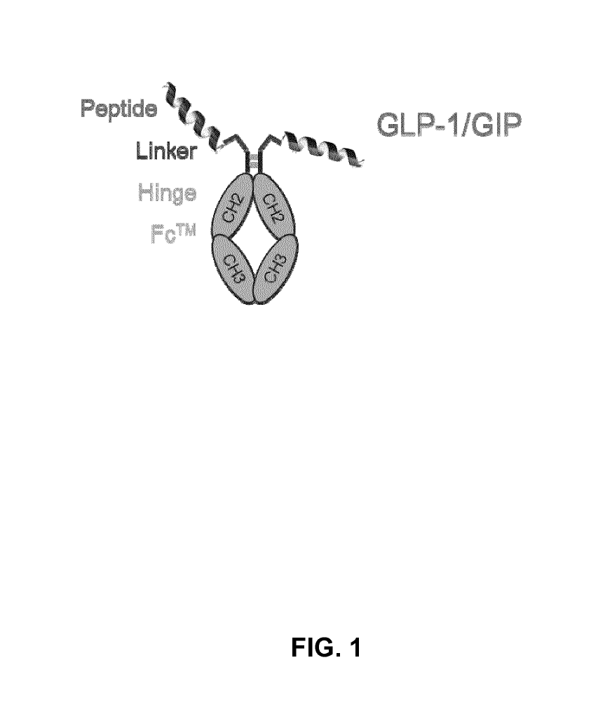

A large number of peptides were produced, and were expressed as a fusion protein, extending from N-terminus to C-terminus: peptide-linker-hinge-Fc (CH2 and CH3 regions, from either IgG1 or IgG4, with various mutations to improve stability and reduce effector functions, such as IgG1-TM, or IgG1-FQQ, as described elsewhere herein). Such a construct will form a dimer through disulfide linkages in the hinge region. Various peptide-linker-hinge-Fc combinations were constructed as shown in Tables 7-10. An exemplary bivalent GIP/GLP-1 agonist polypeptide composition is shown in FIG. 1.

The various GIP/GLP-1 agonist polypeptides were constructed and expressed in CHO cells by standard methods using the following procedures. CEP6 cells (MedImmune) were maintained in CD-CHO medium (Invitrogen) containing 100 mg/l Hygromycin B and 25 mg/l MSX. Cells were received one day prior to transfection at approximately 1.0×10⁶ viable cells/ml, having been passaged twice without Hygromycin B selection prior to delivery. The cells generally double in number after 24 hours reaching the required 2.0×10⁶ viable cells/ml for use on the day of transfection.

Aliquots of 30 ml of CEP6 cells at 2.0×10⁶ viable cells/ml were transferred into 125 ml flasks. Place The flasks were placed in a 37° C. shaking incubator set to 140 rpm with 5% CO₂ and 80% humidity.

DNA was prepared by adding a total of 20 μg (total) of plasmid DNA to a 1.5 ml Eppendorf tube. The volume in each tube was made up to a total of 500 μl with 150 mM sodium chloride solution. The PEI was prepared by adding a 200 μl volume of PEI Max working solution (1 mg/ml) to a 1.5 ml Eppendorf tube. The volume in each tube was made up to a total of 500 μl with 150 mM sodium chloride solution. The PEI Max was added to the DNA and vortexed for 10 sec (DNA:PEI Max ratio 1:10). The PEI Max/DNA complex was incubated at room temperature for 1 min, and then added to the cells.

Each flask was incubated in a 37° C. shaking incubator set to 140 rpm with 5% CO₂ and 80% humidity. After a minimum of 4 hours, a 9 ml volume of feed (30% initial volume) was added.

The cultures were incubated for seven days in a 34° C. shaking incubator set to 140 rpm with 5% CO₂ and 80% humidity.

Following incubation, the CEP6 cells were pelleted by transferring the content of the flasks to 50 ml Falcon tubes and centrifuging at 1500 rpm for 20 min. The cells were harvested from the flasks by filtering the supernatant through a 0.22 μm Steriflip filter.

Proteins were purified using the AKTAxpress by affinity chromatography with MabSelectSuRe columns, where the Fc region of the protein binds to the column matrix. Columns were equilibrated in 1×DPBS, (Gibco, Invitrogen. Cat No:14190-094). During sample loading the protein binds to the column, with the unbound sample being washed. Elution of the bound material was done with 0.1M Sodium citrate, pH3 into collection blocks containing one fifth vol. 1M Tris-HCl, pH 9.0.

Finally, purified protein was buffer exchanged on PD 10 columns equilibrated with PBS.

### Example 2

### GIP and GLP-1 Receptor Mediated cAMP Production

Biological Activity of Peptides in Cell-Based cAMP Activity Assay.

The biological activity of GIP/GLP-1 agonist polypeptides produced by the method of Example 1 were tested for biological activity, e.g., stimulation of one or more cellular receptor responses, by the following methods. Stable cell lines expressing human, mouse, or rat GLP-1 receptor (GLP-1r), GIP receptor (GIPr) or glucagon receptor (GCGr) were generated in HEK293s or CHO cells by standard methods. For human GIPr, two different lines were used, shown as “hGIPr” and “hGIPr (C3)” in Table 11. Peptide activation of these various receptors results in downstream production of cAMP second messenger that can be measured in a functional activity assay.

One basic cAMP assays was performed, differing in the medium used for the assay, the “BSA-cAMP assay”:

BSA-based assay medium: 0.1% bovine serum albumin (BSA) in

Hanks Balanced Salt Solution (GIBCO or Sigma), containing 0.5 mM IBMX (Sigma #17018);

In certain aspects, the assay can also be carried out in medium supplemented with serum albumin that corresponds to the receptor being tested, e.g., human serum albumin for testing cAMP activity at hGIPr or hGLP-1r, rat serum albumin for testing cAMP activity at rGIPr or rGLP-1r, and so on. Low protein binding 384-well plates (Greiner #781280) were used to perform eleven 1 in 5 serial dilutions of test samples that were made in assay medium. All sample dilutions were made in duplicate.

A frozen cryo-vial of cells expressing the receptor of interest was thawed rapidly in a water-bath, transferred to pre-warmed assay media and spun at 240×g for 5 minutes. Cells were re-suspended in assay buffer at an optimized concentration (e.g., hGLP-1r and hGIPr cells at 0.5×10⁵ cells/ml).

From the dilution plate, a 5 μL replica was stamped onto a black shallow-well u-bottom 384-well plate (Corning #3676). To this, 5 μL cell suspension was added and the plates incubated at room temperature for 30 minutes.

cAMP levels were measured using a commercially available cAMP dynamic 2 HTRF kit (Cisbio, Cat #62AM4PEJ), following the two step protocol as per manufacturer's recommendations. In brief; anti-cAMP cryptate (donor fluorophore) and cAMP-d2 (acceptor fluorophore) were made up separately by diluting each 1/20 in conjugate & lysis buffer provided in the kit. 5 μL anti-cAMP cryptate was added to all wells of the assay plate, and 5 μL cAMP-d2 added to all wells except non-specific binding (NSB) wells, to which conjugate and lysis buffer was added. Plates were incubated at room temperature for one hour and then read on an Envision (Perkin Elmer) using excitation wavelength of 320 nm and emission wavelengths of 620 nm & 665 nm. Data was transformed to % Delta F as described in manufacturer's guidelines and then transformed to percent activation of maximal native agonist response and analysed by 4-parameter logistic fit to determine EC50 values.

EC50 values for the GIP/GLP-1 agonist polypeptides disclosed herein (see Tables 7 to 10) were determined in cAMP assays are shown in Table 11. The results are compared to corresponding results for native GIP, GLP-1, and glucagon. We used unconstrained 4 parameter logistic fit of data, curve mid-point to determine EC50. This was tested on two occasions, not two batches.

TABLE 11
cAMP activity of GIP/GLP-1 agonist polypeptides

| | hGLP1r | hGCGr | hGIPr | hGIPr (C3) | mGLP1r | mGIPr | rGLP1r | rGIPr |
|---|---|---|---|---|---|---|---|---|
| Ident. | STDEV | STDEV | STDEV | STDEV | STDEV | STDEV | STDEV | STDEV |
| GCG | 8.9E−10 | 5.5E−12 | — | | 1.8E−09 | — | 5.4E−10 | — |
| | 2.3E−10 | 1.3E−12 | | | 8.5E−10 | — | 5.6E−10 | — |
| GLP-1 | 2.2E−11 | — | — | | 1.5E−11 | — | 4.7E−12 | — |
| | 4.3E−12 | | | | 1.4E−11 | — | 5.3E−12 | — |
| GIP | — | — | 4.7E−12 | | | | | |
| | | | 1.6E−12 | | | | | |
| IP024 | 9.5E−11 | 1.7E−08 | 7.1E−12 | | 1.1E−09 | 1.8E−09 | 8.3E−11 | 4.7E−11 |
| | 1.4E−11 | 4.9E−09 | 2.6E−12 | | 5.3E−10 | 1.6E−10 | 3.3E−11 | 3.2E−11 |
| IP085 | 1.5E−10 | 7.1E−09 | 9.9E−12 | | 9.5E−10 | 7.2E−10 | 1.0E−10 | 1.5E−10 |
| | 2.8E−11 | 1.7E−09 | 4.1E−12 | | 3.0E−11 | 5.3E−10 | 4.3E−11 | 1.2E−10 |
| IP131 | 1.0E−10 | 2.3E−08 | 9.1E−12 | | 1.0E−09 | 5.1E−09 | 1.0E−10 | 2.3E−10 |
| | 8.2E−12 | 4.5E−09 | 9.5E−13 | | N = 1 | N = 1 | N = 1 | N = 1 |
| IP266 | 1.5E−10 | 1.6E−08 | 1.2E−11 | | | | | |
| | 2.5E−11 | 1.3E−9 | 3.6E−12 | | | | | |
| IP229 | 1.5E−10 | 2.8E−08 | 6.2E−12 | | | | | |
| | 8.7E−11 | 5.1E−09 | 3.3E−12 | | | | | |
| IP244 | 1.8E−10 | — | 4.2E−11 | | 7.9E−10 | 3.6E−09 | 6.6E−11 | 1.7E−10 |
| | 7.8E−11 | | 8.6E−12 | | 1.1E−10 | 4.9E−10 | 2.7E−11 | 1.3E−10 |
| IP253 | 2.6E−10 | 7.8E−08 | 9.3E−12 | | 3.8E−09 | 6.1E−10 | 2.1E−10 | 2.4E−11 |
| | 7.1E−11 | 1.9E−8 | 7.1E−12 | | N = 1 | N = 1 | N = 1 | N = 1 |
| IP124 | 9.1E−11 | — | 9.0E−12 | | 4.2E−10 | 1.3E−07 | 6.2E−11 | 3.1E−10 |
| | 2.2E−11 | | 2.6E−12 | | 6.1E−11 | 1.4E−07 | 1.0E−11 | 2.6E−10 |
| IP254 | 5.3E−10 | 1.6E−07 | 2.6E−11 | | | | | |
| | 8.1E−11 | 3.4E−8 | 1.6E−11 | | | | | |
| IP256 | 4.0E−10 | >4.2E−07 | 3.7E−11 | | | | | |
| | 8.5E−12 | 1.4E−9 | 1.3E−11 | | | | | |
| IP213 | 8.7E−11 | 1.8E−08 | 5.9E−12 | | 8.2E−10 | 2.5E−09 | 8.7E−11 | 5.4E−11 |
| | 9.9E−13 | 2.8E−09 | 1.1E−12 | | 3.0E−10 | 1.1E−10 | 4.6E−11 | 4.1E−11 |
| IP087 | 2.8E−10 | 9.6E−09 | 8.6E−12 | | 1.2E−09 | 5.7E−09 | 1.1E−10 | 1.7E−10 |
| | 9.8E−11 | 4.0E−09 | 6.7E−13 | | 3.9E−10 | 5.8E−09 | 6.0E−11 | 1.7E−10 |
| IP201 | 2.0E−10 | 3.9E−09 | 1.1E−11 | | 6.5E−10 | 4.3E−09 | 9.8E−11 | 2.3E−10 |
| | 7.1E−12 | 1.2E−10 | 1.7E−12 | | N = 1 | N = 1 | N = 1 | N = 1 |
| IP249 | 2.6E−09 | — | 1.9E−11 | | 1.5E−08 | 1.8E−07 | 1.5E−10 | 1.9E−10 |
| | 2.2E−10 | | 2.1E−12 | | 4.2E−10 | 9.9E−09 | 2.7E−11 | 1.2E−10 |
| IP228 | 1.6E−10 | 1.4E−06 | 1.4E−11 | | 6.9E−10 | — | 9.6E−11 | 7.0E−09 |
| | 5.0E−11 | 8.5E−08 | 3.1E−12 | | 5.7E−10 | — | 9.9E−11 | 9.1E−09 |
| IP222 | 1.4E−10 | 5.9E−07 | 1.5E−11 | | 4.6E−10 | 1.6E−07 | 5.8E−11 | 7.2E−10 |
| | 8.8E−11 | 2.6E−07 | 7.2E−12 | | 5.4E−11 | 1.3E−07 | 5.4E−12 | 1.4E−10 |
| IP255 | 2.2E−10 | >4.6E−06 | 7.0E−11 | | | | | |
| | 3.0E−11 | 2.4E−6 | 2.4E−11 | | | | | |
| IP088 | 7.6E−11 | 4.6E−07 | 7.8E−12 | | 3.1E−10 | 8.6E−08 | 3.3E−11 | 1.0E−10 |
| | 1.5E−11 | 9.8E−08 | 3.0E−12 | | 5.4E−11 | 7.2E−08 | 7.4E−12 | 7.4E−12 |
| IP147 | 6.7E−11 | 1.3E−07 | 3.7E−12 | | 8.5E−09 | — | 1.1E−09 | 6.8E−09 |
| | 7.9E−12 | 2.1E−08 | 1.4E−14 | | 7.3E−09 | — | 1.2E−09 | 9.6E−09 |
| IP149 | 7.0E−11 | 2.0E−10 | 8.1E−12 | | | | | |
| | 9.9E−13 | 7.8E−12 | 3.7E−13 | | | | | |
| IP234 | 7.4E−10 | >1.5E−05 | 4.3E−11 | | | | | |
| | 1.4E−10 | 6.9E−06 | 9.9E−12 | | | | | |
| IP235 | 2.3E−10 | >9.9E−06 | 2.1E−11 | | 6.0E−10 | 2.6E−06 | 3.7E−11 | 6.5E−10 |
| | 4.6E−11 | 9.3E−07 | 8.8E−12 | | 1.7E−10 | 2.1E−06 | 2.2E−11 | 4.5E−11 |
| IP236 | 1.5E−10 | >5.5E−06 | 1.6E−11 | | 3.0E−10 | 4.6E−07 | 2.4E−11 | 2.1E−10 |
| | 8.2E−11 | 3.5E−06 | 1.8E−12 | | 1.5E−10 | 2.9E−07 | 1.5E−11 | 2.1E−11 |
| IP237 | 9.5E−11 | >1.8E−06 | 1.2E−11 | | 2.9E−10 | 1.7E−07 | 2.3E−11 | 1.4E−10 |
| | 6.9E−12 | 4.0E−07 | 1.6E−12 | | 7.1E−11 | 2.5E−08 | 6.2E−12 | 3.0E−11 |
| IP239 | 8.8E−11 | >9.9E−07 | 8.0E−12 | | | | | |
| | 3.9E−11 | 1.5E−07 | 4.1E−13 | | | | | |
| IP210 | 1.8E−10 | — | 1.9E−11 | | | | | |
| | 1.0E−10 | | 5.6E−12 | | | | | |
| IP211 | 2.3E−10 | — | 5.0E−10 | | | | | |
| | 1.3E−10 | | 3.0E−10 | | | | | |
| IP245 | 4.6E−10 | — | 1.5E−10 | | 4.8E−09 | 1.9E−07 | 4.0E−10 | 4.8E−08 |
| | 2.0E−10 | | 3.3E−11 | | 2.4E−09 | na | 3.4E−10 | 2.8E−08 |
| IP246 | 6.5E−10 | >1.4E−06 | 2.2E−09 | | 3.2E−09 | 2.6E−07 | 7.2E−11 | 1.0E−08 |
| | 8.8E−11 | 4.2E−08 | 9.0E−10 | | 4.0E−10 | 1.4E−08 | 4.7E−11 | 5.9E−09 |
| IP247 | 6.3E−08 | >4.2E−07 | 1.9E−11 | | 2.8E−07 | 2.3E−09 | 1.1E−08 | 3.0E−11 |
| | 5.5E−09 | 1.8E−08 | 8.5E−12 | | 1.4E−07 | 1.6E−09 | 4.2E−09 | 2.5E−11 |
| IP243 | 2.5E−10 | >2.1E−06 | 1.0E−11 | | 3.6E−10 | 3.2E−08 | 2.4E−11 | 9.1E−11 |
| | 1.1E−11 | 1.9E−07 | 3.9E−13 | | 1.7E−10 | 7.3E−09 | 1.1E−11 | 2.0E−11 |
| IP212 | 8.7E−11 | >4.0E−06 | 8.1E−12 | | 3.5E−10 | 1.9E−07 | 3.4E−11 | 1.2E−10 |
| | 2.8E−12 | 7.6E−07 | 1.4E−12 | | 3.8E−11 | 1.2E−07 | 6.8E−12 | 1.6E−11 |
| IP252 | 1.9E−10 | >3.2E−06 | 2.4E−11 | | | | | |
| | 2.3E−11 | 3.9E−7 | 8.0E−12 | | | | | |
| IP257 | 7.2E−09 | — | 1.8E−07 | | 4.6E−08 | 4.3E−06 | 1.3E−09 | 1.9E−07 |
| | 3.1E−10 | | 6.8E−08 | | N = 1 | N = 1 | N = 1 | N = 1 |
| IP258 | 4.2E−10 | >1.9E−05 | 3.9E−11 | | 2.3E−09 | 8.7E−07 | 1.2E−10 | 1.5E−09 |
| | 4.2E−11 | | 2.4E−12 | | N = 1 | N = 1 | N = 1 | N = 1 |
| IP259 | 1.0E−10 | >4.8E−07 | 2.7E−11 | | 3.1E−10 | 1.2E−08 | 2.9E−11 | 3.3E−11 |
| | 1.8E−11 | 3.1E−08 | 6.1E−12 | | N = 1 | N = 1 | N = 1 | N = 1 |
| IP260 | 9.1E−10 | >1.6E−05 | 3.3E−11 | | 5.3E−09 | 2.4E−06 | 2.5E−10 | 4.5E−10 |
| | 3.3E−10 | | 1.7E−12 | | N = 1 | N = 1 | N = 1 | N = 1 |
| IP261 | 1.4E−10 | >2.4E−06 | 3.0E−11 | | 3.4E−10 | 2.3E−07 | 2.8E−11 | 2.7E−10 |
| | 4.2E−12 | 1.0E−06 | 5.2E−12 | | N = 1 | N = 1 | N = 1 | N = 1 |
| IP262 | 2.1E−10 | 6.3E−08 | 1.0E−11 | | 5.3E−10 | 3.0E−09 | 3.6E−11 | 1.9E−11 |
| | 8.5E−11 | 7.1E−09 | 7.7E−12 | | N = 1 | N = 1 | N = 1 | N = 1 |
| IP263 | 1.4E−10 | >1.8E−05 | 2.7E−11 | | 9.6E−10 | 7.0E−06 | 4.2E−11 | 1.1E−09 |
| | 3.5E−12 | N = 2 | 5.0E−12 | | N = 1 | N = 1 | N = 1 | N = 1 |
| IP264 | 1.0E−10 | >9.8E−06 | 1.7E−11 | | 3.0E−10 | 9.7E−08 | 2.6E−11 | 9.4E−11 |
| | 2.9E−12 | N = 2 | 1.7E−12 | | N = 1 | N = 1 | N = 1 | N = 1 |
| IP265 | 1.0E−10 | >1.3E−05 | 1.5E−11 | | | | | |
| | 2.5E−11 | N = 2 | 1.3E−12 | | | | | |
| IP172 | 5.7E−11 | >1.5E−05 | 1.6E−08 | | | | | |
| | 2.1E−13 | N = 2 | 1.2E−09 | | | | | |
| IP198 | 4.2E−11 | — | 6.3E−09 | | | | | |
| | 6.2E−12 | | 1.3E−10 | | | | | |
| IP197 | 7.8E−10 | — | 1.8E−10 | | | | | |
| | 2.0E−11 | | 2.3E−11 | | | | | |
| IP170 | 1.2E−10 | — | 7.9E−11 | | 8.5E−10 | 3.4E−08 | 6.0E−11 | 2.3E−09 |
| | 1.3E−11 | | 3.7E−12 | | 2.5E−10 | 2.7E−09 | 3.8E−11 | 1.2E−09 |
| IP209 | 2.3E−10 | — | 5.8E−11 | | 8.4E−10 | 4.9E−09 | 7.5E−11 | 2.2E−10 |
| | 1.0E−10 | | 1.2E−11 | | 1.5E−10 | 1.2E−09 | 3.7E−11 | 7.2E−11 |
| IP226 | 2.0E−10 | — | 2.5E−11 | | 1.0E−09 | 7.5E−09 | 6.1E−11 | 2.7E−10 |
| | 2.8E−12 | | 1.5E−12 | | 2.9E−10 | 2.9E−09 | 4.6E−12 | 1.2E−10 |
| IP250 | 1.1E−09 | — | 1.1E−10 | | 2.2E−08 | 3.5E−08 | 2.2E−09 | 5.3E−09 |
| | 4.9E−11 | | 3.1E−11 | | 1.3E−09 | 1.1E−08 | 9.5E−10 | 3.5E−09 |
| IP223 | 1.6E−08 | — | 2.5E−09 | | 8.0E−08 | — | 1.8E−08 | 7.2E−08 |
| | 4.7E−09 | N = 2 | 8.8E−10 | | 2.6E−08 | — | 9.9E−10 | 1.6E−08 |
| IP227 | 4.8E−08 | — | 2.9E−08 | | | | | |
| | 1.3E−08 | N = 2 | 4.4E−09 | | | | | |
| IP224 | 5.7E−10 | — | 3.5E−10 | | | | | |
| | 3.6E−10 | N = 2 | 1.4E−10 | | | | | |
| IP172 | 5.7E−11 | — | 1.6E−08 | | | | | |
| | 2.1E−13 | N = 2 | 1.2E−09 | | | | | |
| IP171 | 5.3E−10 | — | 3.9E−10 | | | | | |
| | 3.0E−11 | N = 2 | 7.1E−13 | | | | | |
| IP176 | 6.0E−10 | — | 4.2E−10 | | | | | |
| | 2.4E−11 | N = 2 | 1.0E−10 | | | | | |
| IP206 | 8.4E−11 | — | — | | | | | |
| | 1.6E−11 | N = 2 | N = 2 | | | | | |
| IP214 | 7.5E−11 | — | — | | | | | |
| | 2.7E−11 | N = 2 | N = 2 | | | | | |
| IP215 | 5.0E−11 | — | — | | | | | |
| | 2.5E−11 | N = 2 | N = 2 | | | | | |
| IP175 | 5.1E−11 | — | — | | | | | |
| | 3.2E−12 | N = 2 | N = 2 | | | | | |
| IP208 | 1.6E−09 | — | 1.7E−08 | | | | | |
| | 1.1E−09 | N = 2 | 8.6E−09 | | | | | |
| IP204 | 8.5E−07 | — | 1.2E−10 | | | | | |
| | 4.2E−07 | N = 2 | 3.0E−11 | | | | | |
| IP199 | 6.9E−11 | — | — | | | | | |
| | 3.0E−11 | N = 2 | N = 2 | | | | | |
| IP173 | 2.3E−10 | — | 1.2E−09 | | | | | |
| | 2.1E−12 | N = 2 | 7.8E−11 | | | | | |
| IP150 | 3.3E−11 | — | 1.6E−08 | | | | | |
| | 9.1E−12 | N = 2 | 1.7E−09 | | | | | |
| IP174 | 3.2E−11 | — | 4.6E−07 | | | | | |
| | 9.2E−13 | N = 2 | 2.9E−08 | | | | | |
| IP205 | 1.0E−10 | — | 1.2E−09 | | | | | |
| | 3.3E−11 | N = 2 | 1.3E−10 | | | | | |
| IP127 | 8.7E−11 | — | 1.2E−10 | | 6.0E−10 | — | 4.7E−11 | 1.7E−08 |
| | 2.4E−11 | N = 2 | 3.3E−11 | | 8.1E−11 | — | 1.0E−11 | 3.6E−09 |
| IP200 | 1.2E−10 | 1.1E−10 | 1.8E−11 | | | | | |
| | 2.1E−11 | 4.9E−12 | 2.6E−12 | | | | | |
| IP015 | 4.1E−11 | — | 2.7E−08 | | | | | |
| Batch 1 | 6.2E−12 | N = 2 | 3.2E−09 | | | | | |
| IP015 | 2.3E−11 | — | — | | 1.3E−10 | — | 1.4E−11 | — |
| Batch 2 | 4.9E−12 | N = 2 | N = 2 | | 1.1E−10 | — | 1.1E−11 | — |
| IP015 | 3.8E−11 | — | — | | | | | |
| Batch 3 | 1.4E−11 | N = 2 | N = 2 | | | | | |
| IP231 | 6.7E−11 | — | — | | | | | |
| Batch 1 | 5.5E−11 | N = 2 | N = 2 | | | | | |
| IP233 | 3.6E−11 | — | — | | | | | |
| Batch 1 | 1.1E−11 | N = 2 | N = 2 | | | | | |
| IP132 | 4.0E−11 | — | 8.1E−08 | | | | | |
| Batch 2 | 4.7E−12 | N = 2 | 1.6E−09 | | | | | |
| IP146 | 4.9E−11 | 6.5E−07 | 8.3E−08 | | | | | |
| Batch 1 | | | | | | | | |
| IP187 | 3.7E−11 | — | 5.4E−07 | | | | | |
| Batch 1 | 1.2E−12 | N = 2 | 3.5E−08 | | | | | |
| IP191 | 5.7E−11 | — | — | | | | | |
| Batch 1 | 2.3E−11 | N = 2 | N = 2 | | | | | |
| IP192 | 4.3E−11 | — | — | | | | | |
| Batch 1 | 2.4E−12 | N = 2 | N = 2 | | | | | |
| IP193 | 6.4E−11 | 7.8E−07 | 4.5E−07 | | | | | |
| Batch 1 | 1.9E−12 | 1.6E−07 | 6.3E−07 | | | | | |
| IP194 | 6.1E−11 | — | — | | | | | |
| Batch 1 | 1.8E−11 | N = 2 | N = 2 | | | | | |
| IP195 | 6.1E−11 | — | — | | | | | |
| Batch 1 | 1.9E−11 | N = 2 | N = 2 | | | | | |
| IP196 | 7.3E−11 | — | — | | | | | |
| Batch 1 | 2.4E−11 | N = 2 | N = 2 | | | | | |
| IP203 | 4.2E−11 | >6.5E−07 | 1.1E−07 | | | | | |
| Batch 1 | 5.8E−12 | 9.8E−08 | 4.5E−08 | | | | | |
| IP230 | 5.6E−11 | — | >1.5E−05 | | | | | |
| Batch 1 | 3.9E−11 | N = 2 | 6.5E−06 | | | | | |
| IP238 | 5.8E−11 | — | 3.1E−07 | | | | | |
| Batch 1 | 1.9E−11 | N = 2 | 4.9E−08 | | | | | |
| IP122 | 4.4E−11 | — | 1.4E−07 | | | | | |
| Batch 1 | 3.1E−12 | N = 2 | 1.4E−08 | | | | | |
| IP122 | 4.7E−11 | — | 1.3E−07 | | | | | |
| Batch 2 | 1.6E−11 | N = 2 | 2.1E−08 | | | | | |
| IP122 | 8.7E−11 | — | 1.4E−07 | | | | | |
| Batch 3 | 2.3E−11 | N = 2 | 3.6E−08 | | | | | |
| IP018 | — | — | 2.6E−12 | | — | 2.2E−11 | — | 7.6E−12 |
| Batch 1 | N = 2 | N = 2 | 7.8E−13 | | — | 2.5E−11 | — | 2.8E−12 |
| IP018 | — | — | 5.1E−12 | | | | | |
| Batch 2 | N = 2 | N = 2 | 2.6E−12 | | | | | |
| IP188 | 5.0E−07 | 3.5E−07 | 3.8E−12 | | | | | |
| Batch 1 | 3.4E−08 | 6.2E−08 | 7.0E−13 | | | | | |
| IP207 | — | — | 8.5E−12 | | | | | |
| Batch 1 | N = 2 | N = 2 | 1.5E−12 | | | | | |
| IP018 | >0.000000317 | >0.000000317 | | 8.0E−10 | | | | |
| | N = 2 | N = 2 | | 6.4E−10 | | | | |
| IP118 | 1.5E−11 | >0.000000188 | | >0.000000188 | | | | |
| | 1.6E−12 | N + 2 | | N = 2 | | | | |
| IP018 | 4.2E−06 | 4.8E−07 | 9.3E−12 | | | | | |
| Batch 2 | 5.9E−06 | 6.3E−07 | 2.7E−12 | | | | | |
| IP018 | >0.00000635 | 7.2E−07 | 6.4E−12 | | | | | |
| Batch 3 | N = 2 | 1.2E−07 | 5.7E−14 | | | | | |
| IP024 | 1.6E−10 | 2.5E−08 | 1.0E−11 | | | | | |
| Batch 2 | 5.4E−11 | 4.6E−09 | 1.2E−12 | | | | | |
| IP025 | 1.4E−10 | 4.1E−11 | 1.0E−11 | | 1.4E−09 | 3.2E−09 | 6.4E−11 | 3.9E−11 |
| Batch 2 | 9.9E−12 | 3.0E−12 | 1.4E−12 | | 1.5E−10 | 6.4E−11 | 1.5E−11 | 2.5E−12 |
| IP085 | 1.0E−10 | 3.6E−09 | 6.5E−12 | | 5.8E−10 | 2.1E−10 | 2.3E−11 | 3.1E−11 |
| Batch 4 | 2.4E−11 | 2.3E−10 | 3.6E−12 | | 1.6E−11 | 3.4E−11 | 9.2E−12 | 1.9E−12 |
| IP148 | 9.3E−11 | 1.3E−07 | 8.7E−12 | | 3.3E−10 | 1.1E−07 | 1.8E−11 | 1.4E−10 |
| Batch 2 | 1.9E−11 | 2.8E−09 | 3.2E−12 | | 7.1E−11 | 2.7E−08 | 3.5E−13 | 1.5E−11 |
| IP149 | 1.2E−10 | 2.3E−10 | 1.2E−11 | | 3.7E−10 | 5.3E−08 | 3.1E−11 | 1.9E−10 |
| Batch 2 | 5.2E−11 | 9.8E−11 | 8.0E−12 | | 8.6E−11 | 1.6E−08 | 1.1E−12 | 3.5E−12 |
| IP169 | 6.3E−11 | 5.4E−09 | 5.5E−11 | | 1.7E−10 | 3.1E−07 | 2.3E−11 | 3.2E−09 |
| Batch 2 | 3.3E−11 | 1.8E−09 | 3.6E−11 | | 2.2E−11 | 4.3E−08 | 4.9E−13 | 3.7E−10 |
| IP200 | 7.7E−11 | 8.3E−11 | 1.2E−11 | | 3.4E−10 | 1.6E−07 | 2.8E−11 | 2.5E−10 |
| Batch 2 | 2.8E−11 | 2.9E−11 | 3.8E−12 | | 5.4E−11 | 5.5E−08 | 4.1E−12 | 6.6E−11 |
| IP201 | 5.6E−10 | 1.1E−08 | 3.4E−11 | | 4.1E−10 | 1.6E−09 | 2.8E−11 | 1.9E−11 |
| Batch 2 | 2.6E−11 | 4.2E−10 | 5.1E−12 | | N = 1 | N = 1 | N = 1 | N = 1 |
| IP207 | 4.1E−06 | 9.0E−07 | 7.8E−12 | | | | | |
| | 5.8E−06 | 1.2E−06 | 1.6E−12 | | | | | |
| IP243 | 2.1E−10 | 1.4E−06 | 6.9E−12 | | | | | |
| Batch 2 | N = 1 | N = 1 | N = 1 | | | | | |
| IP243 | 1.7E−10 | 1.4E−06 | 7.2E−12 | | | | | |
| Batch 3 | N = 1 | N = 1 | N = 1 | | | | | |
| IP266 | 1.5E−10 | 1.6E−08 | 1.2E−11 | | 7.6E−10 | 3.0E−10 | 3.0E−10 | 3.8E−11 |
| | 2.5E−11 | 1.3E−09 | 3.6E−12 | | 4.0E−10 | 1.6E−11 | 1.5E−11 | 4.9E−12 |
| IP267 | 1.5E−09 | 5.8E−06 | 1.2E−10 | | 1.0E−08 | >0.000000278 | 3.0E−10 | 8.1E−10 |
| | 4.5E−10 | 1.8E−06 | 6.2E−11 | | 3.8E−09 | N = 2 | 1.5E−10 | 2.6E−10 |
| IP268 | 1.2E−10 | 2.9E−06 | 1.0E−11 | 3.8E−09 | 2.8E−10 | 2.1E−07 | 2.6E−11 | 1.5E−10 |
| | 9.2E−12 | 4.0E−07 | 4.9E−13 | 1.5E−09 | 2.7E−11 | 3.1E−08 | 7.1E−13 | 7.1E−13 |
| IP272 | 5.3E−10 | 1.7E−06 | 1.1E−11 | | 2.3E−09 | 1.1E−08 | 8.5E−11 | 4.8E−11 |
| | 1.0E−10 | 8.2E−07 | 4.2E−13 | | 4.2E−10 | 1.8E−09 | 3.0E−11 | 8.7E−12 |
| IP273 | 1.0E−10 | 6.7E−07 | 8.4E−12 | | 1.9E−10 | 8.5E−08 | 2.0E−11 | 9.0E−11 |
| | 5.2E−11 | 3.3E−07 | 4.3E−12 | | 2.3E−11 | 2.2E−08 | 1.5E−12 | 2.9E−12 |
| IP274 | 3.6E−10 | 3.8E−08 | 1.7E−11 | | 1.2E−09 | 1.0E−09 | 5.7E−11 | 2.8E−11 |
| | 6.4E−11 | 5.4E−09 | 1.4E−12 | | 1.4E−10 | 1.2E−10 | 9.9E−12 | 2.0E−12 |
| IP274 | 2.0E−10 | 3.0E−08 | 1.4E−11 | | | | | |
| Batch 2 | N = 1 | N = 1 | N = 1 | | | | | |
| IP275 | 9.2E−10 | 2.3E−07 | 8.7E−12 | | 1.7E−09 | 2.6E−10 | 6.2E−11 | 1.4E−11 |
| | 4.3E−10 | 1.3E−07 | 3.1E−12 | | 2.8E−10 | 2.3E−11 | 1.1E−11 | 2.1E−13 |
| IP276 | 1.6E−10 | 1.5E−06 | 1.3E−11 | | 3.4E−10 | 5.1E−07 | 2.8E−11 | 2.6E−10 |
| | 6.0E−11 | 7.1E−07 | 3.5E−13 | | 0.0E+00 | 1.0E−07 | 6.4E−13 | 1.4E−11 |
| IP278 | 1.3E−10 | 7.8E−08 | 1.1E−11 | | 3.2E−10 | 8.5E−08 | 2.6E−11 | 1.3E−10 |
| | 6.1E−11 | 2.1E−08 | 5.2E−12 | | 6.4E−11 | 4.3E−08 | 5.4E−12 | 3.6E−11 |
| IP279 | 1.0E−10 | 1.7E−07 | 1.0E−11 | | 2.2E−10 | 4.3E−08 | 2.4E−11 | 9.7E−11 |
| | 5.5E−11 | 4.5E−08 | 6.2E−12 | | 6.9E−11 | 1.9E−08 | 8.4E−12 | 2.4E−11 |
| IP280 | 7.7E−11 | 5.2E−07 | 5.6E−12 | | 2.1E−10 | 8.8E−09 | 2.7E−11 | 4.6E−11 |
| | 2.5E−11 | 3.0E−08 | 4.0E−12 | | 3.5E−11 | 2.8E−09 | 2.0E−12 | 2.5E−12 |
| IP280 | 5.8E−11 | 2.7E−07 | 4.5E−12 | | | | | |
| Batch 2 | N = 1 | N = 1 | N = 1 | | | | | |
| IP280 | 8.0E−11 | 2.8E−07 | 6.4E−12 | | | | | |
| Batch 3 | N = 1 | N = 1 | N = 1 | | | | | |
| IP281 | 8.7E−11 | 3.8E−06 | 1.4E−11 | | | | | |
| | 1.9E−11 | 2.3E−07 | 5.3E−12 | | | | | |
| IP283 | 5.6E−10 | >0.000017 | 4.9E−11 | | | | | |
| | 2.6E−10 | | 1.5E−11 | | | | | |
| IP284 | 2.0E−10 | 1.8E−05 | 3.9E−11 | | | | | |
| | 4.5E−11 | 5.5E−06 | 3.2E−11 | | | | | |
| IP285 | 7.6E−11 | 1.6E−06 | 8.7E−12 | | | | | |
| | 2.3E−11 | 7.1E−07 | 4.0E−12 | | | | | |
| IP286 | 7.2E−11 | 9.2E−07 | 8.0E−12 | | | | | |
| | 1.3E−11 | 1.2E−07 | 2.2E−12 | | | | | |
| IP288 | | | 2.3E−06 | | | | | |
| | N = 2 | N = 2 | 3.2E−07 | | | | | |
| IP289 | 3.4E−09 | 2.4E−07 | 1.0E−11 | | 6.8E−09 | 4.4E−10 | 2.3E−10 | 2.0E−11 |
| | 1.3E−10 | 5.7E−09 | 1.2E−12 | | 1.9E−10 | 3.4E−11 | 4.1E−11 | 7.1E−12 |
| IP290 | 1.4E−10 | >0.00000259 | 1.8E−11 | | | | | |
| | 1.5E−11 | | 5.5E−12 | | | | | |
| IP209 | 2.0E−10 | >0.00000627 | 2.8E−11 | | 1.6E−09 | >6.3E−06 | 9.1E−11 | 1.3E−09 |
| Batch 2 | 5.7E−11 | | 6.2E−12 | | 3.9E−10 | N = 2 | 3.9E−11 | 2.1E−10 |
| IP291 | 1.2E−08 | >0.0000106 | 1.2E−10 | | 1.2E−07 | 6.8E−07 | 3.0E−09 | 1.4E−09 |
| | 9.2E−10 | | 4.9E−12 | | 9.2E−09 | 9.3E−08 | 9.5E−10 | 3.2E−10 |
| IP292 | 2.6E−10 | >0.0000136 | 4.3E−11 | | 1.2E−09 | 6.6E−08 | 3.7E−11 | 2.9E−10 |
| | 2.8E−11 | | 4.3E−11 | | 3.1E−10 | 1.9E−08 | 2.5E−11 | 2.5E−11 |
| IP292 | 1.1E−09 | >0.0000149 | 4.6E−11 | | 1.32E−09 | 5.54E−08 | 4.32E−11 | 1.86E−10 |
| Batch 2 | 1.1E−10 | N = 2 | 1.1E−11 | | N = 1 | N = 1 | N = 1 | N = 1 |
| IP293 | 1.6E−09 | >0.000013 | 1.2E−09 | | 1.3E−08 | 7.8E−06 | 4.5E−10 | 1.6E−08 |
| | 1.9E−10 | N = 2 | 2.2E−10 | | 9.3E−09 | 4.3E−06 | 4.4E−10 | 5.6E−09 |
| IP294 | 2.7E−08 | >0.00000681 | 1.3E−09 | | 3.5E−07 | >6.8E−06 | 7.1E−09 | 1.0E−07 |
| | 5.3E−09 | N = 2 | 7.1E−12 | | 1.9E−07 | N = 2 | 6.5E−09 | 9.5E−09 |
| IP295 | 9.1E−11 | 4.1E−08 | 4.3E−12 | | 3.5E−10 | 6.6E−10 | 2.3E−11 | 4.1E−11 |
| | 2.0E−11 | 2.2E−08 | 3.2E−13 | | 4.2E−11 | 9.3E−11 | 8.1E−12 | 2.0E−12 |
| IP295 | 1.4E−10 | 3.2E−08 | 1.2E−11 | | 2.41E−10 | 1.2E−09 | 1.8E−11 | 6.58E−11 |
| Batch 2 | 9.2E−12 | 8.5E−10 | 1.3E−12 | | 6.92965E−11 | 1.62635E−10 | 6.85894E−12 | 3.9103E−11 |
| IP295 | 1.5E−10 | 2.3E−08 | 8.6E−12 | | 3.4E−10 | 5.71E−10 | 2.86E−11 | 2.5E−11 |
| Batch 3 | N = 1 | N = 1 | N = 1 | | N = 1 | N = 1 | N = 1 | N = 1 |
| IP296 | 5.7E−10 | >0.00000112 | 1.8E−11 | | 1.4E−09 | 1.1E−07 | 5.5E−11 | 4.6E−10 |
| | 2.7E−11 | 0.0E+00 | 7.8E−13 | | 2.7E−10 | 7.8E−09 | 1.6E−11 | 4.0E−11 |
| IP296 | 3.9E−10 | >0.000000784 | 2.1E−11 | | 6.0E−10 | 5.5E−08 | 2.8E−11 | 5.4E−10 |
| Batch 2 | 3.9E−11 | 0.0E+00 | 5.7E−13 | | 2.1E−10 | 1.8E−08 | 8.0E−12 | 3.3E−10 |
| IP296 | 3.7E−10 | >0.00000449 | 1.4E−11 | | | | | |
| Batch 3 | 1.2E−10 | 3.1E−06 | 4.7E−12 | | | | | |
| IP296 | 2.9E−10 | 6.7E−06 | 1.1E−11 | | 6.4E−10 | 6.5E−08 | 2.7E−11 | 1.4E−10 |
| Batch 4 | N = 1 | N = 1 | N = 1 | | N = 1 | N = 1 | N = 1 | N = 1 |
| IP297 | 1.9E−10 | 3.1E−08 | 1.5E−11 | | 8.2E−10 | 8.1E−09 | 2.8E−11 | 8.9E−11 |
| | 6.6E−11 | 4.8E−09 | 4.2E−12 | | 1.2E−10 | 1.1E−10 | 9.4E−12 | 4.4E−11 |
| IP299 | >0.00000364 | 5.3E−08 | 2.1E−11 | | >3.64E−06 | 2.1E−07 | 1.9E−08 | 1.1E−10 |
| | N = 2 | 9.9E−09 | 1.3E−12 | | N = 2 | 2.1E−08 | 5.8E−09 | 6.4E−12 |
| IP300 | 2.1E−10 | 2.1E−08 | 1.3E−11 | | 9.4E−10 | 9.0E−10 | 2.7E−11 | 6.9E−11 |
| | 5.6E−11 | 3.9E−09 | 8.5E−13 | | 1.3E−10 | 2.6E−11 | 9.9E−12 | 3.0E−11 |
| IP322 | 2.9E−10 | 2.6E−07 | 2.6E−11 | | 4.1E−10 | 8.3E−09 | 2.5E−11 | 1.1E−10 |
| | 2.1E−12 | 1.7E−08 | 1.7E−11 | | 2.0E−10 | 1.9E−09 | 1.1E−11 | 6.9E−11 |
| IP323 | 2.0E−10 | 1.9E−07 | 1.7E−11 | | 3.4E−10 | 6.5E−08 | 1.9E−11 | 8.2E−11 |
| | 8.5E−11 | 3.4E−08 | 2.7E−12 | | 1.7E−10 | 1.4E−08 | 6.9E−12 | 4.5E−11 |
| IP324 | 4.0E−10 | 4.7E−07 | 3.7E−11 | | 7.2E−10 | 2.4E−07 | 4.0E−11 | 1.4E−10 |
| | 1.3E−10 | 3.0E−07 | 1.4E−11 | | 2.0E−10 | 6.2E−08 | 9.8E−12 | 5.3E−11 |
| IP324 | 1.4E−10 | 1.2E−07 | 1.6E−11 | | 3.91E−10 | 6.75E−08 | 3.63E−11 | 1.41E−10 |
| Batch 2 | 8.0E−11 | 2.6E−08 | 7.8E−12 | | 1.40007E−10 | 1.79605E−08 | 1.13137E−12 | 7.77817E−12 |
| IP325 | 4.6E−10 | >0.000000455 | 3.7E−11 | | 6.4E−10 | 1.7E−07 | 3.5E−11 | 1.3E−10 |
| | 1.2E−10 | N = 2 | 1.5E−11 | | 3.5E−10 | 1.0E−07 | 2.0E−11 | 9.3E−11 |
| IP325 | 1.6E−10 | 1.1E−07 | 1.8E−11 | | 4.88E−10 | 5.72E−08 | 5.19E−11 | 1.54E−10 |
| Batch 2 | 8.3E−11 | 1.4E−09 | 4.9E−12 | | 1.9799E−11 | 1.0748E−08 | 8.41457E−12 | 7.90545E−11 |
| IP326 | 3.7E−10 | 1.5E−08 | 2.7E−11 | | 8.7E−10 | 1.7E−09 | 3.9E−11 | 3.7E−11 |
| | 1.3E−10 | 5.7E−10 | 1.2E−11 | | 1.5E−10 | 1.6E−10 | 9.5E−12 | 4.0E−12 |
| IP327 | 2.5E−10 | 1.0E−08 | 1.6E−11 | | 7.0E−10 | 1.2E−09 | 3.2E−11 | 2.8E−11 |
| | 4.3E−11 | 2.1E−09 | 1.1E−11 | | 3.3E−10 | 2.7E−10 | 1.6E−11 | 8.2E−12 |
| IP328 | 1.2E−09 | 4.3E−08 | 8.5E−11 | | 3.9E−09 | 5.4E−09 | 2.1E−10 | 1.7E−10 |
| | 2.1E−10 | 3.9E−09 | 3.4E−11 | | 1.5E−09 | 8.1E−10 | 1.3E−10 | 7.3E−11 |
| IP328 | 3.3E−10 | 1.3E−08 | 3.3E−11 | | 1.3E−09 | 2.2E−09 | 1.2E−10 | 9.2E−11 |
| Batch 2 | 1.3E−10 | 9.9E−10 | 1.2E−11 | | 5.7E−10 | 7.8E−10 | 3.3E−11 | 5.7E−12 |
| IP339 | 1.8E−10 | 2.3E−08 | 9.8E−12 | | 1.6E−09 | 9.1E−11 | 5.0E−11 | 1.2E−11 |
| | 3.5E−11 | 3.5E−10 | 4.1E−12 | | 1.1E−09 | 1.7E−11 | 6.2E−12 | 2.3E−12 |
| IP341 | 3.1E−11 | 6.9E−06 | 2.0E−07 | | 5.1E−11 | >2.30E−06 | 4.5E−12 | >2.30E−06 |
| | N = 1 | N = 1 | N = 1 | | 4.2E−11 | N = 2 | 1.2E−12 | N = 2 |
| IP354 | 1.4E−09 | >0.00000737 | 2.4E−11 | | 1.3E−08 | 2.7E−07 | 3.6E−09 | 7.4E−10 |
| | 7.3E−10 | N = 2 | 6.7E−12 | | 5.1E−09 | 4.9E−09 | 3.5E−10 | 6.6E−11 |
| IP355 | 8.8E−09 | >0.000000527 | 1.7E−10 | | 2.2E−07 | 1.7E−08 | 2.0E−08 | 5.0E−10 |
| | 2.4E−09 | N = 2 | 3.2E−11 | | #DIV/0! | 4.2E−10 | 1.2E−08 | 8.6E−11 |
| IP357 | 2.7E−10 | 1.1E−07 | 1.4E−11 | | 1.1E−09 | 1.6E−08 | 1.3E−10 | 6.7E−11 |
| | 5.9E−11 | 8.5E−09 | 3.3E−12 | | 5.8E−10 | 5.2E−09 | 6.1E−11 | 1.4E−11 |
| IP358 | 2.4E−10 | 1.4E−07 | 9.4E−12 | | 1.1E−09 | 1.2E−08 | 1.5E−10 | 4.9E−11 |
| | 1.2E−10 | 3.5E−09 | 3.4E−12 | | 5.2E−10 | 2.2E−09 | 1.1E−10 | 2.4E−12 |
| IP359 | 6.1E−11 | 4.3E−08 | 1.1E−11 | | 2.0E−10 | 8.3E−09 | 4.0E−11 | 3.9E−11 |
| | 4.0E−11 | 1.3E−08 | 6.6E−12 | | 9.3E−11 | 2.9E−09 | 2.8E−11 | 2.8E−12 |
| IP360 | 3.1E−10 | 6.3E−08 | 1.6E−11 | 2.5E−09 | 1.2E−09 | 3.6E−10 | 1.5E−10 | 3.3E−11 |
| | 2.9E−10 | 1.8E−08 | 1.2E−11 | 2.4E−09 | 5.0E−10 | 1.4E−10 | 7.7E−11 | 4.4E−12 |
| IP361 | 3.4E−09 | >0.0000062 | 8.2E−11 | | 1.7E−07 | 1.9E−10 | 1.8E−08 | 3.8E−11 |
| | 2.1E−09 | N = 2 | 4.6E−11 | | 2.5E−08 | 6.1E−11 | 1.1E−08 | 1.9E−12 |
| IP362 | 1.7E−09 | >0.00000496 | 6.1E−11 | | 1.1E−08 | 1.1E−09 | 1.1E−09 | 8.3E−11 |
| | 1.1E−09 | N = 2 | 2.6E−11 | | 3.4E−09 | 3.9E−10 | 5.1E−10 | 4.9E−13 |
| IP363 | 9.8E−10 | 5.6E−07 | 5.6E−11 | | 1.1E−08 | 3.1E−10 | 1.4E−09 | 4.5E−11 |
| | 3.4E−10 | 1.8E−08 | 7.1E−12 | | 3.8E−09 | 2.0E−10 | 6.8E−10 | 1.4E−11 |
| IP370 | 6.2E−10 | >0.00000949 | 1.9E−10 | | 1.3E−08 | 4.8E−10 | 3.5E−10 | 1.3E−10 |
| | 4.4E−10 | N = 2 | 3.4E−11 | | 6.7E−09 | 3.2E−10 | 5.4E−11 | 4.5E−11 |
| IP371 | 4.8E−10 | >0.00000656 | 1.1E−11 | | 8.5E−09 | 2.8E−07 | 1.1E−09 | 2.1E−10 |
| | 5.1E−10 | N = 2 | 2.1E−12 | | 3.1E−09 | 2.8E−09 | 1.1E−10 | 1.5E−11 |
| IP372 | 7.1E−10 | >0.00000388 | 5.0E−11 | | 3.3E−08 | 4.5E−07 | 2.9E−09 | 2.7E−08 |
| | 2.9E−10 | N = 2 | 1.9E−12 | | 1.0E−08 | 2.0E−07 | 3.9E−10 | 3.5E−10 |
| IP374 | 1.4E−09 | >0.00000209 | 2.0E−10 | | 1.9E−08 | 1.9E−09 | 8.9E−10 | 4.1E−10 |
| | 8.0E−10 | N = 2 | 1.1E−11 | | 4.9E−09 | 7.9E−10 | 1.3E−11 | 9.5E−11 |
| IP379 | 8.4E−10 | >0.0000132 | 3.8E−12 | | 4.4E−08 | 1.5E−07 | 1.5E−09 | 4.1E−10 |
| | 3.1E−10 | N = 2 | 2.9E−12 | | 2.3E−08 | 5.0E−08 | 8.6E−10 | 2.7E−10 |
| IP380 | 1.1E−10 | 2.5E−08 | 8.4E−12 | | 1.2E−09 | 6.6E−09 | 3.1E−11 | 6.0E−11 |
| | 1.4E−12 | 6.6E−09 | 2.4E−12 | | 5.2E−10 | 2.9E−09 | 3.0E−12 | 3.2E−11 |
| IP381 | 9.1E−11 | 1.8E−08 | 9.9E−12 | | 7.3E−10 | 5.4E−09 | 2.7E−11 | 4.5E−11 |
| | 5.0E−12 | 1.1E−09 | 2.7E−12 | | 2.8E−10 | 1.9E−09 | 7.8E−13 | 1.7E−11 |
| IP382 | 1.5E−09 | 1.2E−07 | 1.9E−10 | | 1.6E−08 | 6.5E−09 | 6.8E−10 | 9.2E−10 |
| | 0.0E+00 | 7.1E−09 | 1.6E−11 | | 5.7E−10 | 9.3E−10 | 2.2E−10 | 3.1E−10 |
| IP383 | 1.9E−10 | >0.000001606 | | | 2.01E−08 | 4.67E−08 | 1.3E−10 | 9.76E−10 |
| | 5.7E−11 | 1.9E−06 | | | 2.12132E−09 | 3.87495E−08 | 1.48492E−11 | 4.87904E−11 |
| IP384 | 9.1E−11 | 2.6E−08 | 6.2E−12 | | 9.9E−10 | 4.1E−09 | 2.4E−11 | 4.3E−11 |
| | 4.7E−11 | 1.2E−08 | 2.4E−12 | | 4.2E−10 | 9.7E−10 | 4.9E−13 | 2.8E−11 |
| IP390 | 1.3E−10 | >0.00000021835 | 8.8E−12 | | 2.0E−09 | 3.6E−09 | 2.4E−11 | 2.3E−10 |
| | 3.0E−11 | 2.5E−07 | 1.0E−12 | | N = 1 | 8.7E−10 | 9.9E−12 | 1.1E−10 |
| IP400 | 2.9E−10 | 1.3E−06 | 2.0E−12 | | 3.1E−08 | 3.8E−07 | 1.9E−10 | 8.7E−11 |
| | 4.9E−12 | 1.5E−06 | N = 1 | | 5.7E−09 | 3.9E−07 | 7.1E−11 | 8.5E−12 |
| IP403 | 9.6E−07 | 9.6E−07 | 1.6E−08 | | >1.74E−06 | 2.1E−08 | 7.3E−08 | 2.0E−11 |
| | 1.1E−06 | 1.1E−06 | 2.2E−08 | | N = 2 | 1.7E−08 | 2.4E−08 | 6.7E−12 |
| IP407 | 2.5E−10 | >0.000000163 | | | | 2.3E−09 | | |
| | 0.0E+00 | N = 2 | | | | N = 1 | | |
| IP410 | >0.00000000799 | >0.00000000799 | | >0.00000000799 | | | | |
| | N = 2 | N = 2 | | N = 2 | | | | |
| IP411 | 4.2E−10 | >0.000000631 | | | | >0.000000631 | | |
| | 0.0E+00 | N = 2 | | | | N = 2 | | |
| IP412 | 7.3E−11 | >0.00000044 | | 1.3E−09 | 1.2E−09 | 4.0E−10 | 2.2E−11 | 1.8E−11 |
| | 0.0E+00 | N = 2 | | 1.2E−09 | 5.2E−10 | 2.9E−10 | 2.3E−11 | 1.4E−11 |
| IP413 | 1.9E−10 | 7.6E−09 | | | 8.9E−09 | 3.7E−09 | 4.8E−11 | >1.13E−07 |
| | 0.0E+00 | 0.0E+00 | | | 3.7E−09 | N = 1 | 3.0E−12 | N = 2 |
| IP415 | 3.8E−11 | 1.8E−11 | | | | 5.6E−09 | | |
| | 1.3E−11 | 3.4E−12 | | | | 2.8E−09 | | |
| IP416 | 1.0E−10 | 3.4E−09 | | | | 1.1E−09 | | |
| | 3.6E−11 | 5.4E−10 | | | | 4.0E−10 | | |
| IP420 | 8.2E−11 | 2.1E−08 | | | | 2.6E−09 | | |
| | 9.7E−12 | 1.6E−09 | | | | 1.5E−09 | | |
| IP298 | 1.8E−10 | >0.0000131 | 1.4E−10 | | 3.6E−10 | 8.0E−08 | 1.5E−11 | 7.5E−09 |
| | 4.2E−11 | 0.0E+00 | 2.1E−11 | | 7.3E−11 | 2.8E−08 | 4.4E−12 | 5.2E−09 |
| IP301 | 3.1E−10 | 1.2E−05 | 1.2E−10 | | 1.9E−09 | 2.2E−08 | 5.8E−11 | 4.7E−09 |
| | 6.5E−11 | 0.0E+00 | 3.3E−11 | | 8.5E−11 | 4.7E−09 | 2.4E−11 | 2.2E−09 |
| IP304 | 1.6E−10 | 2.6E−06 | 1.0E−10 | | 6.9E−10 | 1.6E−08 | 2.4E−11 | 4.6E−09 |
| | 5.7E−12 | 0.0E+00 | 8.1E−12 | | 3.8E−11 | 0.0E+00 | 8.8E−12 | 2.5E−09 |
| IP306 | 1.0E−10 | 2.2E−07 | 1.1E−11 | | 2.5E−10 | 2.5E−10 | 1.5E−11 | 2.6E−11 |
| | 1.5E−11 | 2.1E−08 | 2.9E−12 | | 5.4E−11 | 2.2E−11 | 8.3E−12 | 1.2E−11 |
| IP307 | 1.6E−09 | 4.1E−06 | 9.1E−11 | | 6.5E−09 | 3.1E−08 | 2.1E−10 | 5.2E−09 |
| | 6.4E−11 | 0.0E+00 | 1.1E−12 | | 1.3E−10 | 2.8E−09 | 8.1E−11 | 3.5E−09 |
| IP309 | 7.7E−09 | 9.0E−07 | 8.6E−11 | | 1.6E−08 | 1.5E−08 | 1.7E−09 | 1.3E−09 |
| | 8.9E−10 | 2.6E−07 | 6.4E−12 | | 2.1E−09 | 3.5E−10 | 5.4E−10 | 7.4E−10 |
| IP310 | 3.5E−10 | 3.7E−06 | 1.5E−10 | | 1.4E−09 | 3.1E−08 | 3.8E−11 | 5.2E−09 |
| | 1.5E−11 | 0.0E+00 | 5.8E−11 | | 1.3E−10 | 7.8E−10 | 1.1E−11 | 2.4E−09 |
| IP312 | >0.00000531 | >0.00000531 | 4.8E−11 | | >1.06E−06 | 1.6E−08 | 5.4E−09 | 1.2E−09 |
| | 0.0E+00 | 0.0E+00 | 1.5E−11 | | N = 2 | 7.1E−10 | 1.5E−09 | 5.5E−10 |
| IP315 | >0.00000546 | 2.8E−07 | 2.4E−11 | | >5.46E−06 | 3.6E−10 | 1.3E−08 | 9.6E−11 |
| | 0.0E+00 | 7.0E−08 | 2.3E−12 | | N = 2 | 3.1E−11 | 4.9E−09 | 5.2E−11 |
| IP317 | 1.3E−10 | >0.00000555 | 1.1E−10 | | 3.3E−10 | 8.6E−08 | 1.2E−11 | 5.3E−09 |
| | 1.4E−12 | 0.0E+00 | 6.4E−12 | | 4.2E−12 | 5.1E−08 | 4.8E−12 | 2.5E−09 |
| IP329 | 1.4E−09 | >0.00000143 | 1.1E−11 | | 9.0E−09 | 2.6E−09 | 1.8E−10 | 6.0E−11 |
| | 2.6E−10 | N = 2 | 7.9E−12 | | 5.2E−09 | 9.5E−10 | 1.8E−11 | 9.0E−12 |
| IP330 | 6.6E−10 | 1.1E−06 | 9.0E−12 | | 5.5E−09 | 4.5E−10 | 8.9E−11 | 2.3E−11 |
| | 1.0E−10 | 2.2E−07 | 6.9E−12 | | 2.8E−09 | 1.7E−11 | 2.9E−11 | 5.8E−12 |
| IP331 | 2.6E−09 | >0.00000195 | 1.3E−11 | | 2.2E−08 | 4.0E−09 | 3.1E−10 | 6.0E−11 |
| | 4.5E−10 | N = 2 | 5.5E−12 | | 1.6E−08 | 1.7E−09 | 8.1E−11 | 8.1E−12 |
| IP343 | 9.6E−07 | >0.00000207 | 7.2E−08 | | >2.07E−06 | 8.8E−07 | 3.6E−07 | 1.1E−07 |
| | 7.1E−09 | N = 2 | 3.0E−08 | | N = 2 | 2.6E−07 | 1.3E−07 | 1.4E−09 |
| IP346 | 8.0E−11 | 1.8E−07 | 1.1E−11 | 1.6E−10 | 2.9E−10 | 1.6E−10 | 1.7E−11 | 1.0E−11 |
| | 3.6E−11 | 1.2E−08 | 1.9E−12 | 2.0E−10 | 1.4E−10 | 1.0E−10 | 9.6E−12 | 2.1E−12 |
| IP347 | 7.5E−10 | >0.00000149 | 1.3E−11 | | 2.5E−09 | 8.5E−10 | 8.7E−11 | 2.4E−11 |
| | 1.5E−10 | N = 2 | 7.1E−14 | | 2.5E−10 | 2.2E−10 | 9.8E−12 | 3.7E−12 |
| IP348 | 2.9E−10 | >0.000000907 | 1.1E−11 | | 8.9E−10 | 1.4E−09 | 3.7E−11 | 2.4E−11 |
| | 1.4E−10 | N = 2 | 1.8E−12 | | 3.8E−10 | 9.1E−10 | 2.0E−11 | 6.4E−13 |
| IP349 | 1.6E−10 | 1.2E−06 | 7.9E−12 | | 8.6E−10 | 5.5E−10 | 5.9E−11 | 1.4E−11 |
| | 9.8E−11 | 2.1E−07 | 3.5E−12 | | 4.4E−10 | 3.4E−10 | 3.7E−11 | 3.0E−12 |
| IP350 | 8.6E−10 | >0.00000136 | 1.3E−11 | | 2.7E−09 | 1.3E−09 | 2.1E−10 | 2.4E−11 |
| | 6.0E−10 | N = 2 | 1.7E−12 | | 1.2E−09 | 7.7E−10 | 1.6E−10 | 6.9E−12 |
| IP352 | 1.3E−10 | 6.2E−07 | 6.9E−12 | | 3.6E−09 | 5.2E−10 | 7.6E−12 | 8.9E−12 |
| | 2.3E−11 | #DIV/0! | 8.3E−13 | | N = 1 | 1.6E−10 | 3.0E−12 | 7.8E−13 |
| IP364 | 1.8E−10 | 6.1E−07 | 8.5E−12 | | 6.9E−10 | 5.0E−10 | 5.8E−11 | 2.0E−11 |
| | 3.5E−11 | 8.6E−08 | 2.1E−12 | | 2.4E−10 | 2.0E−10 | 5.9E−12 | 6.9E−12 |
| IP365 | 8.7E−10 | >0.0000107 | 2.0E−11 | | 1.1E−08 | 1.7E−08 | 1.5E−09 | 1.2E−10 |
| | 3.2E−10 | N = 2 | 1.6E−12 | | 6.3E−09 | 7.6E−09 | 7.1E−10 | 3.5E−11 |
| IP366 | 6.1E−09 | >0.0000102 | 1.4E−10 | | 1.0E−07 | 5.6E−11 | 1.4E−08 | 1.6E−11 |
| | 2.3E−09 | N = 2 | 8.5E−12 | | 1.8E−08 | 3.8E−11 | 8.5E−09 | 2.8E−12 |
| IP367 | 4.4E−09 | >0.0000013 | 5.7E−11 | 1.3E−08 | 3.9E−07 | 3.1E−11 | 8.0E−09 | 9.9E−12 |
| | 1.8E−09 | N = 2 | 1.9E−11 | 1.8E−08 | 2.0E−07 | 1.1E−11 | 4.6E−09 | 2.9E−12 |
| IP368 | 2.3E−07 | >0.00000486 | 2.0E−10 | | >4.86E−06 | 1.3E−09 | 3.7E−07 | 2.2E−10 |
| | 0.0E+00 | N = 2 | 0.0E+00 | | N = 2 | 4.9E−10 | 2.1E−07 | 1.8E−11 |
| IP369 | 7.1E−11 | 7.1E−07 | 8.9E−12 | | 2.7E−10 | 6.8E−10 | 2.3E−11 | 2.0E−11 |
| | 2.2E−11 | 1.1E−07 | 3.5E−13 | | 7.6E−11 | 2.4E−10 | 4.4E−12 | 3.5E−13 |
| IP373 | 7.2E−10 | >0.00000679 | 5.4E−12 | | 9.2E−09 | 3.2E−09 | 9.0E−10 | 3.5E−11 |
| | 1.1E−10 | N = 2 | 3.0E−12 | | 3.1E−09 | 1.4E−09 | 1.0E−10 | 3.2E−12 |
| IP375 | 4.0E−09 | >0.0000117 | 7.8E−11 | | 4.9E−08 | 6.5E−10 | 3.9E−09 | 7.0E−11 |
| | 4.4E−09 | N = 2 | 3.9E−12 | | 2.6E−09 | 1.4E−10 | 1.2E−09 | 4.2E−12 |
| IP376 | 2.4E−09 | >0.00000743 | 8.3E−11 | | 2.1E−08 | 5.5E−10 | 1.3E−09 | 1.3E−10 |
| | 2.6E−09 | N = 2 | 5.2E−12 | | 4.7E−09 | 2.6E−10 | 2.1E−11 | 4.0E−11 |
| IP378 | 1.7E−08 | >0.0000022495 | | | 1.0E−07 | 3.1E−09 | 1.8E−09 | 4.0E−10 |
| | 6.2E−09 | 2.6E−06 | | | 5.4E−08 | 2.6E−09 | 2.8E−10 | 1.4E−10 |
| IP385 | 4.7E−10 | >0.000001562 | 1.2E−11 | | 3.0E−08 | 1.8E−08 | 1.6E−10 | 1.1E−09 |
| | 2.0E−10 | 1.8E−06 | 4.6E−12 | | N = 1 | 7.8E−10 | 2.3E−11 | 4.9E−10 |
| IP394 | 5.4E−10 | >0.0000176 | 9.5E−12 | | 1.0E−08 | 1.0E−08 | 2.3E−10 | 6.0E−11 |
| | 8.3E−11 | N = 2 | 2.0E−13 | | 1.6E−09 | 3.5E−09 | 1.7E−10 | 3.4E−11 |
| IP395 | 1.2E−10 | 3.8E−07 | 1.2E−11 | | 3.8E−09 | 3.3E−09 | 1.6E−11 | 6.3E−11 |
| | 3.3E−11 | 4.4E−07 | 3.6E−12 | | N = 1 | 5.8E−10 | 2.3E−12 | 3.6E−11 |
| IP396 | 1.1E−10 | 3.3E−07 | 9.3E−12 | | 1.2E−09 | 2.8E−10 | 5.7E−11 | 1.9E−10 |
| | 5.8E−11 | 1.5E−07 | 3.6E−12 | | 7.1E−10 | 9.7E−11 | 7.9E−12 | 2.3E−10 |
| IP396 | 1.1E−10 | 2.5E−07 | | | 2.5E−09 | 7.8E−10 | 1.0E−11 | 1.4E−11 |
| Batch 2 | 1.1E−11 | 4.9E−08 | | | N = 1 | 1.1E−09 | N = 1 | N = 1 |
| IP401 | 3.5E−10 | 3.1E−06 | 3.5E−09 | | 1.3E−08 | 3.6E−09 | 7.3E−11 | 1.4E−11 |
| | 3.0E−11 | 3.6E−06 | 5.0E−09 | | 1.6E−09 | 2.8E−09 | 2.0E−11 | 1.9E−12 |
| IP404 | >0.0000000172 | >0.0000000172 | | | | 5.0E−11 | | |
| | N = 2 | N+2 | | | | 2.3E−11 | | |
| IP408 | >0.000000166 | >0.000000166 | | | | 3.8E−10 | | |
| | N = 2 | N = 2 | | | | N = 1 | | |
| IP422 | >0.000000059 | >0.000000059 | | 1.8E−09 | | 1.2E−11 | | |
| | N = 2 | N = 2 | | 2.4E−09 | | 4.1E−12 | | |
| IP305 | 2.3E−07 | 1.1E−06 | 2.6E−10 | | >1.13E−06 | 3.2E−09 | 4.7E−08 | 1.7E−09 |
| | 6.6E−08 | 0.0E+00 | 1.3E−10 | | N = 2 | 5.2E−10 | 1.3E−08 | 1.1E−09 |
| IP314 | >0.000000906 | 7.4E−07 | 2.3E−10 | | >9.06E−07 | 1.5E−08 | 4.5E−07 | 8.8E−10 |
| | 0.0E+00 | 2.3E−07 | 5.6E−11 | | N = 2 | 2.5E−09 | 1.1E−07 | 6.0E−10 |
| IP318 | 4.5E−06 | 4.5E−06 | 5.0E−11 | | >4.47E−06 | 3.5E−08 | >4.47E−06 | 1.4E−08 |
| | 0.0E+00 | 0.0E+00 | 3.5E−12 | | N = 2 | 1.5E−08 | N = 2 | 1.0E−08 |
| IP391 | 3.0E−11 | >0.00001067 | 2.5E−08 | | 3.4E−10 | 1.3E−07 | 1.1E−11 | 4.2E−08 |
| | 1.0E−11 | 1.2E−05 | 1.3E−09 | | N = 1 | 1.4E−09 | 1.3E−12 | 1.7E−08 |
| IP421 | 3.0E−09 | >0.000000106 | | | | >0.000000106 | | |
| | 1.7E−10 | N = 2 | | | | N = 2 | | |
| IP423 | >0.0000000342 | >0.0000000342 | | | | 1.2E−08 | | |
| | N = 2 | N = 2 | | | | 1.5E−08 | | |
| IP302 | 2.6E−08 | 1.2E−06 | 1.8E−11 | | 9.3E−08 | 8.3E−11 | 7.0E−09 | 5.5E−11 |
| | 1.8E−09 | 0.0E+00 | 5.1E−12 | | 1.4E−08 | 7.7E−12 | 2.1E−09 | 2.2E−11 |
| IP308 | 1.3E−06 | 1.5E−06 | 2.5E−11 | | >1.52E−06 | 1.7E−10 | 7.0E−07 | 9.0E−11 |
| | 3.1E−07 | 0.0E+00 | 3.6E−12 | | N = 2 | 2.1E−11 | 1.6E−07 | 5.3E−11 |
| IP311 | 6.7E−07 | >0.00000242 | 7.4E−11 | | 1.5E−06 | 9.5E−10 | 6.1E−08 | 4.3E−10 |
| | 2.3E−07 | 0.0E+00 | 2.4E−11 | | 1.1E−06 | 2.3E−10 | 6.3E−08 | 2.7E−10 |
| IP319 | 1.7E−07 | >0.000000445 | 1.4E−08 | | | | | |
| Batch 2 | 8.3E−08 | 0.0E+00 | 6.4E−10 | | | | | |
| IP320 | 1.6E−07 | 3.8E−06 | 9.6E−09 | | 2.5E−06 | >3.93E−06 | 1.3E−07 | 1.3E−07 |
| | 4.1E−08 | 1.3E−07 | 2.8E−09 | | 4.3E−07 | N = 2 | 2.1E−08 | 4.5E−08 |
| IP320 | 2.6E−07 | 3.8E−06 | 4.4E−08 | | | | | |
| Batch 2 | 9.7E−08 | 1.3E−07 | 4.6E−08 | | | | | |
| IP332 | 9.8E−08 | >0.00000125 | 2.6E−11 | | 7.5E−07 | 2.1E−10 | 5.0E−08 | 4.4E−11 |
| | 1.9E−08 | N = 2 | 1.7E−11 | | 2.1E−07 | 8.1E−11 | 7.9E−09 | 1.6E−12 |
| IP333 | >0.00000108 | >0.00000108 | 8.6E−12 | | >1.08E−06 | 1.9E−11 | 4.3E−07 | 1.5E−11 |
| | N = 2 | N = 2 | 3.9E−12 | | N = 2 | 8.6E−12 | 7.9E−08 | 1.4E−13 |
| IP334 | 1.3E−07 | >0.000000455 | 2.9E−11 | | >4.55E−07 | 3.0E−10 | 1.2E−07 | 5.6E−11 |
| | 7.2E−08 | N = 2 | 1.2E−11 | | N = 2 | 1.7E−10 | 3.7E−08 | 3.6E−12 |
| IP342 | 7.5E−08 | >0.00000356 | 4.7E−09 | | 6.7E−07 | 4.3E−08 | 2.3E−08 | 5.1E−09 |
| | 7.1E−09 | N = 2 | 2.5E−09 | | 4.1E−07 | 1.8E−08 | 7.7E−09 | 2.5E−10 |
| IP353 | 2.0E−08 | >0.00000321 | 1.0E−09 | | | | | |
| | 1.0E−08 | N = 2 | 4.2E−11 | | | | | |
| IP321 | 7.2E−08 | 2.4E−08 | 9.6E−12 | | 3.3E−07 | 1.7E−11 | 2.2E−08 | 7.9E−12 |
| | 8.4E−09 | 4.6E−09 | 5.4E−12 | | 7.0E−08 | 5.1E−12 | 9.6E−09 | 2.8E−12 |
| IP335 | >0.00000548 | 2.8E−07 | 5.8E−12 | | >2.74E−06 | 2.0E−11 | >2.74E−06 | 6.7E−12 |
| | N = 2 | 2.4E−08 | 2.1E−12 | | N = 2 | 3.7E−12 | N = 2 | 4.7E−13 |
| IP336 | >0.00000451 | 2.0E−07 | 5.0E−12 | | >2.26E−06 | 1.2E−11 | >2.26E−06 | 4.7E−12 |
| | N = 2 | 2.0E−08 | 3.7E−13 | | N = 2 | 1.6E−12 | N = 2 | 7.1E−15 |
| IP337 | >0.0000023 | 2.0E−07 | 6.2E−12 | | >1.15E−06 | 1.3E−11 | >1.15E−06 | 5.2E−12 |
| | N = 2 | 3.2E−08 | 4.5E−12 | | N = 2 | 4.4E−12 | N = 2 | 4.3E−13 |
| IP338 | >0.00000189 | 1.9E−07 | 5.2E−12 | | >9.45E−07 | 1.2E−11 | >9.45E−07 | 5.0E−12 |
| | N = 2 | 2.2E−08 | 2.6E−12 | | N = 2 | 4.3E−12 | N = 2 | 1.1E−12 |
| IP344 | >0.00000855 | 2.0E−07 | 5.3E−12 | | >8.55E−06 | 1.1E−11 | >8.55E−06 | 6.2E−12 |
| | N = 2 | 1.6E−08 | 3.4E−13 | | N = 2 | 6.4E−13 | N = 2 | 2.5E−12 |
| IP351 | 1.1E−07 | 1.3E−07 | 3.6E−12 | | 8.2E−07 | 9.8E−12 | 2.1E−07 | 6.3E−12 |
| | 4.0E−08 | 1.2E−08 | 1.7E−12 | | 2.6E−07 | 4.0E−12 | 1.4E−07 | 4.1E−13 |
| IP386 | >0.00000913 | 5.4E−07 | 3.4E−12 | | >1.66E−05 | 3.0E−11 | >1.66E−05 | 8.5E−12 |
| | 1.1E−05 | 2.5E−08 | 1.7E−12 | | N = 1 | 1.1E−11 | N = 2 | 4.8E−12 |
| IP387 | 3.7E−07 | 2.2E−07 | 4.5E−12 | | >1.47E−06 | 1.9E−11 | 1.1E−07 | 7.5E−12 |
| | #Div/0! | #DIV/0! | 2.7E−12 | | N = 1 | 8.5E−12 | 2.0E−08 | 4.9E−12 |
| IP388 | >0.0000052745 | 2.7E−07 | 3.0E−12 | | >9.59E−06 | 1.9E−11 | >9.59E−06 | 6.6E−12 |
| | 6.1E−06 | 5.8E−08 | 5.7E−13 | | N = 1 | 4.7E−12 | N = 2 | 3.4E−12 |
| IP389 | 6.5E−08 | >0.000000269 | 2.7E−12 | | 1.3E−06 | 1.7E−11 | 9.9E−09 | 7.2E−12 |
| | 3.4E−08 | 4.4E−08 | 1.3E−13 | | N = 1 | 8.5E−13 | 8.3E−10 | 4.1E−12 |
| IP392 | 2.8E−06 | 5.3E−07 | 2.6E−12 | | >1.71E−05 | 3.1E−11 | 7.2E−07 | 7.6E−12 |
| | #DIV/0! | 3.8E−08 | 2.3E−13 | | N = 1 | 1.3E−11 | 4.9E−08 | 5.7E−12 |
| IP398 | 9.5E−06 | 1.6E−06 | 3.7E−12 | | >1.72E−05 | 1.2E−11 | 1.4E−05 | 5.6E−12 |
| | 1.1E−05 | 1.5E−07 | 1.7E−12 | | N = 1 | 9.9E−13 | 2.8E−07 | 2.2E−13 |
| IP402 | 3.1E−08 | 2.0E−07 | 3.8E−11 | | 8.5E−07 | 3.3E−11 | 1.1E−08 | 6.8E−12 |
| | 4.6E−09 | 1.1E−07 | 4.9E−11 | | 3.5E−07 | 2.5E−11 | 3.8E−09 | 2.2E−12 |
| IP419 | >0.0000000259 | >0.0000000259 | | | | 1.2E−11 | | |
| | N = 2 | N = 2 | | | | 6.1E−12 | | |

Blank fields in human assays means “not active,” except column hGIPr (C3) wherein blank fields mean “not tested”;
Blank fields in rodent assays means “not tested.”
Where no “Batch” number is indicated, Batch “1” was tested.

### Example 3

### In Vitro Characterization of IP088

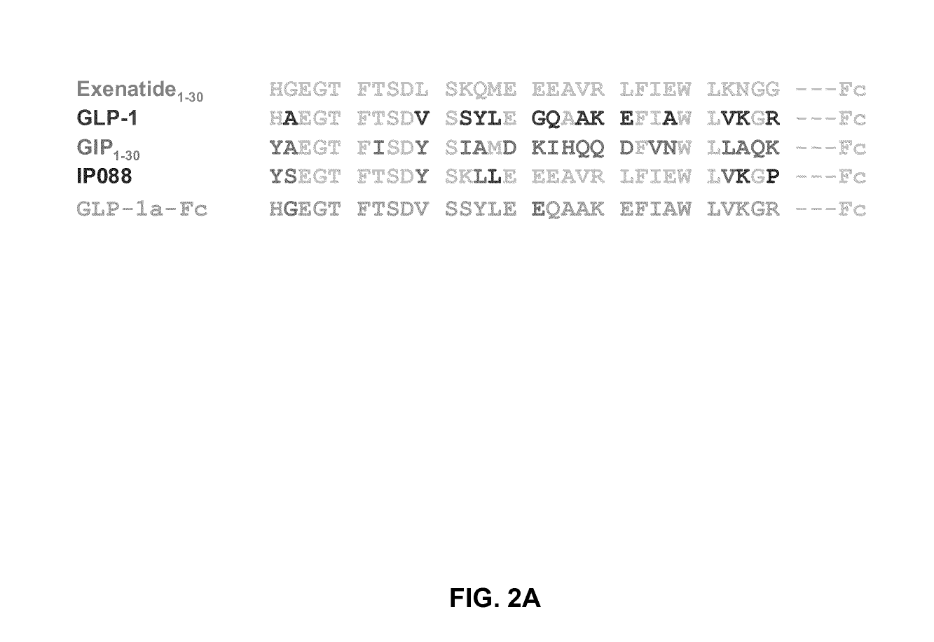

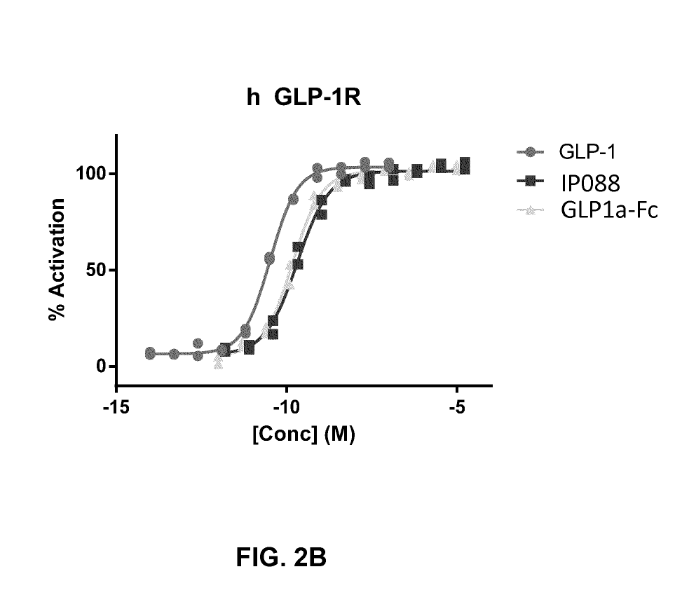

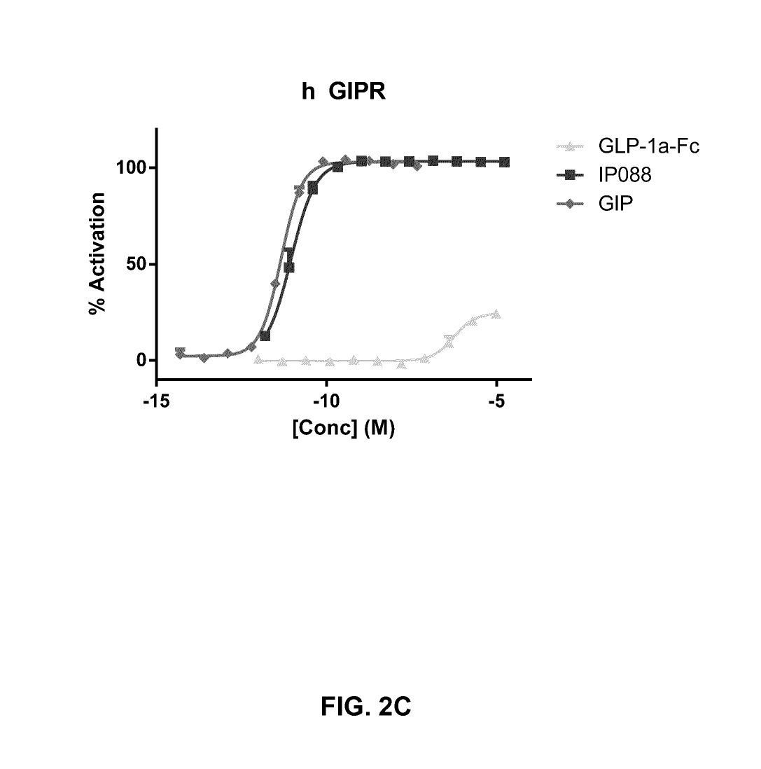

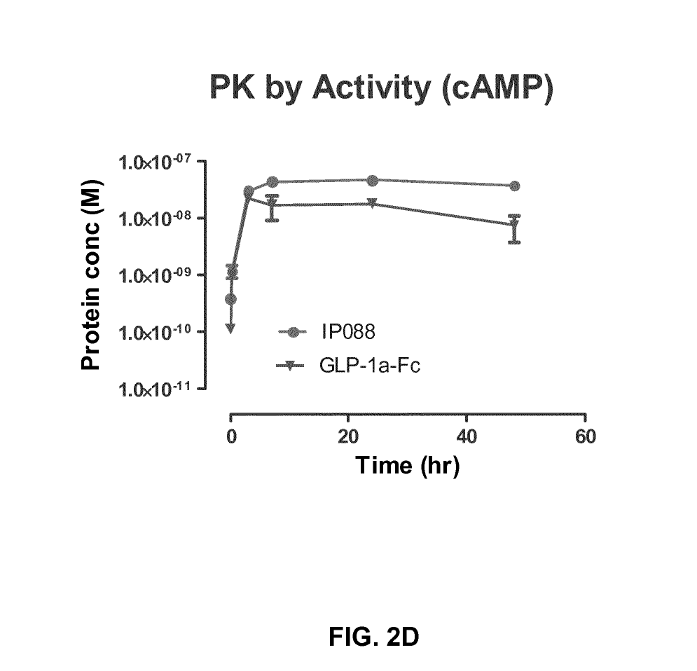

Certain of the GIP/GLP-1 agonist peptides tested were evaluated for further studies. Desirable properties included, without limitation, high potency, e.g., picomolar range EC5Os for both human GIPr and human GLP-1r, EC5Os for the human glucagon receptor (GCGr) of much lower potency, e.g., at least 100-fold, 1000-fold, or more lower potency for GCGr (e.g., in the micromolar range), and, to allow for in vivo studies in rodent models, high potency, e.g., picomolar-to-nanomolar range EC5Os for either mouse or rat GIPr and GLP-1r. The GIP/GLP-1 dual agonist polypeptide IP088 was chosen for further analysis. FIG. 2A shows an alignment if IP088 with Exendin-4, hGLP-1, and hGIP, as well as GLP1a-Fc.

The potency of IP088 on hGLP-1r and hGIPr in comparison with hGLP-1 and hGIP is shown in FIGS. 2B and 2C. Potency curves for the GLP1a-Fc are also shown for comparison.

Preliminary pharmacokinetics in mouse was examined as follows: C57 Bl/6J mice were injected with 1 mg/kg (subcutaneously) with either IP088 or GLP1a-Fc, and serum samples were taken over time (3 animals per time point). The serum samples were then measured for activity at the hGLP-1r by the cAMP assay in HBSS/HEPES 0.1% BSA, as described above. The results are shown in FIG. 2D. The results show that the GIP/GLP-1 agonist polypeptide IP088 is stable, and maintains its activity in the nanomolar range for at least 48 hours in vivo.

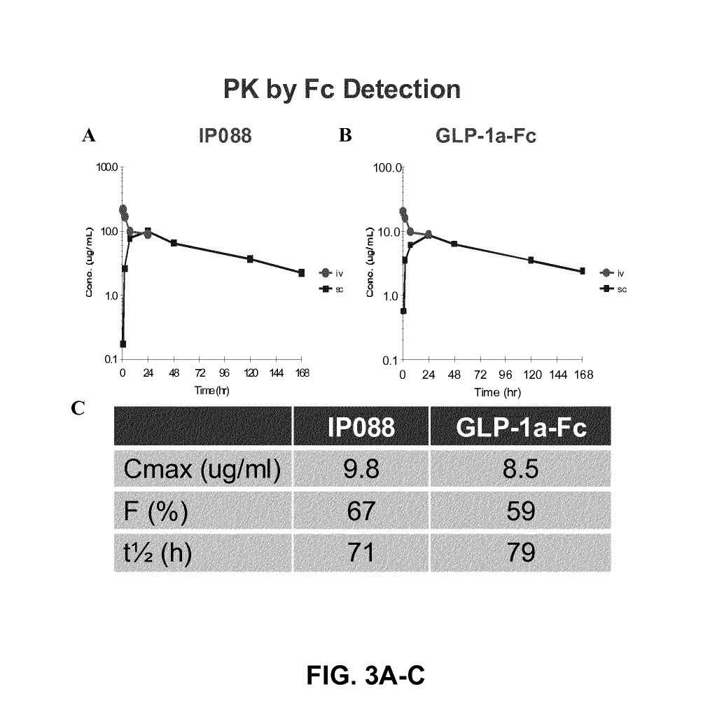

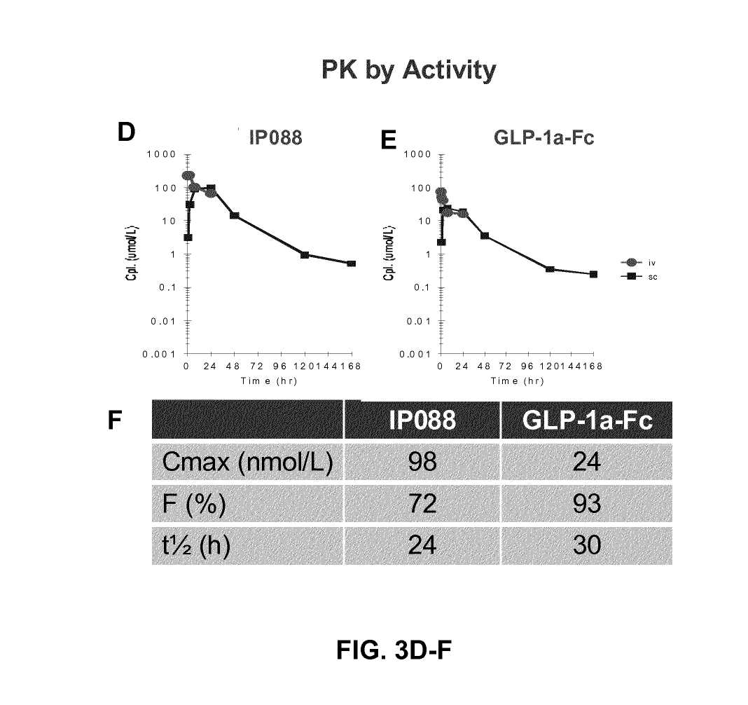

More extended pharmacokinetic assays to determine protein stability and activity were performed as follows. CD male rats (n=3) were injected subcutaneously or intravenously with 1 mg/kg IP088 or GLP1a-Fc. Periodic serum samples were obtained out to 168 hours. The serum samples were subjected to ELISA for protein (Fc) detection by the following method. ELISA plates (Costar 3690) were coated with Mouse anti-human IgG (JCD-10) 1/500 in PBS (final concentration 1 μg/ml). 50 μm of diluted serum was added to each well and the plates were incubated overnight at 4° C. Following washing with PBS-Tween (PBS-T), the plates were blocked with 120 μm 1% BSA in PBS for 1 h at RT with gentle shaking. Following washing with PBS-T, standards, and dilutions of mouse serum samples were added (50 μl/well), and the plate was incubated for 2 h at RT with gentle shaking. Following washing 50 μm biotinylated mouse anti-human IgG (JCD-10) diluted 1/1000 in ELISA incubation buffer (final concentration 0.5 μg/ml) was added to each well and the plates were incubated for 1 h at RT with gentle shaking. Following washing 50 μm of Streptavidin-HRP diluted 1/1000 in ELISA incubation buffer was added to each well, and the plates were incubated for 45 min at RT with gentle shaking. Following a final washing, 50 μl/well of TMB solution (Invitrogen #00-2023) was added to each well and the plates were incubate in the dark until desired blue color appeared (5-10 min). The reaction was stopped by addition of 50 μm of stop solution (1 M HCl). The results are plotted in FIG. 3A and FIG. 3B. IP088 and GLP1a-Fc had equivalent PK profiles, as shown in FIG. 3C.

The serum samples obtained were also tested for potency against hGLP-1r by the cAMP assay as described above in HBSS/HEPES 0.1% BSA. The results are plotted in FIG. 3D and FIG. 3E. IP088 and GLP1a-Fc had similar PK activity profiles, as shown in FIG. 3F.

In order to be able to carry out in vivo studies in rodents, IP088 was tested for potency against the mouse receptors mGIPr and mGLP-1r as well as the rat receptors rGIPr and rGLP-1r, using cells expressing the respective receptors in the cAMP assay described above. The relative potencies expressed as EC5Os are shown in Table 12. IP088 showed high potency across species for GLP-1r and showed high potencies for human and rat GIPr, but showed relatively lower potency for mouse GIPr.

TABLE 12
Relative Potencies of IP088 against Human,
Rat, and Mouse GLP-1 and GIP Receptors

| | | |
|---|---|---|
| Receptor | EC50 for GLP-1r | EC50 for GIPr |

| | | | | |
|---|---|---|---|---|
| Human | 222 | pM | 16 | pM |
| Mouse | 420 | pM | 137 | nM |
| Rat | 38 | pM | 99 | M |

Further potency assays were carried out using the GLP-1 receptor from the cynomolgus monkey, using the cAMP assay described above, the assay was carried out with cells expressing cyno GLP-1r or cyno GCGr using culture medium containing 4.2% cyno serum albumin (Equitech Bio Ltd.). The results are shown in Table 13.

TABLE 13
Potencies of IP088 against Cynomolgus
Monkey GLP-1 and Glucagon Receptors

| | |
|---|---|
| EC50 for cGLP-1r | EC50 for cGCGr |

| | | | | |
|---|---|---|---|---|
| IP088 | 190 | pM | >1 | μM |
| GLP1a-Fc | 11 | pM | >1 | μM |
| liraglutide | 25 | pM | >1 | μM |

| | | | |
|---|---|---|---|
| hGLP-1 | 1.8 | pM | Not tested |

| | | | |
|---|---|---|---|
| hGCG | Not tested | 3.6 | pM |

### Example 3

### Oral Glucose Tolerance Test in Mouse Model

The effect of IP088 on glycemic control in a mouse model was carried out as follows. Twenty-one (21) male C57 male mice (11-12 weeks old) were purchased from Taconic, Denmark and acclimatized for 3 weeks before experimentation. Throughout the acclimatization period the animals are offered ad libitum chow and tap water. Mice were single housed one week prior to the study. On day 1 of the study animals were randomized into 4 groups based on body weight (n=5-6 per group), and were administered the following compounds subcutaneously four hours (IP088, IP040, and GLP1a-Fc) or two hours (liraglutide) prior to each oral glucose tolerance test:

Group 5: IP040 (vehicle)

Group 6: Liraglutide (GLP-1 analog, Novo Nordisk) 0.2 mg/kg S.C.

Group 7: GLP1a-Fc (GLP-1 agonist) 1 mg/kg S.C.

Group 8: IP088 1 mg/kg S.C.

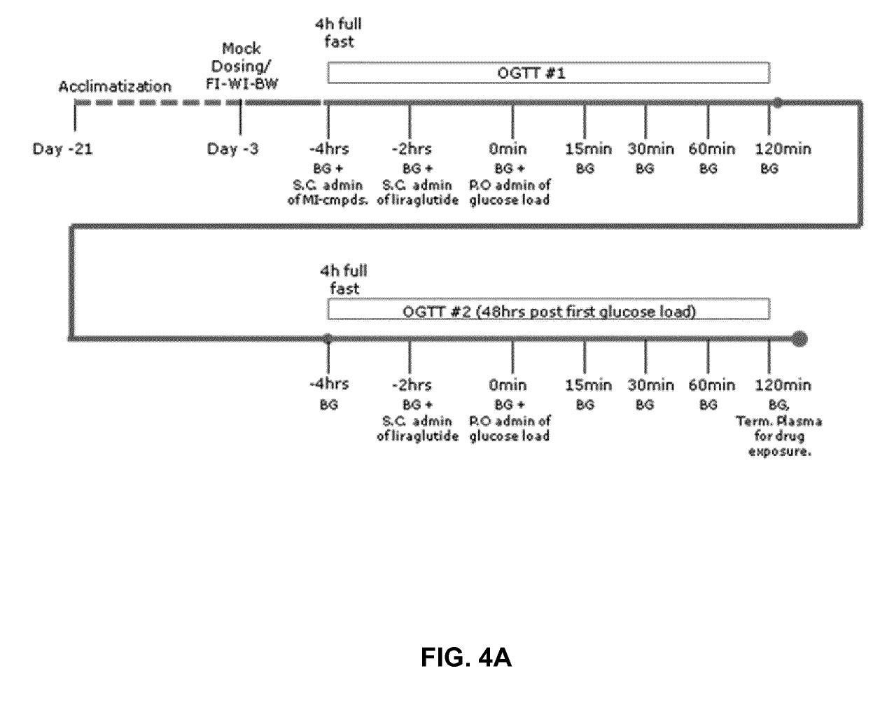

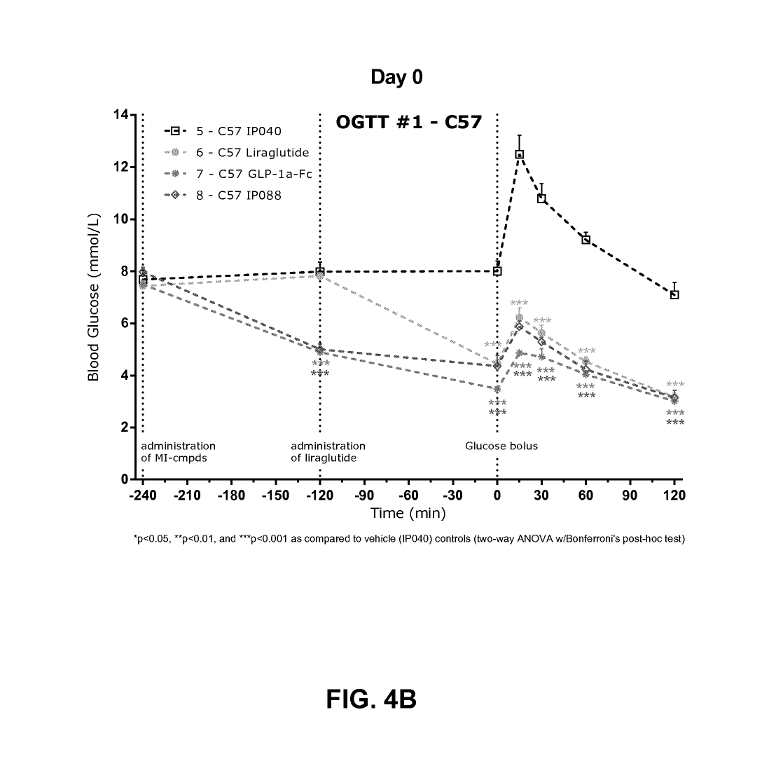

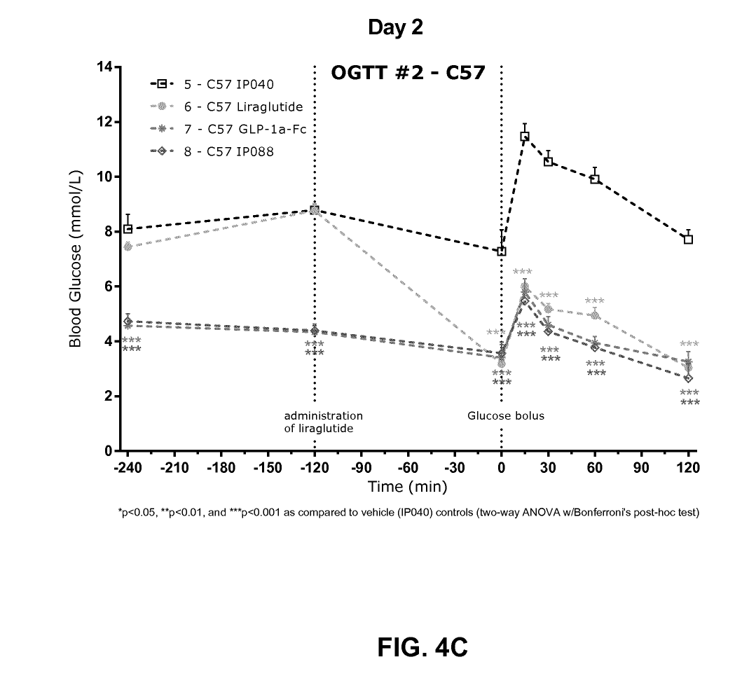

A schematic summary of the study is shown in FIG. 4A. Oral glucose tolerance tests (OGTT) were performed on days 0 and 2, both after a 4-hour fast. At t=0 the mice were administered an oral glucose load of 2 g/kg glucose (Glucose 250 m/ml, Fresenius Kabi, Sweden). Glucose was given as gavage via a gastrically placed tube connected to a syringe ensuring accurate dosing. Blood glucose was measured from tail vein blood before glucose (at t=−240, −120 and 0 min, baseline) and after glucose administration at t=15, 30, 60 and 120 min. Blood samples (tail vein) were collected into 10 μm heparinized glass capillary tubes and immediately suspended in buffer (0.5 ml of glucose/lactate system solution (EKF-diagnostics, Germany) and analyzed for glucose on the test day using a BIOSEN c-Line glucose meter (EKF-diagnostics, Germany) according to manufacturer's instructions.

The results are shown in FIGS. 4B (day 0 OGTT) and 4C (day 2 OGTT). While this study could not distinguish the relative contribution of GLP-1 and GIP agonism, IP088 showed excellent glucose lowering effects, and was superior to GLP1a-Fc at glycemic control.

The disclosure is not to be limited in scope by the specific aspects described, which are intended as single illustrations of individual aspects of the disclosure, and any compositions or methods that are functionally equivalent are within the scope of this disclosure. Indeed, various modifications of the disclosure in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

What is claimed is:

1. An isolated polypeptide comprising the amino acid sequence:
(SEQ ID NO: 461)
Y S X₃ G T F T S D X₁₀ S X₁₂ X₁₃ X₁₄ X₁₅ X₁₆ X₁₇
X₁₈ X₁₉ X₂₀ X₂₁ F X₂₃ X₂₄ W L X₂₇ X₂₈ X₂₉ X₃₀;
wherein X₃ is E or Q; X₁₀ is Y, V, or L; X₁₂ is K, S, or I; X₁₃ is L, Y, or A; X₁₄ is L or M; X₁₅ is E or D; X₁₆ is E, G, or K; X₁₇ is E, Q, or I; X₁₈ is A or H; X₁₉ is V, A, or Q; X₂₀ is R, K, or Q; X₂₁ is L, E, or D; X₂₃ is I or V; X₂₄ is E, A, or N; X₂₇ is L, K, or V; X₂₈ is A, K, or N; X₂₉ is G or Q; and X₃₀ is no amino acid, G, N, P, K, or T.

2. The polypeptide of claim 1, wherein X₁₃ is L.

3. The polypeptide of claim 2, wherein X₃ is E, X₁₀ is Y, X₁₂ is K, X₁₄ is L, X₁₅ is E, X₁₆ is E, X₁₇ is E, X₁₈ is A, X₁₉ is V, X₂₀ is R, X₂₁ is L, X₂₃ is I; X₂₄ is E; and X₂₉ is G.

4. The polypeptide of any one of claims 1 to 3, wherein X₂₇ is L and X₂₈ is A; X₂₇ is K and X₂₈ is N; or X₂₇ is V and X₂₈ is K.

5. The polypeptide of claim 1, comprising an amino acid sequence selected from the group consisting of SEQ ID NOs:2-32.

6. An isolated polypeptide comprising the amino acid sequence:
(SEQ ID NO: 190)
X₁ X₂ E G T F T S D X₃ S X₄ X₅ X₆ X₇ X₈ X₉ A I D
E F X₁₀ X₁₁ X₁₂ L L X₁₃ X₁₄ X₁₅;
wherein X₁ is H or Y; X₂ is G or S; X₃ is V or Y; X₄ is S, K, or I; X₅ is Y, L, or A; X₆ is M or L; X₇ is E or D; X₈ is R or E; X₉ is Q or E; X₁₀ is I or V; X₁₁ is A or N; X₁₂ is W or D; X₁₃ is A, K, or G; X₁₄ is G or Q; and X₁₅ is no amino acid, G, or K.

7. The polypeptide of claim 6, wherein X₁ is Y and X₂ is S.

8. The polypeptide of any one of claim 6 or claim 7, wherein X₁₀ is I, X₁₁ is A, and X₁₂ is W.

9. The polypeptide of any one of claims 6 to 8, wherein X₁₄ is G and X₁₅ is no amino acid or G.

10. The polypeptide of any one of claims 6 to 9, wherein X₇ is E.

11. The polypeptide of any one of claims 6 to 10, wherein X₈ is R and X₉ is Q.

12. The polypeptide of any one of claims 6 to 11, wherein X₁₃ is K.

13. The polypeptide of any one of claims 6 to 12, wherein X₃ is V.

14. The polypeptide of any one of claims 6 to 14, wherein X₄ is S, X₅ is Y, and X₆ is M.

15. The polypeptide of claim 6, comprising an amino acid sequence selected from the group consisting of SEQ ID NOs:33-43.

16. An isolated polypeptide comprising the amino acid sequence of IP171, IP176, IP206, IP214, IP215, IP175. IP208, IP204, IP199, IP173, IP150, IP174, IP205, IP127, or IP200.

17. An isolated polypeptide comprising the amino acid sequence:
(SEQ ID NO: 462)
X₁ X₂ X₃ G X₅ X₆ X₇ S X₉ X₁₀ X₁₁ X₁₂ X₁₃ X₁₄ X₁₅
X₁₆ X₁₇ X₁₈ X₁₉ X₂₀ X₂₁ F X₂₃ X₂₄ X₂₅
L X₂₇ X₂₈ X₂₉ X₃₀;
wherein X₁ is Y or H; X₂ is S or G; X₃ is E or Q; X₅ is T or M; X₆ if F or H; X₇ is T or I; X₉ is D or L; X₁₀ is Y, V, F, or L; X₁₁ is S, R, or M; X₁₂ is K, S, M, or I; X₁₃ is L, Y, Q, H, I or A; X₁₄ is L, K, or M; X₁₅ is E or D; X₁₆ is E, G, or K; X₁₇ is E, Q, or I; X₁₈ is A, C, or H; X₁₉ is V, A, E, I, N, S, T, or Q; X₂₀ is R, K, or Q; X₂₁ is L, E, C, or D; X₂₃ is I or V; X₂₄ is E, C, A, or N; X₂₅ is W, C, F, H, Y, or K; X₂₇ is L, K, or V; X₂₈ is A, K, C, or N; X₂₉ is G or Q; and X₃₀ is no amino acid, G, N, P, K, or T.

18. The polypeptide of claim 17, comprising an amino acid sequence selected from the group consisting of SEQ ID NOs: 2-32, and 191-256.

19. An isolated polypeptide comprising the amino acid sequence:
(SEQ ID NO: 463)
X₁ X₂ E G X₅ X₆ X₇ S X₉ X₁₀ X₁₁ X₁₂ X₁₃ X₁₄ X₁₅
X₁₆ X₁₇ A I X₂₀ X₂₁ F X₂₃ X₂₄ X₂₅ L X₂₇ X₂₈ X₂₉
X₃₀;
wherein X₁ is H or Y; X₂ is G or S; X₅ is T or M; X₆ is F or H; X₇ is T or I; X₉ is D or L; X₁₀ is V, F, L, or Y; X₁₁ is S, A, or R; X₁₂ is S, K, A, M, Q or I; X₁₃ is Y, L, H, or A; X₁₄ is M, A, K, I, or L; X₁₅ is E or D; X₁₆ is R, K, or E; X₁₇ is Q or E; X₂₀ is D or E; X₂₁ is E or A; X₂₃ is I, A, or V; X₂₄ is A or N; X₂₅ is W, E, L, F, I, S, N, G, M, H, K or D; X₂₇ is L or A; X₂₈ is A, K, or G; X₂₉ is G, A, or Q; and X₃₀ is no amino acid, G, A, E, or K.

20. The polypeptide of claim 19, comprising an amino acid sequence selected from the group consisting of SEQ ID NOs: 33-43 and 257-294.

21. An isolated polypeptide comprising the amino acid sequence:
(SEQ ID NO: 1)
Y S X₁ G T F T S D X₂ S X₃ X₄ X₅ E X₆ X₇ X₈ X₉ X₁₀
X₁₁ X₁₂ F X₁₃ X₁₄ W L X₁₅ X₁₆ X₁₇ X₁₈;
wherein X₁ is E or Q; X₂ is Y, V, or L; X₃ is K, S, or I; X₄ is L, Y, or A; X₅ is L or M; X₆ is E or D; X₇ is E, G, or K; X₈ is E, Q, or I; X₉ is A or H; X₁₀ is V, A, or Q; X₁₁ is R, K, or Q; X₁₂ is L, E, or D; X₁₃ is I or V; X₁₄ is E, A, or N; X₁₅ is L, K, or V; X₁₆ is A, K, or N; X₁₇ is G or Q; and X₁₈ is no amino acid, G, N, P, K, or T.

22. The polypeptide of claim 21, wherein X₄ is L.

23. The polypeptide of claim 22, wherein X₁ is E; X₂ is Y; X₃ is K; X₅ is L; X₆ is E; X₇ is E; X₈ is E; X₉ is A; X₁₀ is V; X₁₁ is R, X₁₂ is L; X₁₃ is I; X₁₄ is E; and X₁₇ is G.

24. The polypeptide of any one of claims 21 to 23, wherein X₁₅ is L and X₁₆ is A; X₁₅ is K and X₁₆ is N; or X₁₅ is V and X₁₆ is K.

25. The polypeptide of claim 21, comprising an amino acid sequence selected from the group consisting of SEQ ID NOs:2-32.

26. The polypeptide of any one of claims 1 to 25, further comprising a heterologous polypeptide fused thereto.

27. The polypeptide of claim 26, wherein the heterologous polypeptide comprises a linker, a hinge, an Fc domain, or a combination thereof.

28. The polypeptide of claim 27, wherein the linker comprises (GGGGS)n, wherein n 1, 2, 3, or 4.

29. The polypeptide of claim 27 or claim 28, wherein the linker comprises the amino acid sequence: G GGGGS GGGGS GGGGS GGGGS A (SEQ ID NO:66), A PPGGS GGGGS GGGGS GGGGS A (SEQ ID NO:67), GT GGGGS GGGGS GGGGS GGGGS A (SEQ ID NO:68), G GGGGS GGGGS GGGGS GGGGS A (SEQ ID NO:69), G GGGGS A (SEQ ID NO:70), G GGGGS GGGGS A (SEQ ID NO:71), G GGGGS GGGGS GGGGS A (SEQ ID NO:72), G KGGGS GGGGS GGGGS GGGGS A (SEQ ID NO:73), G GGGGS GGGGS GGGGS GGGGSA (SEQ ID NO:179), G GGGG GGGG GGGG GGGG A (SEQ ID NO:180) or any combination thereof.

30. The polypeptide of claim 27, wherein the linker comprises GG SGSTA SSGSG SATGG GGAA (SEQ ID NO:74), any variant thereof, or any fragment thereof.

31. The polypeptide of claim 30, wherein the linker comprises AAAGG SGSTA SSGSG SATGG GGAA (SEQ ID NO:75), APPGG SGSTA SSGSG SATGG GGAA (SEQ ID NO:76), or any combination thereof.

32. The polypeptide of claim 27, wherein the hinge comprises an amino acid sequence of an IgG1 hinge, an IgG4 hinge, a fragment thereof, a variant thereof, or any combination thereof.

33. The polypeptide of claim 32, wherein the hinge comprises ESKYGPPCPPCPAPEAA (SEQ ID NO:77), THTCPPCPAPEF (SEQ ID NO:78), THTCPPC (SEQ ID NO:79), CPPCPAPEF (SEQ ID NO:80), TYTCPPCPAPEF (SEQ ID NO:81), TSTCPPCPAPEF (SEQ ID NO:82), PPCPPCPAPEF (SEQ ID NO:83), ESKYGPPCPPCPAPEF (SEQ ID NO:84), APEF (SEQ ID NO:85), ESKYGPPCPPC (SEQ ID NO:86), THTCPPCPAPELL (SEQ ID NO:87), any variant thereof, any fragment thereof, or any combination thereof.

34. The polypeptide of claim 27, wherein the Fc region comprises an IgG1 Fc region, an IgG1-TM Fc region, an IgG1-FQQ Fc region, an IgG4 Fc region, an IgG1-YTE Fc region, any fragment thereof, any variant thereof, or any combination thereof.

35. The polypeptide of claim any one of claims 26 to 34, comprising an amino acid sequence selected from the group consisting of SEQ ID NOs:88-175 and 326-460.

36. The polypeptide of any one of claims 1 to 35, further comprising a heterologous moiety attached thereto.

37. The polypeptide of claim 36, wherein the heterologous moiety is a polypeptide, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, or a combination of two or more of the recited moieties.

38. The polypeptide of any one of claims 1 to 37, wherein the peptide binds to a GIP receptor, binds to a GLP-1 receptor, or binds to both a GIP and a GLP-1 receptor.

39. The polypeptide of claim 38, which binds to a GIP receptor.

40. The polypeptide of claim 39, wherein the GIP receptor is a mouse GIP receptor, a rat GIP receptor, or a human GIP receptor.

41. The polypeptide any one of claims 38 to 40, which binds to a human GIP receptor with an EC50 in the cAMP assay 1 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

42. The polypeptide of any one of claims 38 to 41, which binds to a human glucagon receptor with at least 1000-fold lower affinity than its binding affinity for a human GIP receptor, as measured in the cAMP assay.

43. The polypeptide of any one of claims 38 to 42, which binds to a GLP-1 receptor.

44. The polypeptide of claim 43, wherein the GLP-1 receptor is a mouse GLP-1 receptor, a rat GLP-1 receptor, or a human GLP-1 receptor.

45. The polypeptide of claim 43 or claim 44, which binds to a human GLP-1 receptor with an EC50 in the cAMP assay 1 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

46. The polypeptide of any one of claims 43 to 45, which binds to a human glucagon receptor with at least 1000-fold lower affinity than its binding affinity for a human GLP-1 receptor, as measured in the cAMP assay.

47. The polypeptide of any one of claims 38 to 46, which is an agonist of GLP-1 activity, an agonist of GIP activity, or an agonist of both GLP-1 and GIP activity.

48. An isolated polynucleotide encoding the polypeptide of any one of claims 1 to 47.

49. A vector comprising the polynucleotide of claim 48.

50. A host cell comprising the polynucleotide of claim 48 or the vector of claim 49.

51. A method of making the polypeptide of any one of claims 1 to 47, comprising culturing the host cell of claim 50 under conditions allowing expression of the polypeptide, and recovering the polypeptide.

52. A pharmaceutical composition comprising the polypeptide of any one of claims 1 to 47, and a carrier.

53. A kit comprising the composition of claim 52.

54. A method of treating or preventing a disease or condition caused or characterized by hypoglycemia or impaired insulin release, comprising administering to a subject in need of treatment an effective amount of the polypeptide of any one of claims 1 to 47 or the composition of claim 52.

55. The method of claim 54, wherein the disease or condition is diabetes.

56. The method of claim 55, wherein the disease or condition is type-2 diabetes.

57. The method of any one of claims 54 to 56, wherein the administration further improves glycemic control, provides body weight control, improves β-cell function and mass, reduces the rate of gastric acid secretion and gastric emptying, or any combination thereof.

58. The method of any one of claims 54 to 57, wherein the peptide is administered orally or by injection.

59. The method of claim 58, wherein the injection is administered subcutaneously or intravenously.

60. The method of claim 58 or claim 59, wherein the polypeptide is administered once per day.

61. The method of any one of claims 54 to 60, further comprising administering one or more additional therapies.

62. The method of claim 61, wherein the additional therapy comprises blood sugar monitoring, diet modifications, exercise, insulin, a thiazolidinedione, a sulfonylurea, an incretin, metformin, a glyburide, a dipeptidyl peptidase 4 inhibitor, a bile acid sequestrant, or any combination thereof.

63. The method of any one of claims 54 to 62, wherein the subject is human.
